(19) United States
(12) Patent Application Publication
(10) Pub. No.: US 2025/0313553 A1
(43) Pub. Date: Oct. 9, 2025

(54) ANNULATED 2-AMINO-3-CYANO THIOPHENES AND DERIVATIVES FOR THE TREATMENT OF CANCER

(71) Applicants: Boehringer Ingelheim Int'l GmbH Corp., Ingelheim (DE); Vanderbilt University, Nashville, TN (US)

(72) Inventors: Joachim BROEKER, Moedling (AT); Jason ABBOTT, Libertyville, TN (US); Jianwen CUI, Franklin, TN (US); Stephen W. FESIK, Nashville, TN (US); Andreas GOLLNER, Vienna (AT); Tim HODGES, Chicago, IL (US); Andrew LITTLE, Sudbury, MA (US); Andreas MANTOULIDIS, Vienna (AT); Jason PHAN, Nashville, TN (US); Dhruba SARKAR, Bangalore (IN); Christian Alan Paul SMETHURST, Ingelheim (DE); Qi SUN, Libertyville, IL (US); Alex WATERSON, Murfreesboro, TN (US)

(21) Appl. No.: 18/715,200

(22) PCT Filed: Nov. 30, 2022
(86) PCT No.: PCT/EP2022/083906

§ 371 Date: May 31, 2024

Related U.S. Application Data
(60) Provisional application No. 63/284,751, filed on Dec. 1, 2021.
(60) Provisional application No. 63/284,788, filed on Dec. 1, 2021.

(51) Int. Cl. C07D 413/14; A61K 31/4439; A61K 31/496; A61K 31/506; A61K 31/551; A61P 35/00; C07D 417/14

(52) U.S. Cl. CPC C07D 413/14; A61K 31/4439; A61K 31/496; A61K 31/506; A61K 31/551; A61P 35/00; C07D 417/14

(57) ABSTRACT

The present invention encompasses compounds of formula (V), wherein R^{1a}, R^{1b}, R^{2a}, R^{2b}, Z, R⁴, R⁵, R¹⁴, A, p, U, V and W have the meanings given in the claims and specification, their use as inhibitors of KRAS, pharmaceutical compositions and preparations containing such compounds and their use as medicaments/medical uses, especially as agents for treatment and/or prevention of oncological diseases.

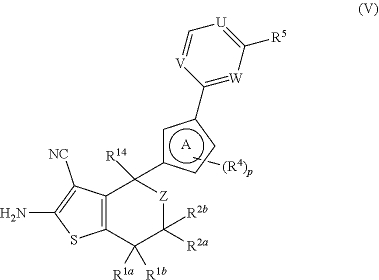

### CROSS-REFERENCE TO RELATED APPLICATIONS

This application is a U.S. Nat'l Phase of Int'l Appl. No. PCT/EP2022/083906 filed Nov. 30, 2022, which claims the benefit of priority to U.S. Provisional Appl. No. 63/284,751 filed Dec. 1, 2021, and to U.S. Provisional Appl. No. 63/284,788 filed Dec. 1, 2021, all of which are hereby incorporated herein by reference in their entireties.

### FIELD OF THE INVENTION

The present invention relates to annulated 2-amino-3-cyano thiophenes and derivatives of formula (V):

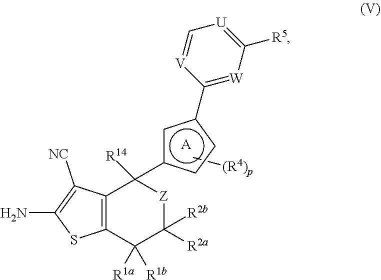

wherein R^{1a}, R^{1b}, R^{2a}, R^{2b}, Z, R⁴, R⁵, R¹⁴, A, p, U, V and W have the meanings given in the claims and specification, their use as inhibitors of KRAS, pharmaceutical compositions and preparations containing such compounds and their use as medicaments/medical uses, especially as agents for treatment and/or prevention of oncological diseases, e.g. cancer.

### BACKGROUND OF THE INVENTION

V-Ki-ras2 Kirsten rat sarcoma viral oncogene homolog (KRAS) is a small GTPase of the Ras family of proteins that exists in cells in either GTP-bound or GDP-bound states (McCormick et al., J. Mol. Med. (Berl)., 2016, 94(3):253-8; Nimnual et al., Sci. STKE., 2002, 2002(145):pe36). Binding of GTPase activating proteins (GAPs) such as NF1 increases the GTPase activity of Ras family proteins. The binding of guanine nucleotide exchange factors (GEFs) such as SOS1 (Son of Sevenless 1) promotes release of GDP from Ras family proteins, enabling GTP binding (Chardin et al., Science, 1993, 260(5112):1338-43). When in the GTP-bound state, Ras family proteins are active and engage effector proteins including C-RAF and phosphoinositide 3-kinase (PI3K) to promote the RAF/mitogen or extracellular signal-regulated kinases (MEK/ERK) pathway, PI3K/AKT/mammalian target of rapamycin (mTOR) pathway and RaIGDS (Ral guanine nucleotide dissociation stimulator) pathway (McCormick et al., J. Mol. Med. (Berl)., 2016, 94(3):253-8; Rodriguez-Viciana et al., Cancer Cell. 2005, 7(3):205-6). These pathways affect diverse cellular processes such as proliferation, survival, metabolism, motility, angiogenesis, immunity and growth (Young et al., Adv. Cancer Res., 2009, 102:1-17; Rodriguez-Viciana et al., Cancer Cell. 2005, 7(3):205-6).

Cancer-associated mutations in Ras family proteins suppress their intrinsic and GAP-induced GTPase activity leading to an increased population of GTP-bound/active mutant Ras family proteins (McCormick et al., Expert Opin. Ther. Targets., 2015, 19(4):451-4; Hunter et al., Mol. Cancer Res., 2015, 13(9):1325-35). This in turn leads to persistent activation of effector pathways (e.g. RAF/MEK/ERK, PI3K/AKT/mTOR, RaIGDS pathways) downstream of mutant Ras family proteins. KRAS mutations (e.g. amino acids G12, G13, Q61, A146) are found in a variety of human cancers including lung cancer, colorectal cancer and pancreatic cancer (Cox et al., Nat. Rev. Drug Discov., 2014, 13(11):828-51). Alterations (e.g. mutation, overexpression, gene amplification) in Ras family proteins/Ras genes have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Ortiz-Cuaran et al., Clin. Cancer Res., 2016, 22(19):4837-47; Eberlein et al., Cancer Res., 2015, 7 5(12):2489-500).

In a subset of tumor indications such as gastric cancer, gastroesophageal junction cancer and esophageal cancer prominent amplification of the wildtype (WT) KRAS proto-oncogene acts as a driver alteration and renders tumor models bearing this genotype addicted to KRAS in vitro and in vivo (Wong et al. Nat Med., 2018, 24(7):968-977). In contrast, non-amplified KRAS WT cell lines are KRAS independent, unless they carry secondary alterations in genes indirectly causing activation of KRAS (Meyers et al., Nat Genet., 2017, 49:1779-1784). Based on these data, a therapeutic window is expected for a KRAS targeting agent with a KRAS WT targeting activity.

Genetic alterations affecting e.g. codon 12 of KRAS substitute the glycine residue naturally occurring at this position for different amino acids such as aspartic acid (the G12D mutation or KRAS G12D), cysteine (the G12C mutation or KRAS G12C), valine (the G12V mutation or KRAS G12V) among others. Similarly, mutations within codons 13, 61 and 146 of KRAS are commonly found in the KRAS gene. Altogether KRAS mutations are detectable in 35% of lung, 45% of colorectal, and up to 90% of pancreatic cancers (Herdeis et al., Curr Opin Struct Biol., 2021, 71:136-147).

In summary, binders/inhibitors of wildtype or mutated KRAS (e.g., G12D, G12V and G12C) are expected to deliver anti-cancer efficacy.

Thus, there is the need to develop new compounds efficacious in the treatment of cancers mediated by KRAS, especially KRAS mutated in position 12 or 13 and/or in wild-type amplified KRAS mediated cancer, which also possess desirable pharmacological properties, including but not limited to: metabolic stability, plasma protein binding, solubility and permeability.

### DETAILED DESCRIPTION OF THE INVENTION

It has now been found that, surprisingly, compounds of formula (V)

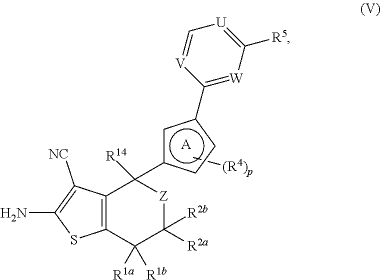

wherein R^{1a}, R^{1b}, R^{2a}, R^{2b}, Z, R⁴, R⁵, R¹⁴, A, p, U, V and W have the meanings given hereinafter act as inhibitors of KRAS and are involved in controlling cell proliferation. Thus, the compounds according to the invention may be used for example for the treatment of diseases characterized by excessive or abnormal cell proliferation.

Surprisingly, the compounds described herein have been found to possess anti-tumour activity, being useful in inhibiting the uncontrolled cellular proliferation which arises from malignant diseases. It is believed that this anti-tumor activity is, inter alia, derived from inhibition of KRAS mutated in position 12 or 13, preferably G12D, G12V or G13D mutant KRAS, or inhibition of WT KRAS, especially KRAS WT amplified. Advantageously, the compounds can be selective for certain KRAS mutants, preferably KRAS G12D, or can be effective against a panel of KRAS mutants including KRAS wildtype amplified.

In addition, the compounds of the invention advantageously possess desirable pharmacological properties, including but not limited to: metabolic stability, plasma protein binding, solubility and permeability.

Thus, in a first aspect, the present invention relates to a compound of the formula (V)

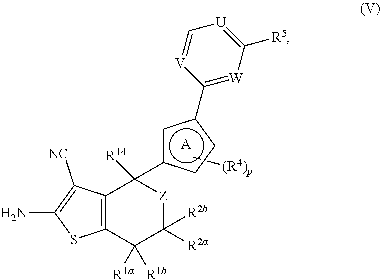

wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached form a cyclopropane ring;
Z is —(CR^{6a}R^{6b})ₙ—;
each R^{6a} and R^{6b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{6a} and R^{6b} together with the carbon atom they are attached to form a cyclopropane ring; n is selected from the group consisting of 0, 1 and 2;
R¹⁴ is selected from the group consisting of hydrogen, C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
W is nitrogen (—N═) or —CH═;
V is nitrogen (—N═) or —CH═;
U is nitrogen (—N═) or —C(R¹¹)═;
R¹¹ is selected from hydrogen, halogen and C₁₋₄alkoxy;
ring A is a 5 membered heteroarylene;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl; p is selected from the group consisting of 0, 1, 2 and 3;
R⁵ is halogen or a 3-11 membered heterocyclyl optionally substituted with one or more identical or different C₁₋₆alkyl, C₁₋₆alkoxy, —C(O)—O—C₁₋₆alkyl or a 5-6 membered heterocyclyl, wherein the C₁₋₆alkyl is optionally substituted with cyclopropyl;
or R⁵ is —O—C₁₋₆alkyl substituted with a 3-11 membered heterocyclyl, wherein the 3-11 membered heterocyclyl is optionally substituted with one or more, identical or different R¹²
   each R¹² is selected from the group consisting of C₁₋₆alkyl, C₁₋₆alkoxy, —C(O)—O—C₁₋₆alkyl halogen and 3-11 membered heterocyclyl;
or a salt thereof.

In another aspect, the present invention relates to a compound of formula (V*)

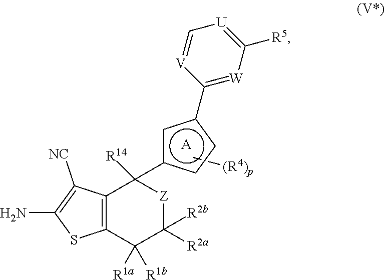

wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached form a cyclopropane ring;
Z is —(CR^{6a}R^{6b})ₙ—;
each R^{6a} and R^{6b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{6a} or R^{6b} together with the carbon atom they are attached to form a cyclopropane ring;
n is selected from the group consisting 0, 1 and 2;
R¹⁴ is selected from the group consisting of hydrogen, C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
W is nitrogen (—N═) or —CH═;
V is nitrogen (—N═) or —CH═;
U is nitrogen (—N═) or —C(R¹¹)═;
   each R¹¹ is selected from hydrogen, halogen and C₁₋₄alkoxy;
ring A is a 5 membered heteroarylene;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting 0, 1, 2 and 3;
R⁵ is halogen or a 3-11 membered heterocyclyl optionally substituted with one or more identical or different C₁₋₆alkyl, C₁₋₆alkoxy or a 5-6 membered heterocyclyl, wherein the C₁₋₆alkyl is optionally substituted with cyclopropyl;
or R⁵ is —O—C₁₋₆alkyl substituted with a 3-11 membered heterocyclyl, wherein the 3-11 membered heterocyclyl is optionally substituted with one or more, identical or different R¹²,
each R¹² is selected from the group consisting of C₁₋₆alkyl, C₁₋₆alkoxy, halogen and 3-11 membered heterocyclyl;
or a salt thereof.

It is to be understood that the following embodiments and aspects can be applied to formula (V) and to formula (V*), unless stated otherwise.

In another aspect, the present invention relates to a compound of formula (V′), or a salt thereof,

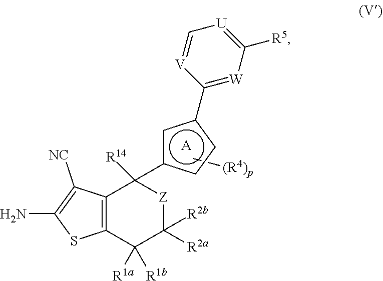

wherein R^{1a}, R^{1b}, R^{2a}, R^{2b}, Z, R⁴, R⁵, A, p, U, V and W have the meanings given herein.

In another aspect, the present invention relates to a compound of formula (V″), or a salt thereof,

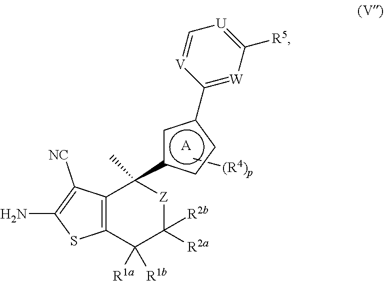

wherein R^{1a}, R^{1b}, R^{2a}, R^{2b}, Z, R⁴, R⁵, A, p, U, V and W have the meanings given herein.

In another aspect, the invention relates to the compound of the invention, or a salt thereof, wherein R^{1a} and R^{1b} are both independently selected from the group consisting of hydrogen and C₁₋₄alkyl.

In another aspect, the invention relates to the compound of the invention, or a salt thereof, wherein R^{2a} and R^{2b} are both independently selected from the group consisting of hydrogen and halogen.

In another aspect, the invention relates to the compound of the invention, or a salt thereof, wherein R^{1a} and R^{1b} are both independently selected from the group consisting of hydrogen and methyl.

In another aspect, the invention relates to the compound of the invention, or a salt thereof, wherein R^{2a} and R^{2b} are both independently selected from the group consisting of hydrogen and fluorine.

In another aspect, the invention relates to the compound of the invention, or a salt thereof, wherein R^{1a}, R^{1b}, R^{2a} and R^{2b} are hydrogen.

In another aspect, the invention relates to the compound of the invention, or a salt thereof, wherein Z is —(CR^{6a}R^{6b})ₙ— and n is selected from the group consisting 0.

In another aspect, the invention relates to the compound of the invention, or a salt thereof, wherein Z is —(CR^{6a}R^{6b})ₙ—; n is 1; and each R^{6a} and R^{6b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl.

In another aspect, the invention relates to the compound of the invention, or a salt thereof, wherein Z is —CH₂—.

In another aspect, the invention relates to the compound of the invention, or a salt thereof, wherein R^{1a}, R^{1b}, R^{2a} and R^{2b} are hydrogen and Z is —CH₂—.

In another aspect, the invention relates to the compound of the invention, or a salt thereof, wherein Z is —(CR^{6a}R^{6b})ₙ—; n is 2; each R^{6a} and R^{6b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl.

In another aspect, the present invention relates to a compound of formula (Va), or a salt thereof,

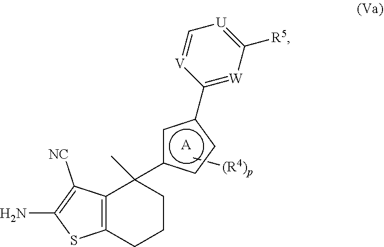

wherein A, V, U, W, p, R⁴ and R⁵ are defined herein.

In another aspect the present invention relates to a compound of the formula (Vb) or a salt thereof,

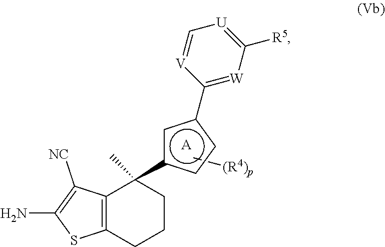

wherein A, V, U, W, p, R⁴ and R⁵ are defined herein.

In another aspect, the invention relates to the compound of the invention, or a salt thereof, wherein p is 0.

In another aspect, the invention relates to the compound of the invention, or a salt thereof, wherein, ring A is a ring selected from the group consisting of pyrrole, furan, thiophene, imidazole, pyrazole, oxazole, isoxazole, thiazole, isothiazole and triazole.

In another aspect, the invention relates to the compound of the invention, or a salt thereof, wherein ring A is selected from the group consisting of

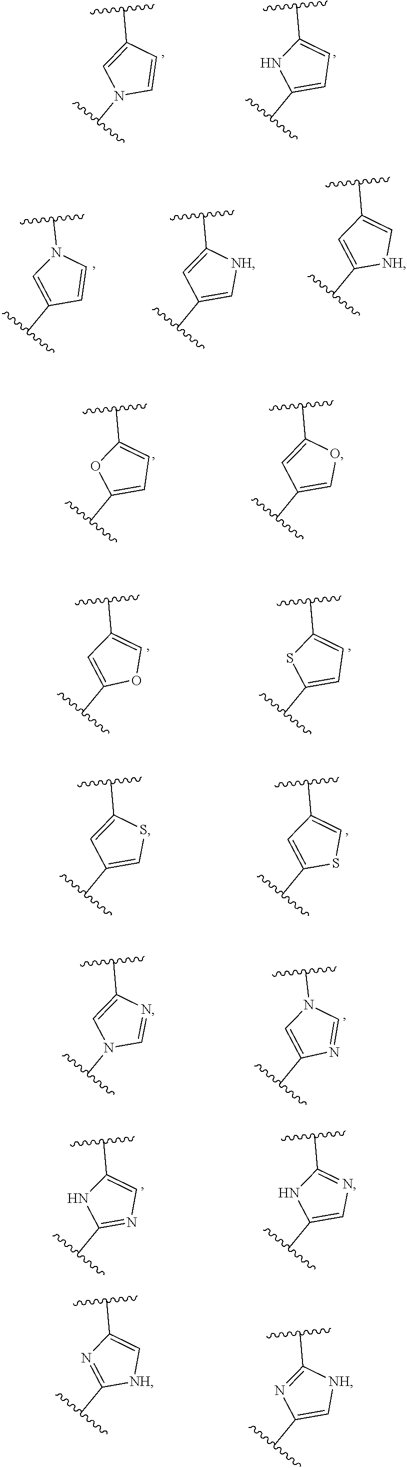

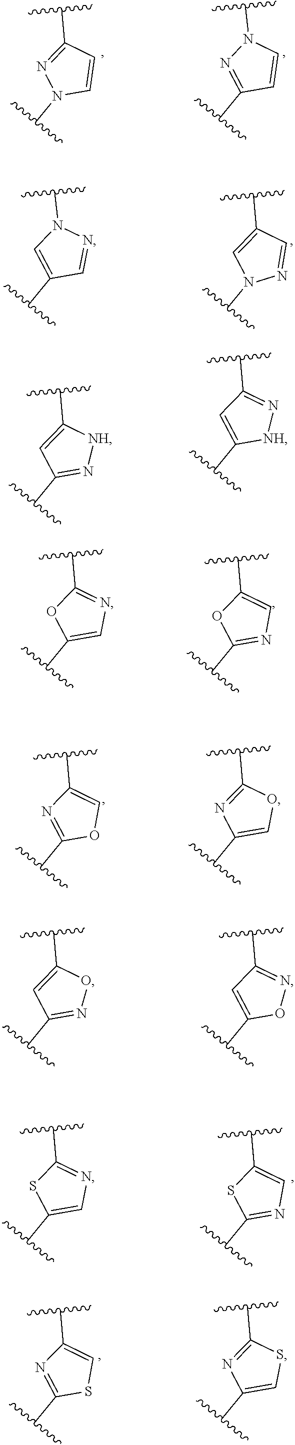

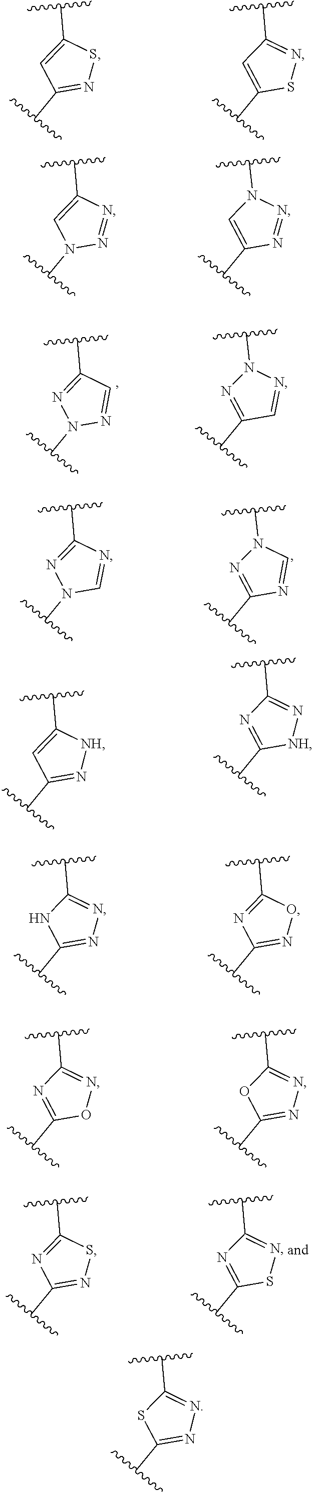

In another aspect, the invention relates to the compound of the invention, or a salt thereof, wherein ring A is selected from

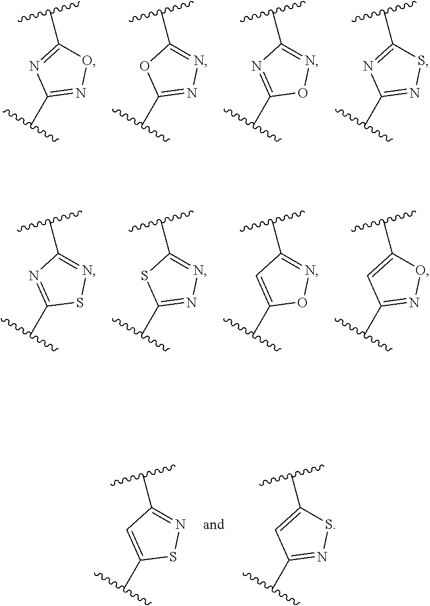

In another aspect, the invention relates to the compound of the invention, or a salt thereof, wherein ring A is selected from

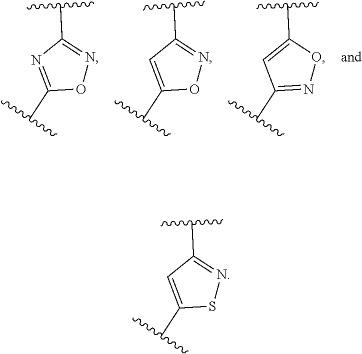 [SMILES: *c1noc(*)n1 ; *c1cc(*)on1 ; *c1cc(*)on1 ; *c1cc(*)sn1]

In another aspect, the invention relates to the compound of the invention, or a salt thereof, wherein ring A is

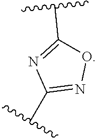 [SMILES: *c1noc(*)n1]

In another aspect, the invention relates to a compound of formula (Vc) or a salt thereof,

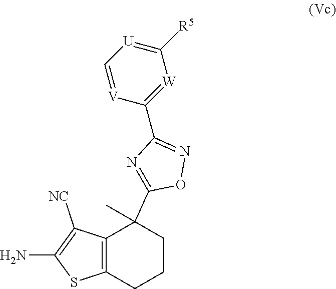

wherein V, U, W and R⁵ are defined herein.

In another aspect, the invention relates to a compound of formula (Vd) or a salt thereof,

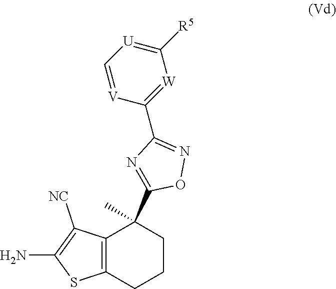

wherein V, U, W, and R⁵ are defined herein.

In another aspect, the invention relates to a compound of formula (Ve) or a salt thereof,

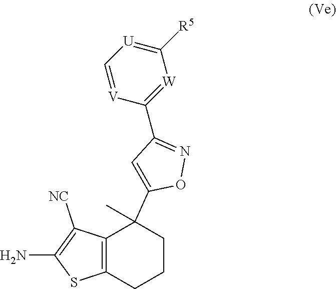

wherein V, U, W and R⁵ are defined herein.

In another aspect, the invention relates to a compound of formula (Vf) or a salt thereof,

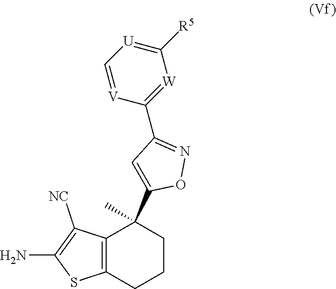

wherein V, U, W, and R⁵ are defined herein.

In another aspect, the invention relates to a compound of formula (Vg) or a salt thereof,

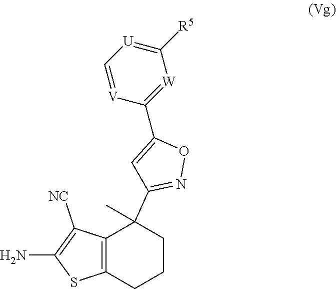

wherein V, U, W and R⁵ are defined herein.

In another aspect, the invention relates to a compound of formula (Vh) or a salt thereof,

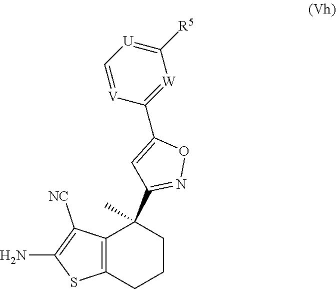

wherein V, U, W, and R⁵ are defined herein.

In another aspect, the invention relates to a compound of formula (Vi) or a salt thereof,

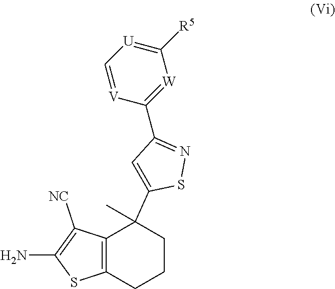

wherein V, U, W and R⁵ are defined herein.

In another aspect, the invention relates to a compound of formula (Vj) or a salt thereof,

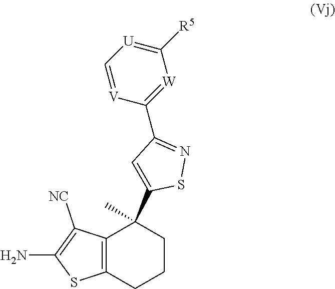

wherein V, U, W, and R⁵ are defined herein.

In another aspect, the invention relates to the compound of the invention, or a salt thereof, wherein at least one of W, V and U is nitrogen.

In another aspect, the invention relates to the compound of the invention, or a salt thereof, wherein W is nitrogen (—N═); V is nitrogen (—N═); U is ═C(R¹¹)—; R¹¹ is selected from hydrogen, halogen and C₁₋₄alkoxy.

In another aspect, the invention relates to the compound of the invention, or a salt thereof, wherein W is —CH═; V is nitrogen (—N═); U is ═C(R¹¹)—; each R¹¹ is selected from hydrogen, halogen and C₁₋₄alkoxy.

In another aspect, the invention relates to the compound of the invention, or a salt thereof, wherein V is —CH═; W is nitrogen (—N═); U is ═C(R¹¹)—; each R¹¹ is selected from hydrogen, halogen and C₁₋₄alkoxy.

In another aspect, the invention relates to the compound of the invention, or a salt thereof, wherein R¹¹ is selected from hydrogen, —F, —Cl and —O—CH₃.

In another aspect, the invention relates to the compound of invention, or a salt thereof, wherein V is nitrogen (—N═); W is —CH═; U is nitrogen (—N═).

In another aspect, the invention relates to the compound of the invention, or a salt thereof, wherein W is nitrogen (—N═); V is —CH═; U is nitrogen (—N═).

In another aspect, the invention relates to the compound of the invention, or a salt thereof, wherein W is —CH═; V is —CH═; U is nitrogen (—N═).

In another aspect, the invention relates to the compound of invention, or a salt thereof, wherein W is nitrogen (—N═); V is nitrogen (—N═); U is nitrogen (—N═).

In another aspect, the invention relates to the compound of the invention, wherein R⁵ is halogen or a 3-11 membered heterocyclyl optionally substituted with one or more identical or different C₁₋₆alkyl, C₁₋₆alkoxy or a 5-6 membered heterocyclyl, wherein the C₁₋₆alkyl is optionally substituted with cyclopropyl;
or R⁵ is —O—C₁₋₆alkyl substituted with a 3-11 membered heterocyclyl, wherein the 3-11 membered heterocyclyl is optionally substituted with one or more, identical or different R¹²,
   each R¹² is selected from the group consisting of C₁₋₆alkyl, C₁₋₆alkoxy, halogen and 3-11 membered heterocyclyl.

In another aspect, the invention relates to the compound of the invention, or a salt thereof, wherein R⁵ is chlorine.

In another aspect, the invention relates to the compound of the invention, or a salt thereof, wherein R⁵ is fluorine.

In another aspect, the invention relates to the compound of the invention, or a salt thereof, wherein R⁵ is halogen or a 6-11 membered heterocyclyl optionally substituted with one or more identical or different C₁₋₆alkyl, C₁₋₆alkoxy, —C(O)—O—C₁₋₆alkyl or a 5-6 membered heterocyclyl, wherein the C₁₋₆alkyl is optionally substituted with cyclopropyl.

In another aspect, the invention relates to the compound of the invention, or a salt thereof, wherein R⁵ is halogen or a 6-11 membered heterocyclyl optionally substituted with one or more identical or different C₁₋₆alkyl, C₁₋₆alkoxy or a 5-6 membered heterocyclyl, wherein the C₁₋₆alkyl is optionally substituted with cyclopropyl.

In another aspect, the invention relates to the compound of the invention, or a salt thereof, wherein R⁵ is selected from the group consisting of 7 membered heterocyclyl, optionally substituted with one or more independently selected C₁₋₄alkyl.

In another aspect, the invention relates to the compound of the invention, or a salt thereof, wherein R⁵ is —O—C₁₋₆alkyl substituted with a 5-9 membered heterocyclyl, wherein the 5-9 membered heterocyclyl is optionally substituted with one or more, identical or different R¹², each R¹² is selected from the group consisting of C₁₋₆alkyl, C₁₋₆alkoxy, —C(O)—O—C₁₋₆alkyl halogen and 5-6 membered heterocyclyl.

In another aspect, the invention relates to the compound of the invention, or a salt thereof, wherein R⁵ is —O—C₁₋₆alkyl substituted with a 5-9 membered heterocyclyl, wherein the 5-9 membered heterocyclyl is optionally substituted with one or more, identical or different R¹²,
each R¹² is selected from the group consisting of C₁₋₆alkyl, C₁₋₆alkoxy, halogen and 5-6 membered heterocyclyl.

In another aspect, the invention relates to the compound of the invention, or a salt thereof, wherein R⁵ is selected from the group consisting of

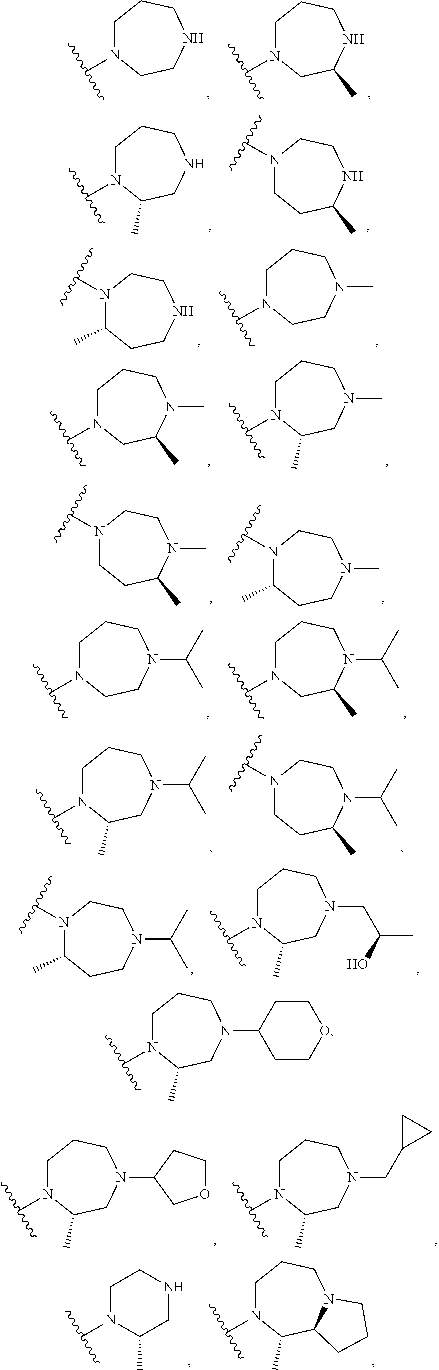

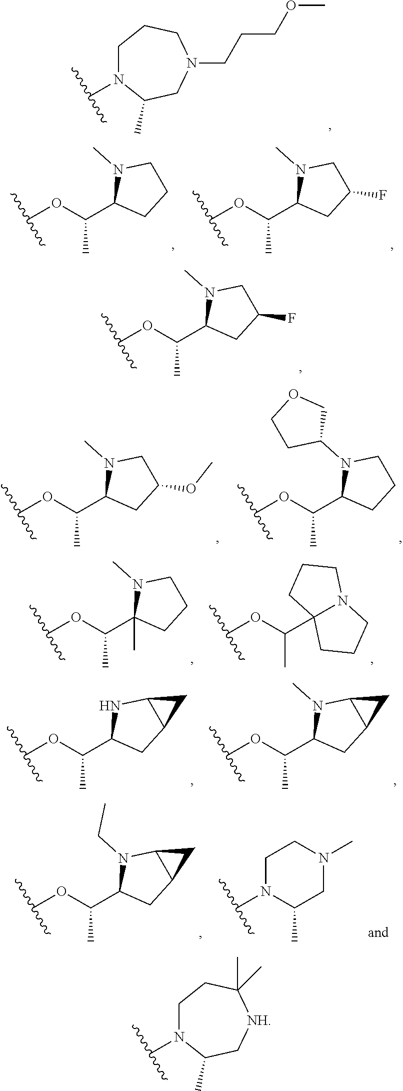

In another aspect, the invention relates to the compound of the invention, or a salt thereof, wherein R⁵ is selected from the group consisting of

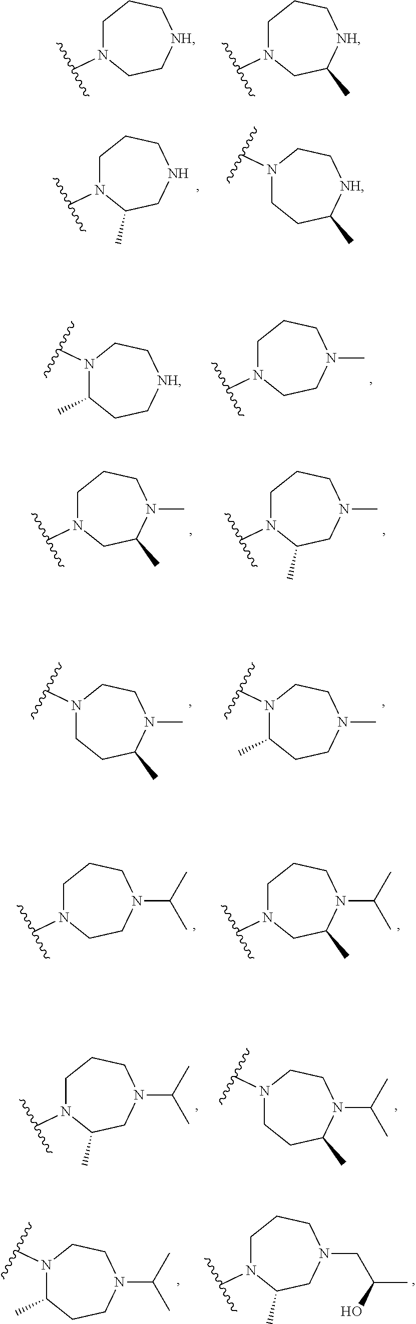

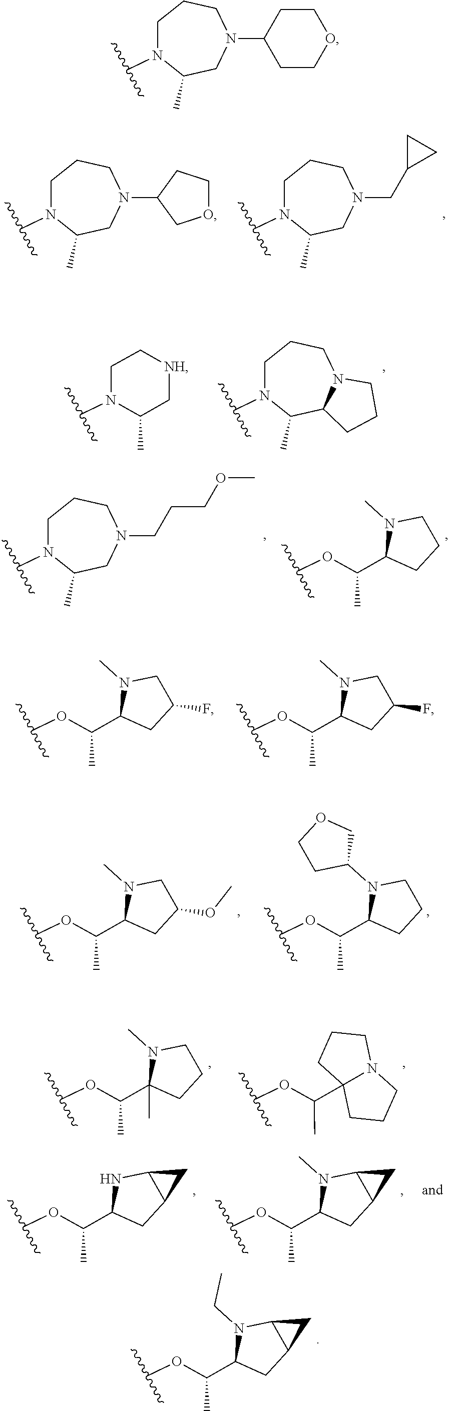

In another aspect, the invention relates to the compound of the invention, or a salt thereof, wherein R⁵ is selected from the group consisting of

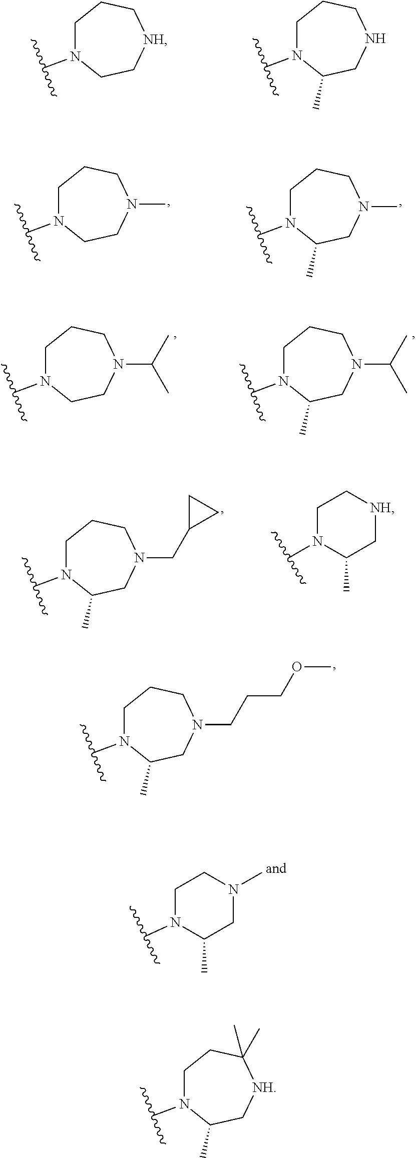

In another aspect, the invention relates to the compound of the invention, or a salt thereof, wherein R⁵ is selected from the group consisting of

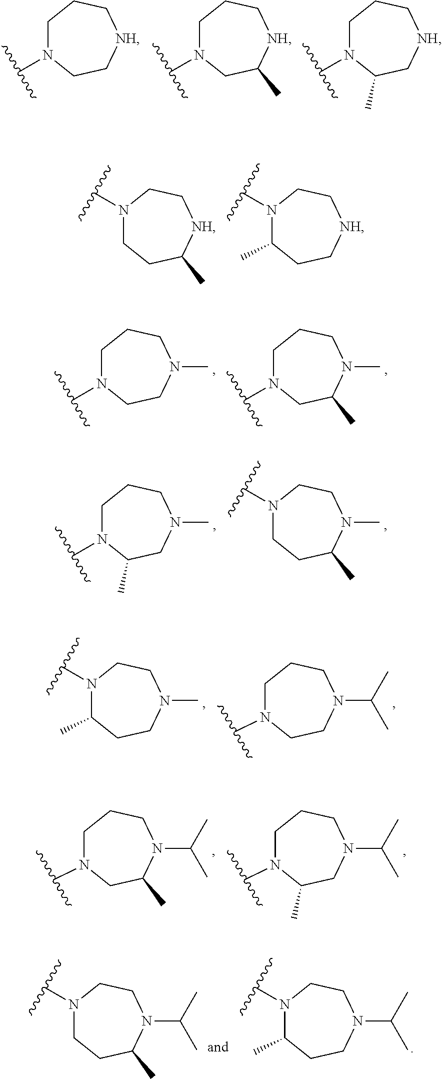

In another aspect, the invention relates to the compound of invention, or a salt thereof, wherein R⁵ is selected from the group consisting of

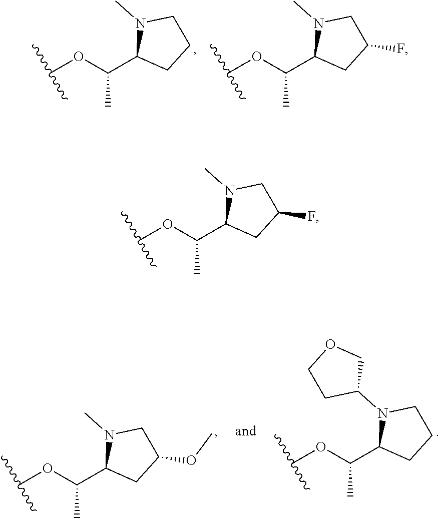

In another aspect, the invention relates to the compound of invention, or a salt thereof, wherein R⁵ is

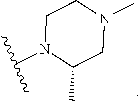 [SMILES: *N1CCN(C)C[C@@H]1C]

In another aspect, the invention relates to the compound of invention, or a salt thereof, wherein R⁵ is

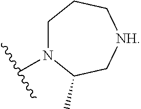 [SMILES: *N1CCCNC[C@@H]1C]

In another aspect, the invention relates to the compound of invention, or a salt thereof, wherein R⁵ is

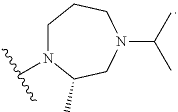 [SMILES: *N1CCCN(C(C)C)C[C@@H]1C]

In another aspect the invention relates to the compound of invention, or a salt thereof, wherein R⁵ is

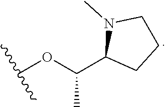 [SMILES: *O[C@@H](C)[C@@H]1CCCN1C]

In another aspect, the invention relates to the compound of invention, or a salt thereof, wherein R⁵ is selected from the group consisting of:

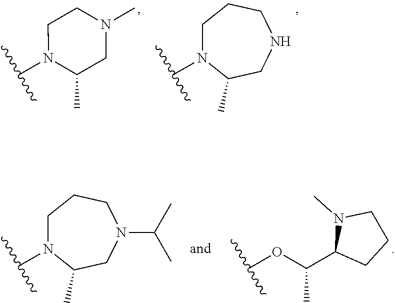

Preferred embodiments of the invention are example compounds V-1 to V-27 and any subset thereof.

Preferred embodiments of the invention are example compounds V-1 to V-11, V-24 to V-27 and any subset thereof.

Preferred embodiments of the invention are example compounds V-1 to V-11, V-24 to V-27 and any subset thereof.

It is to be understood that any two or more aspects and/or preferred embodiments of formula (V), (V*) (V′), (V″), (Va), (Vb), (Vc), (Vd), (Ve), (Vf), (Vg), (Vh), (Vi), (Vj)—or subformulas thereof—may be combined in any way leading to a chemically stable structure to obtain further aspects and/or preferred embodiments of formula (V), (V*) (V′), (V″), (Va), (Vb), (Vc), (Vd), (Ve), (Vf), (Vg), (Vh), (Vi), (Vj)—or subformulas thereof.

The present invention further relates to hydrates, solvates, polymorphs, metabolites, derivatives, stereoisomers and prodrugs of a compound of the invention (including all its embodiments).

The present invention further relates to a hydrate of a compound of the invention (including all its embodiments).

The present invention further relates to a solvate of a compound of the invention (including all its embodiments).

Compounds of the invention (including all its embodiments) which e.g. bear ester groups are potential prodrugs the ester being cleaved under physiological conditions and are also part of the invention.

The present invention further relates to a pharmaceutically acceptable salt of a compound of the invention (including all its embodiments).

The present invention further relates to a pharmaceutically acceptable salt of a compound of the invention (including all its embodiments) with anorganic or organic acids or bases.

### Pharmaceutical Compositions

A further object of the invention is a pharmaceutical composition comprising a compound of the invention—or a pharmaceutically acceptable salt thereof—and one or more pharmaceutically acceptable excipient(s).

In one aspect, said pharmaceutical composition optionally comprises one or more other pharmacologically active substance(s). Said one or more other pharmacologically active substance(s) may be the pharmacologically active substances or combination partners as herein defined.

Suitable pharmaceutical compositions for administering the compounds according to the invention will be apparent to those with ordinary skill in the art and include for example tablets, pills, capsules, suppositories, lozenges, troches, solutions, suspensions—particularly solutions, suspensions or other mixtures for injection (s.c., i.v., i.m.) and infusion (injectables)—elixirs, syrups, sachets, emulsions, inhalatives or dispersible powders. The content of the compounds of the invention should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day. Suitable tablets may be obtained, for example, by mixing the compound(s) the invention with known pharmaceutically acceptable excipients, for example inert diluents, carriers, disintegrants, adjuvants, surfactants, binders and/or lubricants. The tablets may also comprise several layers.

Coated tablets may be prepared accordingly by coating cores produced analogously to the tablets with excipients normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups or elixirs containing one or more compound(s) of the invention or combinations with one or more other pharmaceutically active substance(s) may additionally contain excipients like a sweetener such as saccharine, cyclamate, glycerol or sugar and a flavour enhancer, e.g. a flavouring such as vanillin or orange extract. They may also contain excipients like suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of excipients like isotonic agents, preservatives such as p-hydroxybenzoates, or stabilisers such as alkali metal salts of ethylenediamine tetra acetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more compound(s) of the invention or combinations with one or more other pharmaceutically active substance(s) may for example be prepared by mixing the compounds/active substance(s) with inert excipients such as lactose or sorbitol and packing them into gelatine capsules.

Suitable suppositories may be made for example by mixing with excipients provided for this purpose such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulfate).

The pharmaceutical compositions are administered by the usual methods, preferably by oral or transdermal route, most preferably by oral route. For oral administration the tablets may of course contain, apart from the above-mentioned excipients, additional excipients such as sodium citrate, calcium carbonate and dicalcium phosphate together with various excipients such as starch, preferably potato starch, gelatine and the like. Moreover, lubricants such as magnesium stearate, sodium lauryl sulfate and talc may be used at the same time for the tabletting process. In the case of aqueous suspensions, the active substances may be combined with various flavour enhancers or colourings in addition to the excipients mentioned above.

For parenteral use, solutions of the active substances with suitable liquid excipients may be used.

The dosage range of compound(s) of the invention applicable per day is usually from 1 mg to 2000 mg, preferably from 250 to 1250 mg.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts, it may be advisable to divide them up into a number of smaller doses spread over the day.

Thus, in a further aspect the invention relates to a pharmaceutical composition comprising at least one (preferably one) compound(s) of the invention—or a pharmaceutically acceptable salt thereof—and one or more pharmaceutically acceptable excipient(s).

The compounds of the invention—or the pharmaceutically acceptable salts thereof—and the pharmaceutical compositions comprising such compound and salts may also be co-administered with other pharmacologically active substances, e.g. with other anti-neoplastic compounds (e.g. chemotherapy), i.e. used in combination (see combination treatment further below).

The elements of such combinations may be administered (whether dependently or independently) by methods customary to the skilled person and as they are used in monotherapy, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable excipients appropriate for each route of administration.

The combinations may be administered at therapeutically effective single or divided daily doses. The active components of the combinations may be administered in such doses which are therapeutically effective in monotherapy, or in such doses which are lower than the doses used in monotherapy, but when combined result in a desired (joint) therapeutically effective amount.

However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmacological or therapeutic effect.

Thus, in a further aspect the invention also relates to a pharmaceutical composition comprising a compound of the invention—or a pharmaceutically acceptable salt thereof—and one or more (preferably one or two, most preferably one) other pharmacologically active substance(s).

In a further aspect the invention also relates to a pharmaceutical preparation comprising a compound of the invention—or a pharmaceutically acceptable salt thereof—and one or more (preferably one or two, most preferably one) other pharmacologically active substance(s).

Pharmaceutical compositions to be co-administered or used in combination can also be provided in the form of a kit.

Thus, in a further aspect the invention also relates to a kit comprising
a first pharmaceutical composition or dosage form comprising a compound of the invention and, optionally, one or more pharmaceutically acceptable excipient(s), and
a second pharmaceutical composition or dosage form comprising another pharmacologically active substance and, optionally, one or more pharmaceutically acceptable excipient(s).

In one aspect such kit comprises a third pharmaceutical composition or dosage form comprising still another pharmacologically active substance and, optionally, one or more pharmaceutically acceptable excipient(s).

### Medical Uses—Methods of Treatment

### Indications—Patient Populations

The present invention is directed to compounds inhibiting KRAS, preferably KRAS mutated at residue 12, such as KRAS G12C, KRAS G12D, KRAS G12V, KRAS G12A and KRAS G12R inhibitors, preferably inhibitors of KRAS G12C and/or KRAS G12D, or inhibitors selective for KRAS G12D, as well as compounds inhibiting KRAS wildtype, preferably amplified, KRAS mutated at residue 13, such as KRAS G13D, or KRAS mutated at residue 61, such as KRAS Q61H. In particular, compounds of the invention (including all embodiments thereof) are potentially useful in the treatment and/or prevention of diseases and/or conditions mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D, or by KRAS mutated at residue 61, such as KRAS Q61H.

Thus, in a further aspect the invention relates to a compound of the invention—or a pharmaceutically acceptable salt thereof—for use as a medicament.

In a further aspect the invention relates to a compound of the invention—or a pharmaceutically acceptable salt thereof—for use in a method of treatment of the human or animal body.

In a further aspect the invention relates to a compound of the invention—or a pharmaceutically acceptable salt thereof—for use in the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D.

In a further aspect the invention relates to the use of a compound of the invention—or a pharmaceutically acceptable salt thereof—in the manufacture of a medicament for the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D.

In a further aspect the invention relates to a method for the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D comprising administering a therapeutically effective amount of a compound of the invention—or a pharmaceutically acceptable salt thereof—to a human being.

In a further aspect the invention relates to a compound of the invention—or a pharmaceutically acceptable salt thereof—for use in the treatment and/or prevention of cancer.

In a further aspect the invention relates to a compound of the invention—or a pharmaceutically acceptable salt thereof—for use in a method of treatment and/or prevention of cancer in the human or animal body.

In a further aspect the invention relates to the use of a compound of the invention—or a pharmaceutically acceptable salt thereof—in the manufacture of a medicament for the treatment and/or prevention of cancer.

In a further aspect the invention relates to a method for the treatment and/or prevention of cancer comprising administering a therapeutically effective amount of a compound of the invention—or a pharmaceutically acceptable salt thereof—to a human being.

Preferably, the cancer as defined herein (above or below) comprises a KRAS mutation. In particular, KRAS mutations include e.g. mutations of the KRAS gene and of the KRAS protein, such as overexpressed KRAS, amplified KRAS or KRAS, KRAS mutated at residue 12, KRAS mutated at residue 13, KRAS mutated at residue 61, KRAS mutated at residue 146, in particular KRAS G12A, KRAS G12C, KRAS G12D, KRAS G12V, KRAS G12S, KRAS G13C, KRAS G13D, KRAS G13V, KRAS Q61H, KRAS Q61E, KRAS Q61P, KRAS A146P, KRAS A146T, KRAS A146V. KRAS may present one or more of these mutations/alterations.

Preferably, the cancer as defined herein (above or below) comprises a BRAF mutation in addition or in alternative to the KRAS mutation. Said BRAF mutation is in particular a class Ill BRAF mutation, e.g. as defined in Z. Yao, Nature, 2017, 548, 234-238.

Preferably, the cancer as defined herein (above or below) comprises a mutation in a receptor tyrosine kinase (RTK), including EGFR, MET and ERBB2 mutations, in addition or in alternative to the KRAS mutation.

In a further aspect the invention relates to a compound of the invention—or a pharmaceutically acceptable salt thereof—for use in the treatment and/or prevention of cancer, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to the use of a compound of the invention—or a pharmaceutically acceptable salt thereof—in the manufacture of a medicament for the treatment and/or prevention of cancer, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to a method for the treatment and/or prevention of cancer comprising administering a therapeutically effective amount of a compound of the invention—or a pharmaceutically acceptable salt thereof—to a human being, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to a compound of the invention—or a pharmaceutically acceptable salt thereof—for use in the treatment and/or prevention of cancer, wherein the cancer comprises a KRAS G12D mutation.

In a further aspect the invention relates to a compound of the invention—or a pharmaceutically acceptable salt thereof—for use in the treatment and/or prevention of cancer, wherein the cancer comprises a KRAS G12V mutation.

In a further aspect the invention relates to a compound of the invention—or a pharmaceutically acceptable salt thereof—for use in the treatment and/or prevention of cancer, wherein the cancer comprises a KRAS G13D mutation.

In a further aspect the invention relates to a compound of the invention—or a pharmaceutically acceptable salt thereof—for use in the treatment and/or prevention of cancer, wherein the cancer comprises wildtype amplified KRAS.

Another aspect is based on identifying a link between the KRAS status of a patient and potential susceptibility to treatment with a compound of the invention. A KRAS inhibitor, such as a compound of the invention, may then advantageously be used to treat patients with a disease dependent on KRAS who may be resistant to other therapies. This therefore provides opportunities, methods and tools for selecting patients for treatment with a compound of the invention particularly cancer patients. The selection is based on whether the tumor cells to be treated possess wild-type, preferably amplified, or KRAS mutated at residue 12, preferably G12C, G12D or G12V gene, or KRAS mutated at residue 13, preferably G13D gene. The KRAS gene status could therefore be used as a biomarker to indicate that selecting treatment with a compound of the invention may be advantageous.

According to one aspect, there is provided a method for selecting a patient for treatment with a compound of the invention the method comprising
providing a tumor cell-containing sample from a patient;
determining whether the KRAS gene in the patient's tumor cell-containing sample encodes for wild-type (glycine at position 12) or mutant (cysteine, aspartic acid, valine, alanine or arginine at position 12, aspartic acid at position 13, amplification and/or overexpression) KRAS protein; and
selecting a patient for treatment with a compound of the invention based thereon.

The method may include or exclude the actual patient sample isolation step.

According to another aspect, there is provided a compound of the invention—or a pharmaceutically acceptable salt thereof—for use in treating a cancer with tumor cells harbouring a KRAS mutation or an amplification of KRAS wildtype.

According to another aspect, there is provided a compound of the invention—or a pharmaceutically acceptable salt thereof—for use in treating a cancer with tumor cells harbouring a G12C mutant, G12D mutant, G12V mutant, G12A mutant, G13D mutant or G12R mutant KRAS gene or an amplification of KRAS wildtype.

According to another aspect, there is provided a compound of the invention—or a pharmaceutically acceptable salt thereof—for use in treating a cancer with tumor cells harbouring a G12C mutant, G12D mutant, G12V mutant or G13D mutant KRAS gene or an amplification of KRAS wildtype.

According to another aspect, there is provided a compound of the invention—or a pharmaceutically acceptable salt thereof—for use in treating a cancer with tumor cells harbouring a G12D mutant KRAS gene.

According to another aspect, there is provided a compound of the invention—or a pharmaceutically acceptable salt thereof—for use in treating a cancer with tumor cells harbouring a G12V mutant KRAS gene.

According to another aspect, there is provided a compound of the invention—or a pharmaceutically acceptable salt thereof—for use in treating a cancer with tumor cells harbouring a G13D mutant KRAS gene.

According to another aspect, there is provided a compound of the invention—or a pharmaceutically acceptable salt thereof—for use in treating a cancer with tumor cells harbouring wildtype amplified KRAS or overexpressed KRAS.

According to another aspect, there is provided a method of treating a cancer with tumor cells harbouring a G12C mutant, G12D mutant, G12V mutant, G12A mutant, G13D mutant or G12R mutant KRAS gene or an amplification of KRAS wildtype gene comprising administering an effective amount of a compound of the invention—or a pharmaceutically acceptable salt thereof—to a human being.

According to another aspect, there is provided a method of treating a cancer with tumor cells harbouring a G12C mutant, G12D mutant, G12V mutant, G12A mutant, G13D mutant or G12R mutant KRAS gene or an amplification of KRAS wildtype gene comprising administering an effective amount of a compound of the invention—or a pharmaceutically acceptable salt thereof.

Determining whether a tumor or cancer comprises a G12C KRAS mutation can be undertaken by assessing the nucleotide sequence encoding the KRAS protein, by assessing the amino acid sequence of the KRAS, protein, or by assessing the characteristics of a putative KRAS mutant protein. The sequence of wild-type human KRAS is known in the art. Methods for detecting a mutation in a KRAS nucleotide sequence are known by those of skill in the art.

These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS mutations by real-time PCR. In real-time PCR, fluorescent probes specific for the KRAS G12C mutation are used. When a mutation is present, the probe binds and fluorescence is detected. In some embodiments, the KRAS G12C mutation is identified using a direct sequencing method of specific regions (e.g. exon 2 and/or exon 3) in the KRAS gene. This technique will identify all possible mutations in the region sequenced. Methods for detecting a mutation in a KRAS protein are known by those of skill in the art. These methods include, but are not limited to, detection of a KRAS mutant using a binding agent (e.g. an antibody) specific for the mutant protein, protein electrophoresis, Western blotting and direct peptide sequencing.

Methods for determining whether a tumor or cancer comprises a G12C KRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA. In some embodiments the sample is a liquid biopsy and the test is done on a sample of blood to look for cancer cells from a tumor that are circulating in the blood or for pieces of DNA from tumor cells that are in the blood.

Analogously it can be determined whether a tumor or cancer comprises a KRAS G12D, KRAS G12V, KRAS G12A, KRAS G13D and KRAS G12R mutation or is a KRAS wildtype, preferably amplified.

Preferably, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, gastroesophageal cancer, chronic lymphocytic leukaemia, hepatocellular cancer, breast cancer, ovarian cancer, prostate cancer, glioblastoma, renal cancer and sarcomas.

Preferably, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of: pancreatic cancer, lung cancer, ovarian cancer, colorectal cancer (CRC), gastric cancer, gastroesophageal junction cancer (GEJC) and esophageal cancer.

In another aspect, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of pancreatic cancer (preferably pancreatic ductal adenocarcinoma (PDAC)), lung cancer (preferably non-small cell lung cancer (NSCLC)), gastric cancer, cholangiocarcinoma and colorectal cancer (preferably colorectal adenocarcinoma). Preferably, said pancreatic cancer, lung cancer, cholangiocarcinoma, colorectal cancer (CRC), pancreatic ductal adenocarcinoma (PDAC), non-small cell lung cancer (NSCLC) or colorectal adenocarcinoma comprises a KRAS mutation, in particular a KRAS G12D or KRAS G12V mutation. Preferably (in alternative or in combination with the previous preferred embodiment), said non-small cell lung cancer (NSCLC) comprises a mutation (in particular a loss-of-function mutation) in the NF1 gene.

In another aspect, the disease/condition/cancer/tumors/cancer cells to be treated/prevented with a compound of the invention—or a pharmaceutically acceptable salt thereof—according to the methods and uses as herein (above and below) defined and disclosed is gastric cancer, ovarian cancer or esophageal cancer, said gastric cancer or esophageal cancer being preferably selected from the group consisting of: gastric adenocarcinoma (GAC), esophageal adenocarcinoma (EAC) and gastroesophageal junction cancer (GEJC). Preferably, said gastric cancer, ovarian cancer, esophageal cancer, gastric adenocarcinoma (GAC), esophageal adenocarcinoma (EAC) or gastroesophageal junction cancer (GEJC) comprises a KRAS mutation or wildtype amplified KRAS.

Particularly preferred, the cancer to be treated/prevented with a compound of the invention—or a pharmaceutically acceptable salt thereof—according to the methods and uses as herein (above and below) defined and disclosed is selected from the group consisting of:
lung adenocarcinoma (preferably non-small cell lung cancer (NSCLC)) harbouring a KRAS mutation at position 12 (preferably a G12C, G12D, G12V, G12A, G12R mutation), at position 13 (preferably G13D) or an amplification of KRAS wildtype;
colorectal adenocarcinoma harbouring a KRAS mutation at position 12 (preferably a G12C, G12D, G12V, G12A, G12R mutation), at position 13 (preferably G13D) or an amplification of KRAS wildtype;
pancreatic adenocarcinoma (preferably pancreatic ductal adenocarcinoma (PDAC)) harbouring a RAS mutation at position 12 (preferably a KRAS and preferably a G12C, G12D, G12V, G12A, G12R mutation), at position 13 (preferably G13D) or an amplification of KRAS wildtype.

Preferably, “cancer” as used herein (above or below) includes drug-resistant cancer and cancer that has failed one, two or more lines of mono- or combination therapy with one or more anti-cancer agents. In particular, “cancer” (and any embodiment thereof) refers to any cancer (especially the cancer species defined hereinabove and hereinbelow) that is resistant to treatment with a KRAS G12C inhibitor.

Different resistance mechanisms have already been reported. For example, the following articles describe resistance in patients following treatment with a KRAS G12C inhibitor: (i) Awad M M, Liu S, Rybkin, I I, Arbour K C, Dilly J, Zhu V W, et al. Acquired resistance to KRAS(G12C) inhibition in cancer. N Engl J Med 2021; 384:2382-93 and (ii) Tanaka N, Lin J J, Li C, Ryan M B, Zhang J, Kiedrowski L A, et al. Clinical acquired resistance to KRAS(G12C) inhibition through a novel KRAS switch-II pocket mutation and polyclonal alterations converging on RAS-MAPK reactivation. Cancer Discov 2021; 11:1913-22.

In another aspect the disease/condition/cancer/tumors/cancer cells to be treated/prevented with a compound of the invention—or a pharmaceutically acceptable salt thereof—according to the methods and uses as herein (above and below) defined and disclosed is a RASopathy, preferably selected from the group consisting of Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

Additionally, the following cancers, tumors and other proliferative diseases may be treated with compounds of the invention—or a pharmaceutically acceptable salt thereof—without being restricted thereto. Preferably, the methods of treatment, methods, uses, compounds for use and pharmaceutical compositions for use as disclosed herein (above and below) are applied in treatments of diseases/conditions/cancers/tumors which (i.e. the respective cells) harbour a KRAS mutation at position 12 (preferably a G12C, G12D, G12V, G12A, G12R mutation) or an amplification of KRAS wildtype alternatively they have been identified to harbour a KRAS mutation at position 12 (preferably a G12C, G12D, G12V, G12A, G12R mutation) as herein described and/or referred or an amplification of KRAS wildtype: cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), gastroesophageal junction cancer pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis) gastric cancer;
cancers/tumors/carcinomas of the testis: e.g. seminomas, non-seminomas, gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);
All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithelial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas;
The compounds of the invention may be used in therapeutic regimens in the context of first line, second line, or any further line treatments.

The compounds of the invention may be used for the prevention, short-term or long-term treatment of the above-mentioned diseases/conditions/cancers/tumors, optionally also in combination with radiotherapy and/or surgery.

The methods of treatment, methods, uses and compounds for use as disclosed herein (above and below) can be performed with any compound of the invention—or a pharmaceutically acceptable salt thereof—as disclosed or defined herein and with any pharmaceutical composition or kit comprising a compound of the invention—or a pharmaceutically acceptable salt thereof (each including all individual embodiments or generic subsets of compounds of the invention.

### Combination Treatment

The compounds of the invention—or the pharmaceutically acceptable salts thereof—and the pharmaceutical compositions comprising such compounds or salts may also be co-administered with other pharmacologically active substances, e.g. with other anti-neoplastic compounds (e.g. chemotherapy), or used in combination with other treatments, such as radiation or surgical intervention, either as an adjuvant prior to surgery or post-operatively. Preferably, the pharmacologically active substance(s) for co-administration is/are (an) anti-neoplastic compound(s).

Thus, in a further aspect the invention relates to a compound of the invention—or a pharmaceutically acceptable salt thereof—for use as hereinbefore defined wherein said compound is administered before, after or together with one or more other pharmacologically active substance(s).

In a further aspect the invention relates to a compound of the invention—or a pharmaceutically acceptable salt thereof—for use as hereinbefore defined, wherein said compound is administered in combination with one or more other pharmacologically active substance(s).

In a further aspect the invention relates to the use of a compound of the invention—or a pharmaceutically acceptable salt thereof—as hereinbefore defined wherein said compound is to be administered before, after or together with one or more other pharmacologically active substance(s).

In a further aspect the invention relates to a method (e.g. a method for the treatment and/or prevention) as hereinbefore defined wherein the compound of the invention—or a pharmaceutically acceptable salt thereof—is administered before, after or together with a therapeutically effective amount of one or more other pharmacologically active substance(s).

In a further aspect the invention relates to a method (e.g. a method for the treatment and/or prevention) as hereinbefore defined wherein the compound of the invention—or a pharmaceutically acceptable salt thereof—is administered in combination with a therapeutically effective amount of one or more other pharmacologically active substance(s).

In a further aspect the invention relates to a method for the treatment and/or prevention of cancer comprising administering to a patient in need thereof a therapeutically effective amount of a compound of the invention—or a pharmaceutically acceptable salt thereof—and a therapeutically effective amount of one or more other pharmacologically active substance(s), wherein the compound of the invention—or a pharmaceutically acceptable salt thereof—is administered simultaneously, concurrently, sequentially, successively, alternately or separately with the one or more other pharmacologically active substance(s).

In a further aspect the invention relates to a method for the treatment and/or prevention of cancer comprising administering to a patient in need thereof a therapeutically effective amount of an inhibitor of a KRAS mutated at residue 12 or 13, such as KRAS G12C, KRAS G12D, KRAS G12V, KRAS G12A, KRAS G13D and/or KRAS G12R inhibitors, preferably KRAS G12C, KRAS G12D or selective KRAS G12D inhibitors—or a pharmaceutically acceptable salt thereof—and a therapeutically effective amount of one or more other pharmacologically active substance(s), wherein the inhibitor—or a pharmaceutically acceptable salt thereof—is administered in combination with the one or more other pharmacologically active substance(s).

In a further aspect the invention relates to a method for the treatment and/or prevention of cancer comprising administering to a patient in need thereof a therapeutically effective amount of an inhibitor of KRAS wildtype amplified or overexpressed—or a pharmaceutically acceptable salt thereof—and a therapeutically effective amount of one or more other pharmacologically active substance(s), wherein the inhibitor—or a pharmaceutically acceptable salt thereof—is administered in combination with the one or more other pharmacologically active substance(s).

In a further aspect the invention relates to a compound of the invention—or a pharmaceutically acceptable salt thereof—for use in the treatment and/or prevention of cancer, wherein the compound of the invention—or a pharmaceutically acceptable salt thereof—is administered simultaneously, concurrently, sequentially, successively, alternately or separately with the one or more other pharmacologically active substance(s).

In a further aspect the invention relates to an inhibitor of a KRAS mutated at residue 12 or 13, such as KRAS G12C, KRAS G12D, KRAS G12V, KRAS G12A, KRAS G13D and/or KRAS G12R inhibitors, preferably KRAS G12C, KRAS G12D or selective KRAS G12D inhibitors—or a pharmaceutically acceptable salt thereof—for use in the treatment and/or prevention of cancer, wherein the inhibitor—or a pharmaceutically acceptable salt thereof—is administered in combination with the one or more other pharmacologically active substance(s).

In a further aspect the invention relates to an inhibitor of KRAS wildtype amplified or overexpressed—or a pharmaceutically acceptable salt thereof—for use in the treatment and/or prevention of cancer, wherein the inhibitor—or a pharmaceutically acceptable salt thereof—is administered in combination with the one or more other pharmacologically active substance(s).

In a further aspect the invention relates to a kit comprising
a first pharmaceutical composition or dosage form comprising a compound of the invention—or a pharmaceutically acceptable salt thereof—and, optionally, one or more pharmaceutically acceptable excipient(s), and
a second pharmaceutical composition or dosage form comprising another pharmacologically active substance, and, optionally, one or more pharmaceutically acceptable excipient(s),
   for use in the treatment and/or prevention of cancer, wherein the first pharmaceutical composition is to be administered simultaneously, concurrently, sequentially, successively, alternately or separately with the second and/or additional pharmaceutical composition or dosage form.

In one aspect such kit for said use comprises a third pharmaceutical composition or dosage form comprising a third pharmaceutical composition or dosage form comprising still another pharmacologically active substance, and, optionally, one or more pharmaceutically acceptable excipient(s)

In a further embodiment of the invention the components (i.e. the combination partners) of the combinations, kits, uses, methods and compounds for use according to the invention (including all embodiments) are administered simultaneously.

In a further embodiment of the invention the components (i.e. the combination partners) of the combinations, kits, uses, methods and compounds for use according to the invention (including all embodiments) are administered concurrently.

In a further embodiment of the invention the components (i.e. the combination partners) of the combinations, kits, uses, methods and compounds for use according to the invention (including all embodiments) are administered sequentially.

In a further embodiment of the invention the components (i.e. the combination partners) of the combinations, kits, uses, methods and compounds for use according to the invention (including all embodiments) are administered successively.

In a further embodiment of the invention the components (i.e. the combination partners) of the combinations, kits, uses, methods and compounds for use according to the invention (including all embodiments) are administered alternately.

In a further embodiment of the invention the components (i.e. the combination partners) of the combinations, kits, uses, methods and compounds for use according to the invention (including all embodiments) are administered separately.

The pharmacologically active substance(s) to be used together/in combination with the compound of the invention—or a pharmaceutically acceptable salt thereof—(including all individual embodiments or generic subsets of compounds) or in the medical uses, uses, methods of treatment and/or prevention, pharmaceutical compositions as herein (above and below) defined can be selected from any one or more of the following (preferably there is one or two additional pharmacologically active substance used in all these embodiments):
1. an inhibitor of EGFR and/or ErbB2 (HER2) and/or ErbB3 (HER3) and/or ErbB4 (HER4) or of any mutants thereof
   a. irreversible inhibitors: e.g. afatinib, dacomitinib, canertinib, neratinib, avitinib, poziotinib, AV 412, PF-6274484, HKI 357, olmutinib, osimertinib, almonertinib, nazartinib, lazertinib, pelitinib;
   b. reversible inhibitors: e.g. erlotinib, gefitinib, icotinib, sapitinib, lapatinib, varlitinib, vandetanib, TAK-285, AEE788, BMS599626/AC-480, GW 583340;
   c. anti-EGFR antibodies: e.g. necitumumab, panitumumab, cetuximab, amivantamab;
   d. anti-HER2 antibodies: e.g. pertuzumab, trastuzumab, trastuzumab emtansine;
   e. inhibitors of mutant EGFR;
   f. an inhibitor of HER2 with exon 20 mutations;
   g. preferred irreversible inhibitor is afatinib;
   h. preferred anti-EGFR antibody is cetuximab.
2. an inhibitor of MEK and/or of mutants thereof
   a. e.g. trametinib, cobimetinib, binimetinib, selumetinib, refametinib;
   b. preferred is trametinib
   c. a MEK inhibitor as disclosed in WO 2013/136249;
   d. a MEK inhibitor as disclosed in WO 2013/136254
3. an inhibitor of SOS1 and/or of any mutants thereof (i.e. a compound that modulates/inhibits the GEF functionality of SOS1, e.g. by binding to SOS1 and preventing protein-protein interaction between SOS1 and a (mutant) Ras protein, e.g. KRAS)
   a. e.g. BAY-293;
   b. a SOS1 inhibitor as disclosed in WO 2018/115380;
   c. a SOS1 inhibitor as disclosed in WO 2019/122129;
   d. a SOS1 inhibitor as disclosed in WO 2020/180768, WO 2020/180770, WO 2018/172250 and WO 2019/201848.
4. an inhibitor of YAP1, WWTR1, TEAD1, TEAD2, TEAD3 and/or TEAD4
   a. reversible inhibitors of TEAD transcription factors (e.g. disclosed in WO 2018/204532);
   b. irreversible inhibitors of TEAD transcription factors (e.g. disclosed in WO 2020/243423);
   c. protein-protein interaction inhibitors of the YAP/TAZ::TEAD interaction (e.g. disclosed in WO 2021/186324);
   d. inhibitors of TEAD palmitoylation.
5. an oncolytic virus
6. a RAS vaccine
   a. e.g. TG02 (Targovax).
7. a cell cycle inhibitor
   a. e.g. inhibitors of CDK4/6 and/or of any mutants thereof
      i. e.g. palbociclib, ribociclib, abemaciclib, trilaciclib, PF-06873600;
      ii. preferred are palbociclib and abemaciclib;
      iii. most preferred is abemaciclib.
   b. e.g. vinca alkaloids
      i. e.g. vinorelbine.
   c. e.g. inhibitors of Aurora kinase and/or of any mutants thereof
      i. e.g. alisertib, barasertib.
8. an inhibitor of PTK2 (=FAK) and/or of any mutants thereof
   a. e.g. TAE226, BI 853520.
9. an inhibitor of SHP2 and/or of any mutants thereof
   a. e.g. SHP099, TNO155, RMC-4550, RMC-4630, IACS-13909.
10. an inhibitor of PI3 kinase (=PI3K) and/or of any mutants thereof
   a. e.g. inhibitors of PI3Kα and/or of any mutants thereof
      i. e.g. alpelisib, serabelisib, GDC-0077, HH-CYH33, AMG 511, buparlisib, dactolisib, pictilisib, taselisib.
11. an inhibitor of FGFR1 and/or FGFR2 and/or FGFR3 and/or of any mutants thereof
   a. e.g. ponatinib, infigratinib, nintedanib.
12. an inhibitor of AXL and/or of any mutants thereof
13. a taxane
   a. e.g. paclitaxel, nab-paclitaxel, docetaxel;
   b. preferred is paclitaxel.
14. a platinum-containing compound
   a. e.g. cisplatin, carboplatin, oxaliplatin
   b. preferred is oxaliplatin.
15. an anti-metabolite
   a. e.g. 5-fluorouracil, capecitabine, floxuridine, cytarabine, gemcitabine, pemetrexed, combination of trifluridine and tipiracil (=TAS102);
   b. preferred is 5-fluorouracil.
16. an immunotherapeutic agent
   a. e.g. an immune checkpoint inhibitor
      i. e.g. an anti-CTLA4 mAb, anti-PD1 mAb, anti-PD-L1 mAb, anti-PD-L2 mAb, anti-LAG3 mAb, anti-TIM3 mAb;
      ii. preferred is an anti-PD1 mAb;
      iii. e.g. ipilimumab, nivolumab, pembrolizumab, tislelizumab atezolizumab, avelumab, durvalumab, pidilizumab, PDR-001 (=spartalizumab), AMG-404, ezabenlimab;
      iv. preferred are nivolumab, pembrolizumab, ezabenlimab and PDR-001 (=spartalizumab);
      v. most preferred is ezabenlimab, pembrolizumab and nivolumab.
17. a topoisomerase inhibitor
   a. e.g. irinotecan, liposomal irinotecan (nal-IRI), topotecan, etoposide;
   b. most preferred is irinotecan and liposomal irinotecan (nal-IRI).
18. an inhibitor of A-Raf and/or B-Raf and/or C-Raf and/or of any mutants thereof
   a. e.g. encorafenib, dabrafenib, vemurafenib, PLX-8394, RAF-709 (=example 131 in WO 2014/151616), LXH254, sorafenib, LY-3009120 (=example 1 in WO 2013/134243), lifirafenib, TAK-632, agerafenib, CCT196969, RO5126766, RAF265.
19. an inhibitor of mTOR
   a. e.g. rapamycin, temsirolimus, everolimus, ridaforolimus, zotarolimus, sapanisertib, Torin 1, dactolisib, GDC-0349, VS-5584, vistusertib, AZD8055.
20. an epigenetic regulator
   a. e.g. a BET inhibitor
      i. e.g. JQ-1, GSK 525762, OTX-015, CPI-0610, TEN-010, OTX-015, PLX51107, ABBV-075, ABBV-744, BMS986158, TGI-1601, CC-90010, AZD5153, I-BET151, BI 894999;
21. an inhibitor of IGF1/2 and/or of IGF1-R and/or of any mutants thereof
   a. e.g. xentuzumab (antibody 60833 in WO 2010/066868), MEDI-573 (=dusigitumab), linsitinib.
22. an inhibitor of a Src family kinase and/or of any mutants thereof
   a. e.g. an inhibitor of a kinase of the SrcA subfamily and/or of any mutants thereof, i.e. an inhibitor of Src, Yes, Fyn, Fgr and/or of any mutants thereof;
   b. e.g. an inhibitor of a kinase of the SrcB subfamily and/or of any mutants thereof, i.e. an inhibitor of Lck, Hck, Blk, Lyn and/or of any mutants thereof;
   c. e.g. an inhibitor of a kinase of the Frk subfamily and/or of any mutants thereof, i.e. an inhibitor of Frk and/or of any mutants thereof;
   d. e.g. dasatinib, ponatinib, bosutinib, vandetanib, KX-01, saracatinib, KX2-391, SU 6656, WH-4-023.
23. an apoptosis regulator
   a. e.g. an MDM2 inhibitor, e.g. an inhibitor of the interaction between p53 (preferably functional p53, most preferably wt p53) and MDM2 and/or of any mutants thereof;
      i. e.g. HDM-201, NVP-CGM097, RG-7112, MK-8242, RG-7388, SAR405838, AMG-232, DS-3032, RG-7775, APG-115;
      ii. preferred are HDM-201, RG-7388 and AMG-232;
      iii. an MDM2 inhibitor as disclosed in WO 2015/155332;
      iv. an MDM2 inhibitor as disclosed in WO 2016/001376;
      v. an MDM2 inhibitor as disclosed in WO 2016/026937;
      vi. an MDM2 inhibitor as disclosed in WO 2017/060431;
   b. e.g. a PARP inhibitor;
   c. e.g. an MCL-1 inhibitor;
      i. e.g. AZD-5991, AMG-176, AMG-397, S64315, S63845, A-1210477;
24. an inhibitor of c-MET and/or of any mutants thereof
   a. e.g. savolitinib, cabozantinib, foretinib;
   b. MET antibodies, e.g. emibetuzumab, amivantamab;
25. an inhibitor of ERK and/or of any mutants thereof
   a. e.g. ulixertinib, LTT462;
26. an inhibitor of farnesyl transferase and/or of any mutants thereof
   a. e.g. tipifarnib;

In a further embodiment of the (combined) use and method (e.g. method for the treatment and/or prevention) as hereinbefore described one other pharmacologically active substance is to be administered before, after or together with the compound of the invention—or a pharmaceutically acceptable salt thereof—wherein said one other pharmacologically active substance is
a SOS1 inhibitor; or
a MEK inhibitor; or
trametinib, or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ::TEAD inhibitor.

In a further embodiment of the (combined) use and method (e.g. method for the treatment and/or prevention) as hereinbefore described one other pharmacologically active substance is to be administered in combination with the compound of the invention—or a pharmaceutically acceptable salt thereof—wherein said one other pharmacologically active substance is
a SOS1 inhibitor; or
a MEK inhibitor; or
trametinib; or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ::TEAD inhibitor.

In a further aspect of the (combined) use and method (e.g. method for the treatment and/or prevention) as hereinbefore described two other pharmacologically active substances are to be administered before, after or together with the compound of the invention—or a pharmaceutically acceptable salt thereof—wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and cetuximab; or
a SOS1 inhibitor and an inhibitor of TEAD palmitoylation; or
a SOS1 inhibitor and a YAP/TAZ::TEAD inhibitor.

In a further aspect of the (combined) use and method (e.g. method for the treatment and/or prevention) as hereinbefore described two other pharmacologically active substances are to be administered in combination with the compound of the invention—or a pharmaceutically acceptable salt thereof—wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and cetuximab; or
a SOS1 inhibitor and an inhibitor of TEAD palmitoylation; or
a SOS1 inhibitor and a YAP/TAZ::TEAD inhibitor.

Additional pharmacologically active substance(s) which can also be used together/in combination with the compound of the invention—or a pharmaceutically acceptable salt thereof—(including all individual embodiments or generic subsets of compounds of the invention or in the medical uses, uses, methods of treatment and/or prevention, pharmaceutical compositions, kits as herein (above and below) defined include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP inhibitors/SMAC mimetics, Mcl-1, MDM2/MDMX), MEK inhibitors, ERK inhibitors, FLT3 inhibitors, BRD4 inhibitors, IGF-1R inhibitors, TRAILR2 agonists, Bcl-xL inhibitors, Bcl-2 inhibitors (e.g. venetoclax), Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors (e.g. carfilzomib), immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs®) like e.g. CD3×BCMA, CD3×CD33, CD3×CD19), PSMA×CD3), tumor vaccines, immunomodulator, e.g. STING agonist, and various chemotherapeutic agents such as amifostin, anagrelid, clodronat, filgrastin, interferon, interferon alpha, leucovorin, procarbazine, levamisole, mesna, mitotane, pamidronate and porfimer.

It is to be understood that the combinations, compositions, kits, methods, uses, pharmaceutical compositions or compounds for use according to this invention may envisage the simultaneous, concurrent, sequential, successive, alternate or separate administration of the active ingredients or components. It will be appreciated that the compound of the invention—or a pharmaceutically acceptable salt thereof—and the one or more other pharmacologically active substance(s) can be administered formulated either dependently or independently, such as e.g. the compound of the invention—or a pharmaceutically acceptable salt thereof—and the one or more other pharmacologically active substance(s) may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are administered to a patient simultaneously in the form of a single entity or dosage. The term “non-fixed combination” means that the active ingredients are administered to a patient as separate entities either simultaneously, concurrently or sequentially with no specific time limits, wherein such administration provides therapeutically effective levels of the compounds in the body of the patient.

The administration of the compound of formula the invention—or a pharmaceutically acceptable salt thereof—and the one or more other pharmacologically active substance(s) may take place by co-administering the active components or ingredients, such as e.g. by administering them simultaneously or concurrently in one single or in two or more separate formulations or dosage forms. Alternatively, the administration of the compound of the invention—or a pharmaceutically acceptable salt thereof—and the one or more other pharmacologically active substance(s) may take place by administering the active components or ingredients sequentially or in alternation, such as e.g. in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to:

The use of the prefix C_{x-y}, wherein x and y each represent a positive integer (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (e.g. heteroaryl, heteroarylalkyl, heterocyclyl, heterocycylalkyl) relates to the total number of atoms of all the ring members or the total of all the ring and carbon chain members.

The indication of the number of carbon atoms in groups that consist of a combination of carbon chain and carbon ring structure (e.g. cycloalkylalkyl, arylalkyl) relates to the total number of carbon atoms of all the carbon ring and carbon chain members. Obviously, a ring structure has at least three members.

In general, for groups comprising two or more subgroups (e.g. heteroarylalkyl, heterocycylalkyl, cycloalkylalkyl, arylalkyl) the last named subgroup is the radical attachment point, for example, the substituent aryl-C₁₋₆alkyl means an aryl group which is bound to a C₁₋₆ alkyl group, the latter of which is bound to the core or to the group to which the substituent is attached.

In groups like HO, H₂N, (O)S, (O)₂S, NC (cyano), HOOC, F₃C or the like, the skilled artisan can see the radical attachment point(s) to the molecule from the free valences of the group itself.

The expression “compound of the invention” and grammatical variants thereof comprises compounds of formula (V), (V*) (V′), (V″), (Va), (Vb), (Vc), (Vd), (Ve), (Vf), (Vg), (Vh), (Vi), (Vj), including all salts, aspects and preferred embodiments thereof as herein defined. Any reference to a compound of the invention or to a compound of formula (V), (V*) (V′), (V″), (Va), (Vb), (Vc), (Vd), (Ve), (Vf), (Vg), (Vh), (Vi), (Vj), is intended to include a reference to the respective (sub)aspects and embodiments.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}alkylamino or C_{x-y}alkyloxy.

The term alkylene can also be derived from alkyl. Alkylene is bivalent, unlike alkyl, and requires two binding partners. Formally, the second valency is produced by removing a hydrogen atom in an alkyl. Corresponding groups are for example —CH₃ and —CH₂—, —CH₂CH₃ and —CH₂CH₂— or >CHCH₃ etc.

The term “C₁₋₄alkylene” includes for example —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CH(CH(CH₃))₂)— and —C(CH₃)(CH₂CH₃)—.

Other examples of alkylene are methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene, hexylene etc.

By the generic terms propylene, butylene, pentylene, hexylene etc. without any further definition are meant all the conceivable isomeric forms with the corresponding number of carbon atoms, i.e. propylene includes 1-methylethylene and butylene includes 1-methylpropylene, 2-methylpropylene, 1,1-dimethylethylene and 1,2-dimethylethylene.

The above definition for alkylene also applies if alkylene is part of another (combined) group such as for example in HO—C_{x-y}alkyleneamino or H₂N—C_{x-y}alkyleneoxy.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkylene, alkenylene consists of at least two carbon atoms, wherein at least two adjacent carbon atoms are joined together by a C—C double bond and a carbon atom can only be part of one C—C double bond. If in an alkylene as hereinbefore defined having at least two carbon atoms, two hydrogen atoms at adjacent carbon atoms are formally removed and the free valencies are saturated to form a second bond, the corresponding alkenylene is formed. Examples of alkenylene are ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene, hexenylene etc.

By the generic terms propenylene, butenylene, pentenylene, hexenylene etc. without any further definition are meant all the conceivable isomeric forms with the corresponding number of carbon atoms, i.e. propenylene includes 1-methylethenylene and butenylene includes 1-methylpropenylene, 2-methylpropenylene, 1,1-dimethylethenylene and 1,2-dimethylethenylene.

Alkenylene may optionally be present in the cis or trans or E or Z orientation with regard to the double bond(s).

The above definition for alkenylene also applies when alkenylene is a part of another (combined) group as for example in HO—C_{x-y}alkenyleneamino or H₂N—C_{x-y}alkenyleneoxy.

Unlike alkyl, alkynyl consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Examples of alkynyl are ethynyl, prop-1-ynyl, prop-2-ynyl, but-1-ynyl, but-2-ynyl, but-3-ynyl, 1-methyl-prop-2-ynyl, pent-1-ynyl, pent-2-ynyl, pent-3-ynyl, pent-4-ynyl, 3-methyl-but-1-ynyl, hex-1-ynyl, hex-2-ynyl, hex-3-ynyl, hex-4-ynyl, hex-5-ynyl etc.

By the generic terms propynyl, butynyl, pentynyl, hexynyl, heptynyl, octynyl, nonynyl, decynyl etc. without any further definition are meant all the conceivable isomeric forms with the corresponding number of carbon atoms, i.e. propynyl includes prop-1-ynyl and prop-2-ynyl, butynyl includes but-1-ynyl, but-2-ynyl, but-3-ynyl, 1-methyl-prop-1-ynyl, 1-methyl-prop-2-ynyl, etc.

If a hydrocarbon chain carries both at least one double bond and also at least one triple bond, by definition it belongs to the alkynyl subgroup.

The above definition for alkynyl also applies if alkynyl is part of another (combined) group, as for example in C_{x-y}alkynylamino or C_{x-y}alkynyloxy.

Unlike alkylene, alkynylene consists of at least two carbon atoms, wherein at least two adjacent carbon atoms are joined together by a C—C triple bond. If in an alkylene as hereinbefore defined having at least two carbon atoms, two hydrogen atoms in each case at adjacent carbon atoms are formally removed and the free valencies are saturated to form two further bonds, the corresponding alkynylene is formed.

Examples of alkynylene are ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene, hexynylene etc.

By the generic terms propynylene, butynylene, pentynylene, hexynylene etc. without any further definition are meant all the conceivable isomeric forms with the corresponding number of carbon atoms, i.e. propynylene includes 1-methylethynylene and butynylene includes 1-methylpropynylene, 2-methylpropynylene, 1,1-dimethylethynylene and 1,2-dimethylethynylene.

The above definition for alkynylene also applies if alkynylene is part of another (combined) group, as for example in HO—C_{x-y}alkynyleneamino or H₂N—C_{x-y}alkynyleneoxy.

By heteroatoms are meant oxygen, nitrogen and sulphur atoms.

Haloalkyl (haloalkenyl, haloalkynyl) is derived from the previously defined alkyl (alkenyl, alkynyl) by replacing one or more hydrogen atoms of the hydrocarbon chain independently of one another by halogen atoms, which may be identical or different. If a haloalkyl (haloalkenyl, haloalkynyl) is to be further substituted, the substitutions may take place independently of one another, in the form of mono- or polysubstitutions in each case, on all the hydrogen-carrying carbon atoms.

Examples of haloalkyl (haloalkenyl, haloalkynyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —C≡C—CF₃, —CHFCH₂CH₃, —CHFCH₂CF₃ etc.

From the previously defined haloalkyl (haloalkenyl, haloalkynyl) are also derived the terms haloalkylene (haloalkenylene, haloalkynylene). Haloalkylene (haloalkenylene, haloalkynylene), unlike haloalkyl (haloalkenyl, haloalkynyl), is bivalent and requires two binding partners. Formally, the second valency is formed by removing a hydrogen atom from a haloalkyl (haloalkenyl, haloalkynyl).

Corresponding groups are for example —CH₂F and —CHF—, —CHFCH₂F and —CHFCHF— or >CFCH₂F etc.

The above definitions also apply if the corresponding halogen-containing groups are part of another (combined) group.

Halogen denotes fluorine, chlorine, bromine and/or iodine atoms.

Cycloalkyl is made up of the subgroups monocyclic cycloalkyl, bicyclic cycloalkyl and spiro-cycloalkyl. The ring systems are saturated and formed by linked carbon atoms. In bicyclic cycloalkyl two rings are joined together so that they have at least two carbon atoms in common. In spiro-cycloalkyl one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

The above definition for cycloalkyl also applies if cycloalkyl is part of another (combined) group as for example in C_{x-y}cycloalkylamino, C_{x-y}cycloalkyloxy or C_{x-y}cycloalkylalkyl.

If the free valency of a cycloalkyl is saturated, then an alicycle is obtained.

The term cycloalkylene can thus be derived from the previously defined cycloalkyl. Cycloalkylene, unlike cycloalkyl, is bivalent and requires two binding partners. Formally, the second valency is obtained by removing a hydrogen atom from a cycloalkyl. Corresponding groups are for example: cyclohexyl and

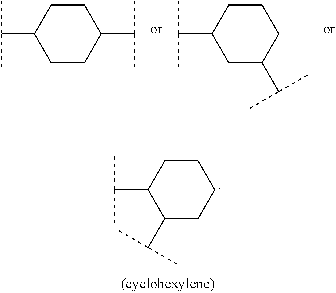

The above definition for cycloalkylene also applies if cycloalkylene is part of another (combined) group as for example in HO—C_{x-y}cycloalkyleneamino or H₂N—C_{x-y}cycloalkyleneoxy.

Cycloalkenyl is made up of the subgroups monocyclic cycloalkenyl, bicyclic cycloalkenyl and spiro-cycloalkenyl. However, the systems are unsaturated, i.e. there is at least one C—C double bond but no aromatic system. If in a cycloalkyl as hereinbefore defined two hydrogen atoms at adjacent cyclic carbon atoms are formally removed and the free valencies are saturated to form a second bond, the corresponding cycloalkenyl is obtained.

If a cycloalkenyl is to be substituted, the substitutions may take place independently of one another, in the form of mono- or polysubstitutions in each case, on all the hydrogen-carrying carbon atoms. Cycloalkenyl itself may be linked as a substituent to the molecule via every suitable position of the ring system.

Examples of cycloalkenyl are cycloprop-1-enyl, cycloprop-2-enyl, cyclobut-1-enyl, cyclobut-2-enyl, cyclopent-1-enyl, cyclopent-2-enyl, cyclopent-3-enyl, cyclohex-1-enyl, cyclohex-2-enyl, cyclohex-3-enyl, cyclohept-1-enyl, cyclohept-2-enyl, cyclohept-3-enyl, cyclohept-4-enyl, cyclobuta-1,3-dienyl, cyclopenta-1,4-dienyl, cyclopenta-1,3-dienyl, cyclopenta-2,4-dienyl, cyclohexa-1,3-dienyl, cyclohexa-1,5-dienyl, cyclohexa-2,4-dienyl, cyclohexa-1,4-dienyl, cyclohexa-2,5-dienyl, bicyclo[2.2.1]hepta-2,5-dienyl (norborna-2,5-dienyl), bicyclo[2.2.1]hept-2-enyl (norbornenyl), spiro[4,5]dec-2-enyl etc.

The above definition for cycloalkenyl also applies when cycloalkenyl is part of another (combined) group as for example in C_{x-y}cycloalkenylamino, C_{x-y}cycloalkenyloxy or C_{x-y}cycloalkenylalkyl.

If the free valency of a cycloalkenyl is saturated, then an unsaturated alicycle is obtained. The term cycloalkenylene can thus be derived from the previously defined cycloalkenyl. Cycloalkenylene, unlike cycloalkenyl, is bivalent and requires two binding partners. Formally, the second valency is obtained by removing a hydrogen atom from a cycloalkenyl. Corresponding groups are for example: cyclopentenyl and

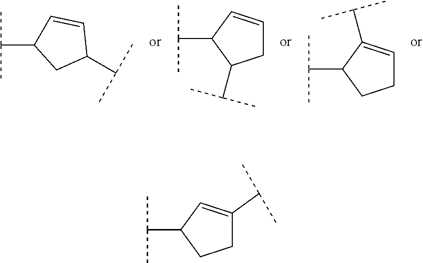

(cyclopentenylene) etc.

The above definition for cycloalkenylene also applies if cycloalkenylene is part of another (combined) group as for example in HO—C_{x-y}cycloalkenyleneamino or H₂N—C_{x-y}cycloalkenyleneoxy.

Aryl denotes mono-, bi- or tricyclic carbocycles with at least one aromatic carbocycle.

Preferably, it denotes a monocyclic group with six carbon atoms (phenyl) or a bicyclic group with nine or ten carbon atoms (two six-membered rings or one six-membered ring with a five-membered ring), wherein the second ring may also be aromatic or, however, may also be partially saturated.

If an aryl is to be substituted, the substitutions may take place independently of one another, in the form of mono- or polysubstitutions in each case, on all the hydrogen-carrying carbon atoms. Aryl itself may be linked as a substituent to the molecule via every suitable position of the ring system.

Examples of aryl are phenyl, naphthyl, indanyl (2,3-dihydroindenyl), indenyl, anthracenyl, phenanthrenyl, tetrahydronaphthyl (1,2,3,4-tetrahydronaphthyl, tetralinyl), dihydronaphthyl (1,2-dihydronaphthyl), fluorenyl etc. Most preferred is phenyl.

The above definition of aryl also applies if aryl is part of another (combined) group as for example in arylamino, aryloxy or arylalkyl.

If the free valency of an aryl is saturated, then an aromatic group is obtained.

The term arylene can also be derived from the previously defined aryl. Arylene, unlike aryl, is bivalent and requires two binding partners. Formally, the second valency is formed by removing a hydrogen atom from an aryl. Corresponding groups are for example:

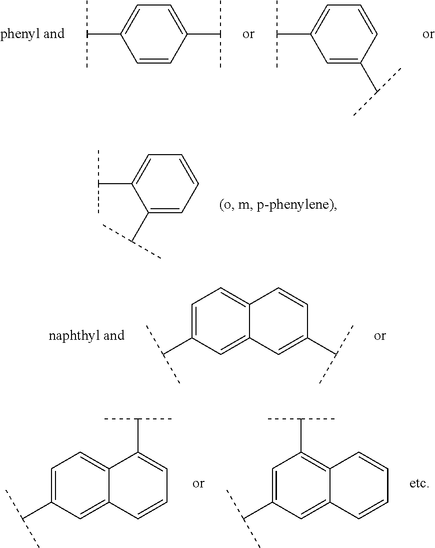

The above definition for arylene also applies if arylene is part of another (combined) group as for example in HO-aryleneamino or H₂N-aryleneoxy.

Heterocyclyl denotes ring systems, which are derived from the previously defined cycloalkyl, cycloalkenyl and aryl by replacing one or more of the groups —CH₂— independently of one another in the hydrocarbon rings by the groups —O—, —S— or —NH— or by replacing one or more of the groups ═CH— by the group ═N—, wherein a total of not more than five heteroatoms may be present, at least one carbon atom must be present between two oxygen atoms and between two sulphur atoms or between an oxygen and a sulphur atom and the ring as a whole must have chemical stability. Heteroatoms may optionally be present in all the possible oxidation stages (sulphur→sulfoxide —SO—, sulphone —SO₂—; nitrogen→N-oxide). In a heterocyclyl there is no heteroaromatic ring, i.e. no heteroatom is part of an aromatic system. A direct result of the derivation from cycloalkyl, cycloalkenyl and aryl is that heterocyclyl is made up of the subgroups monocyclic heterocyclyl, bicyclic heterocyclyl, tricyclic heterocyclyl and spiro-heterocyclyl, which may be present in saturated or unsaturated form. By unsaturated is meant that there is at least one double bond in the ring system in question, but no heteroaromatic system is formed. In bicyclic heterocyclyl two rings are linked together so that they have at least two (hetero)atoms in common. In spiro-heterocyclyl one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system. Substituents on heterocyclyl do not count for the number of members of a heterocyclyl.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diaza-spiro[4,5]decyl etc.

Further examples are the structures illustrated below, which may be attached via each hydrogen-carrying atom (exchanged for hydrogen):

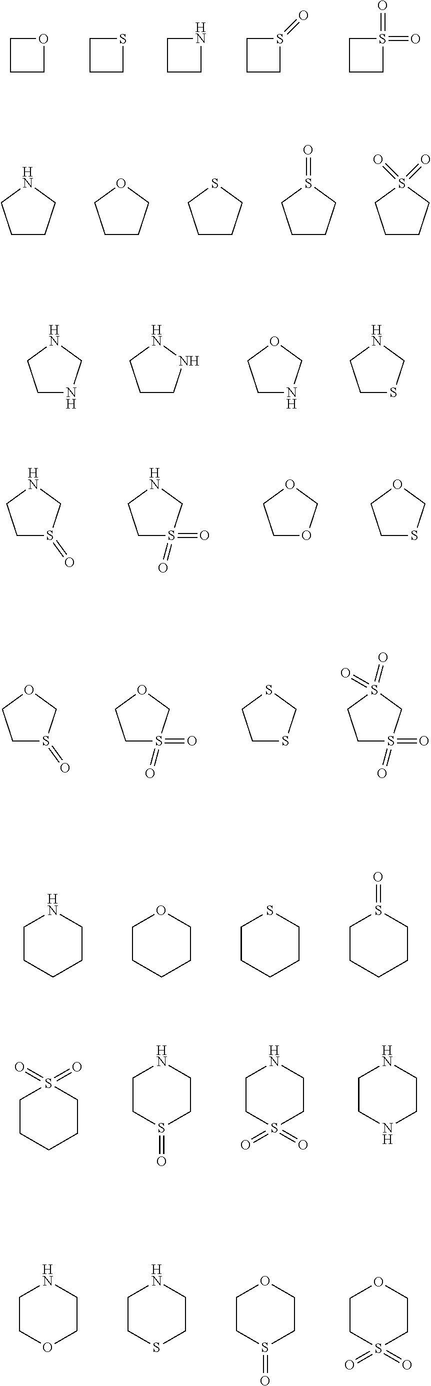

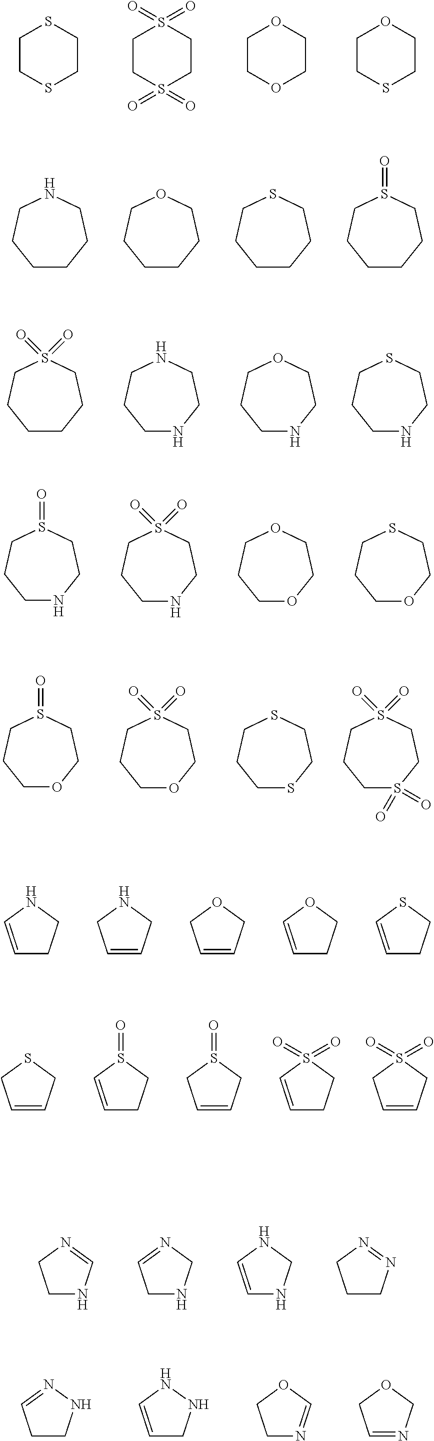

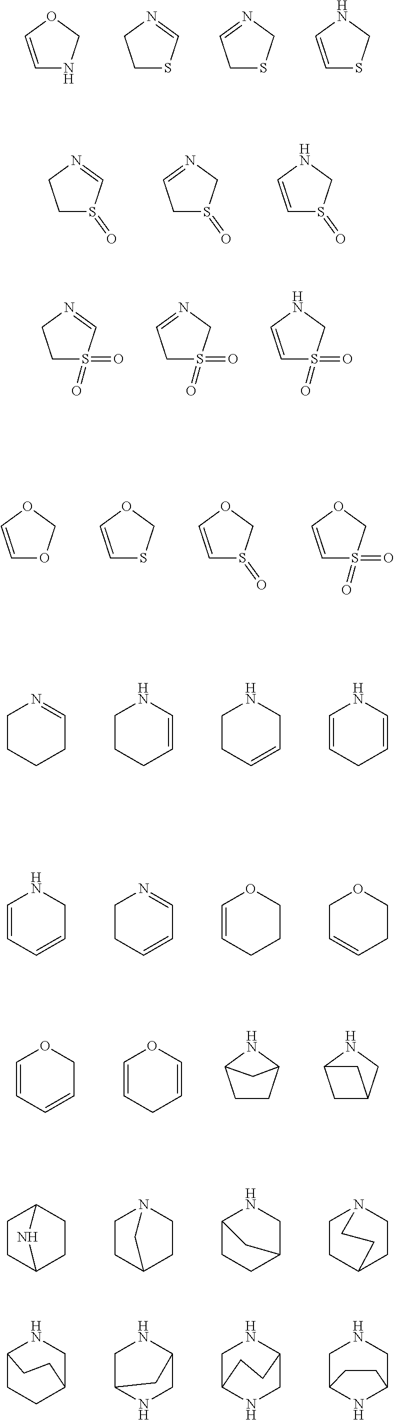

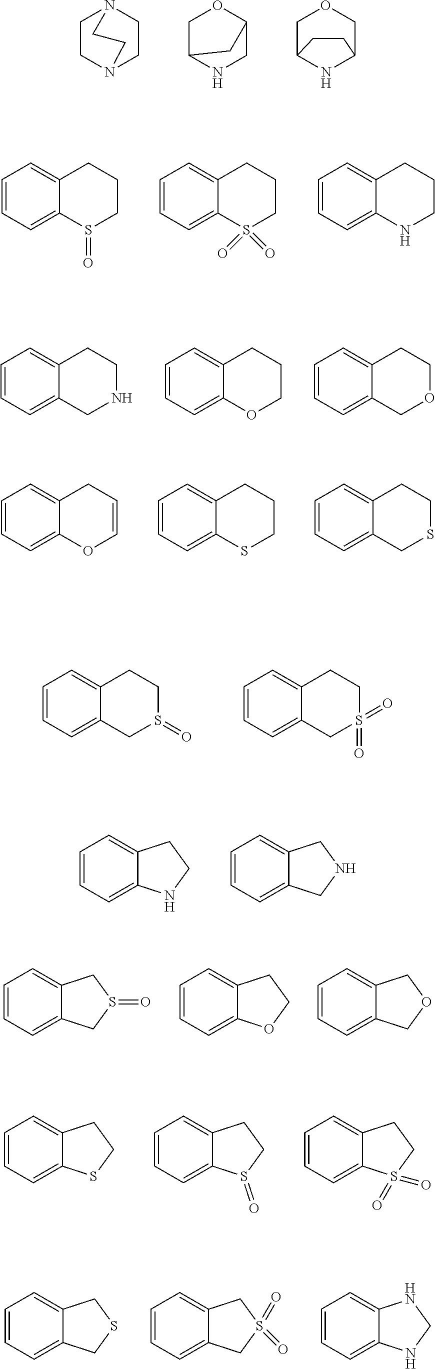

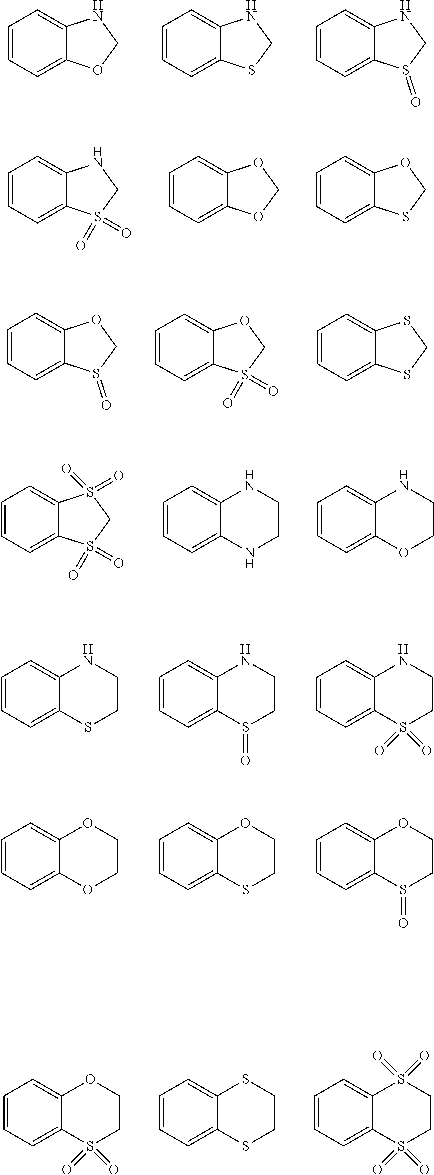

Preferred monocyclic heterocyclyl is 4 to 7 membered and has one or two heteroatoms independently selected from oxygen, nitrogen and sulfur.

Preferred monocyclic heterocyclyls are: piperazinyl, piperidinyl, morpholinyl, pyrrolidinyl, and azetidinyl.

Preferred bicyclic heterocyclyl is 6 to 10 membered and has one or two heteroatoms independently selected from oxygen, nitrogen and sulfur.

Preferred tricyclic heterocyclyl is 9 membered and has one or two heteroatoms independently selected from oxygen, nitrogen and sulfur.

Preferred spiro-heterocyclyl is 7 to 11 membered and has one or two heteroatoms independently selected from oxygen, nitrogen and sulfur.

The above definition of heterocyclyl also applies if heterocyclyl is part of another (combined) group as for example in heterocyclylamino, heterocyclyloxy or heterocyclylalkyl.

If the free valency of a heterocyclyl is saturated, then a heterocycle is obtained.

The term heterocyclylene is also derived from the previously defined heterocyclyl. Heterocyclylene, unlike heterocyclyl, is bivalent and requires two binding partners. Formally, the second valency is obtained by removing a hydrogen atom from a heterocyclyl. Corresponding groups are for example:

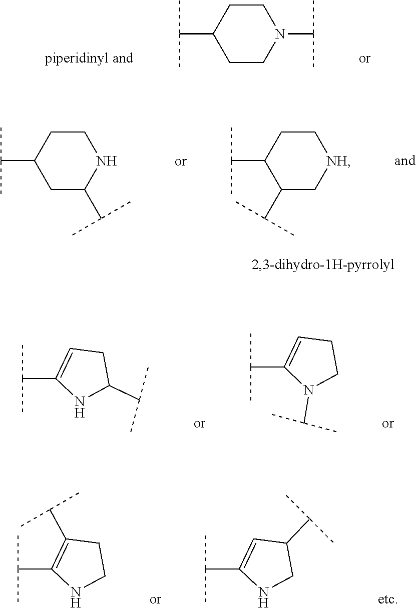

The above definition of heterocyclylene also applies if heterocyclylene is part of another (combined) group as for example in HO-heterocyclyleneamino or H₂N-heterocyclyleneoxy. Heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. The prerequisite for the presence of heteroaryl is a heteroatom and a heteroaromatic system.

If a heteroaryl is to be substituted, the substitutions may take place independently of one another, in the form of mono- or polysubstitutions in each case, on all the hydrogen-carrying carbon and/or nitrogen atoms. Heteroaryl itself may be linked as a substituent to the molecule via every suitable position of the ring system, both carbon and nitrogen. Substituents on heteroaryl do not count for the number of members of a heteroaryl.

Examples of heteroaryl are furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, pyridyl-N-oxide, pyrrolyl-N-oxide, pyrimidinyl-N-oxide, pyridazinyl-N-oxide, pyrazinyl-N-oxide, imidazolyl-N-oxide, isoxazolyl-N-oxide, oxazolyl-N-oxide, thiazolyl-N-oxide, oxadiazolyl-N-oxide, thiadiazolyl-N-oxide, triazolyl-N-oxide, tetrazolyl-N-oxide, indolyl, isoindolyl, benzofuryl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl, benzotriazinyl, indolizinyl, oxazolopyridyl, imidazopyridyl, naphthyridinyl, benzoxazolyl, pyridopyridyl, pyrimidopyridyl, purinyl, pteridinyl, benzothiazolyl, imidazopyridyl, imidazothiazolyl, quinolinyl-N-oxide, indolyl-N-oxide, isoquinolyl-N-oxide, quinazolinyl-N-oxide, quinoxalinyl-N-oxide, phthalazinyl-N-oxide, indolizinyl-N-oxide, indazolyl-N-oxide, benzothiazolyl-N-oxide, benzimidazolyl-N-oxide etc. Further examples are the structures illustrated below, which may be attached via each hydrogen-carrying atom (exchanged for hydrogen):

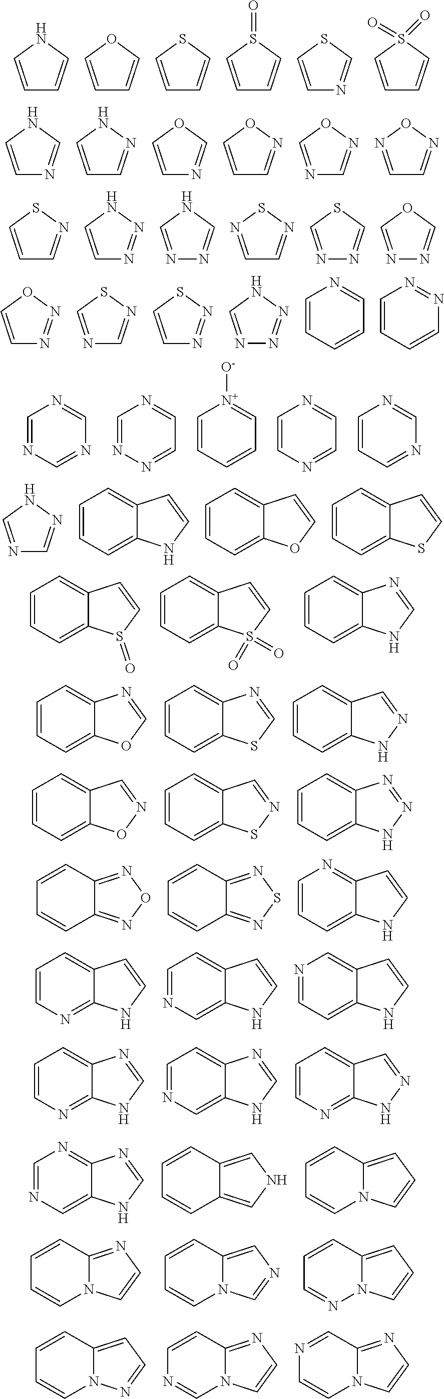

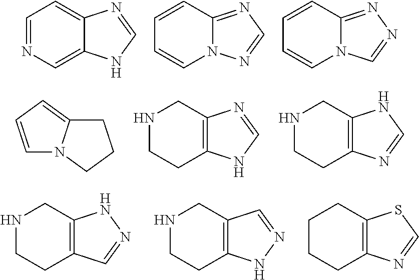

Preferably, heteroaryls are 5-6 membered monocyclic or 9-10 membered bicyclic, each with 1 to 4 heteroatoms independently selected from oxygen, nitrogen and sulfur.

The above definition of heteroaryl also applies if heteroaryl is part of another (combined) group as for example in heteroarylamino, heteroaryloxy or heteroarylalkyl.

If the free valency of a heteroaryl is saturated, a heteroaromatic group is obtained.

The term heteroarylene is also derived from the previously defined heteroaryl. Heteroarylene, unlike heteroaryl, is bivalent and requires two binding partners. Formally, the second valency is obtained by removing a hydrogen atom from a heteroaryl. Corresponding groups are for example:

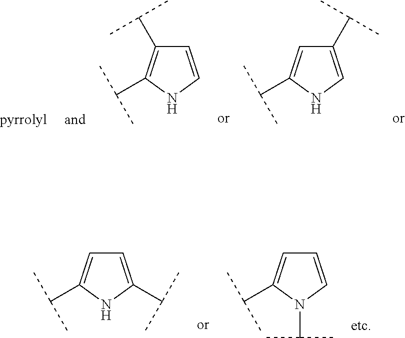

The above definition of heteroarylene also applies if heteroarylene is part of another (combined) group as for example in HO-heteroaryleneamino or H₂N-heteroaryleneoxy.

By substituted is meant that a hydrogen atom which is bound directly to the atom under consideration, is replaced by another atom or another group of atoms (substituent).

Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents on carbon atoms, whereas the bivalent substituents ═O and ═NR may also be a substituent on sulphur. Generally, substitution may be carried out by a bivalent substituent only at ring systems and requires replacement of two geminal hydrogen atoms, i.e. hydrogen atoms that are bound to the same carbon atom that is saturated prior to the substitution. Substitution by a bivalent substituent is therefore only possible at the group —CH₂— or sulphur atoms (═O group or ═NR group only, one or two ═O groups possible or, e.g., one ═O group and one ═NR group, each group replacing a free electron pair) of a ring system.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents. Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

In a representation such as for example

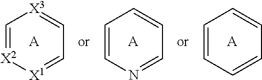

the letter A has the function of a ring designation in order to make it easier, for example, to indicate the attachment of the ring in question to other rings.

For bivalent groups in which it is crucial to determine which adjacent groups they bind and with which valency, the corresponding binding partners are indicated in brackets where necessary for clarification purposes, as in the following representations:

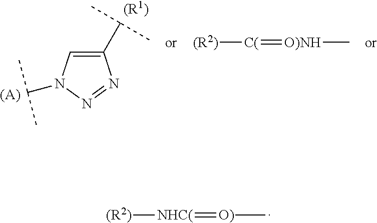

If such a clarification is missing then the bivalent group can bind in both directions, i.e., e.g., —C(═O)NH— also includes —NHC(═O)— (and vice versa).

Groups or substituents are frequently selected from among a number of alternative groups/substituents with a corresponding group designation (e.g. R^{a}, R^{b} etc). If such a group is used repeatedly to define a compound according to the invention in different parts of the molecule, it is pointed out that the various uses are to be regarded as totally independent of one another.

By a therapeutically effective amount for the purposes of this invention is meant a quantity of substance that is capable of obviating symptoms of illness or of preventing or alleviating these symptoms, or which prolong the survival of a treated patient.

### LIST OF ABBREVIATIONS

| | |
|---|---|
| Ac | acetyl |
| ACN | acetonitrile |
| aq. | aqueous |
| ATP | adenosine triphosphate |
| Bn | benzyl |
| Boc | tert-butyloxycarbonyl |
| Bu | butyl |
| c | concentration |
| CDI | 1,1′-carbonyldiimidazole |
| d | day(s) |
| TLC | thin layer chromatography |
| Davephos | 2-dimethylamino-2′-dicyclohexylaminophosphinobiphenyl |
| DBU | 1,8-Diazabicyclo(5.4.0)undec-7-ene |
| DCE | dichloroethane |
| DCM | dichloromethane |
| DEA | diethyl amine |
| DIPEA | N-ethyl-N,N-diisopropylamine (Hünig's base) |
| DMA | dimethylacetamide |
| DMAP | 4-N,N-dimethylaminopyridine |
| DME | 1,2-dimethoxyethane |
| DMF | N,N-dimethylformamide |
| DMSO | dimethylsulphoxide |
| DPPA | diphenylphosphorylazide |
| dppf | 1.1′-bis(diphenylphosphino)ferrocene |
| EDTA | ethylenediaminetetraacetic acid |
| EGTA | ethyleneglycoltetraacetic acid |
| equiv. or eq. | equivalent(s) |
| ESI | electron spray ionization |
| Et | ethyl |
| Et₂O | diethyl ether |
| EtOAc | ethyl acetate |
| EtOH | ethanol |
| h | hour(s) |
| HATU | O-(7-azabenzotriazol-1-yl)-N,N,N′,N′-tetramethyl-uronium |
| | hexafluorophosphate |
| HOBT | 1-hydroxybenzotriazole hydrate |
| HPLC | high performance liquid chromatography |
| i | iso |
| conc. | concentrated |
| LC | liquid chromatography |
| LiHMDS | lithium bis(trimethylsilyl)amide |
| sln. | solution |
| Me | methyl |
| MeOH | methanol |
| min | minute(s) |
| MPLC | medium pressure liquid chromatography |
| MS | mass spectrometry |
| MTBE | methyl tert-butyl ether |
| NMM | N-methylmorpholine |
| NMP | N-methylpyrrolidone |
| NP | normal phase |
| n.a. | not available |
| PBS | phosphate-buffered saline |
| Ph | phenyl |
| Pr | propyl |
| PTSA | p-toluenesulfonic acid |
| Py | pyridine |
| rac | racemic |
| red. | reduction |
| Rf (R_{f}) | retention factor |
| RP | reversed phase |
| RRLC | Rapid resolution liquid chromatography |
| rt | ambient temperature |
| SFC | supercritical fluid chromatography |
| S_{N} | nucleophilic substitution |
| TBAF | tetrabutylammonium fluoride |
| TBDMS | tert-butyldimethylsilyl |
| TBME | tert-butylmethylether |
| TBTU | O-(benzotriazol-1-yl)-N,N,N′,N′-tetramethyl-uronium |
| | tetrafluoroborate |
| tBu | tert-butyl |
| TEA | triethyl amine |
| temp. | temperature |
| tert | tertiary |
| Tf | triflate |
| TFA | trifluoroacetic acid |
| THF | tetrahydrofuran |
| t_{Ret.} | retention time (HPLC) |
| TRIS | tris(hydroxymethyl)-aminomethane |
| TsOH | p-toluenesulphonic acid |
| UPLC | ultra performance liquid chromatography |
| UV | ultraviolet |
| wt | weight |

### EXAMPLES

Features and advantages of the present invention will become apparent from the following detailed examples which illustrate the principles of the invention by way of example without restricting its scope:

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

Microwave reactions are carried out in an initiator/reactor made by Biotage or in an Explorer made by CEM or in Synthos 3000 or Monowave 3000 made by Anton Paar in sealed containers (preferably 2, 5 or 20 mL), preferably with stirring.

### Chromatography

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out on Agilent or Gilson systems with columns made by Waters (names: SunFire™ Prep C18, OBD™ 10 μm, 50×150 mm or SunFire™ Prep 018 OBD™ 5 μm, 30×50 mm or XBridge™ Prep C18, OBD™ 10 μm, 50×150 mm or XBridge™ Prep C18, OBD™ 5 μm, 30×150 mm or XBridge™ Prep C18, OBD™ 5 μm, 30×50 mm) and YMC (names: Actus-Triart Prep C18, 5 μm, 30×50 mm).

Different gradients of H₂O/acetonitrile are used to elute the compounds, while for Agilent systems 5% acidic modifier (20 mL HCOOH to 1 L H₂O/acetonitrile (1/1)) is added to the water (acidic conditions). For Gilson systems the water is added 0.1% HCOOH.

For the chromatography under basic conditions for Agilent systems H₂O/acetonitrile gradients are used as well, while the water is made alkaline by addition of 5% basic modifier (50 g NH₄HCO₃+50 mL NH₃ (25% in H₂O) to 1 L with H₂O). For Gilson systems the water is made alkaline as follows: 5 mL NH₄HCO₃ solution (158 g in 1 L H₂O) and 2 mL NH₃ (28% in H₂O) are replenished to 1 L with H₂O.

The supercritical fluid chromatography (SFC) of the intermediates and example compounds according to the invention is carried out on a JASCO SFC-system with the following columns: Chiralcel OJ (250×20 mm, 5 μm), Chiralpak AD (250×20 mm, 5 μm), Chiralpak AS (250×20 mm, 5 μm), Chiralpak IC (250×20 mm, 5 μm), Chiralpak IA (250×20 mm, 5 μm), Chiralcel OJ (250×20 mm, 5 μm), Chiralcel OD (250×20 mm, 5 μm), Phenomenex Lux C2 (250×20 mm, 5 μm).

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm). The analytical equipment is also equipped with a mass detector in each case.

### HPLC-Mass Spectroscopy/UV-Spectrometry

The retention times/MS-ESI⁺ for characterizing the example compounds according to the invention are produced using an HPLC-MS apparatus (high performance liquid chromatography with mass detector). Compounds that elute at the injection peak are given the retention time t_{Ret.}=0.00.

| Method A | | |
|---|---|---|
| HPLC | Agilent 1100 system | |
| MS | 1200Series LC/MSD(API-ES +/− 3000 V, Quadrupol, G6140) | |
| MSD signal settings | Scan pos/neg 120-900 m/z | |
| Detection signal | 315 nm (bandwidth 170 nm, reference off) | |
| Spectrum range | 230-400 nm | |
| Peak width | <0.01 min | |
| Column | Waters, Xbridge C18, 2.5 μm, 2.1 × 20 mm column | |
| Column temperature | 60° C. | |
| Solvent | A: 20 mM NH₄HCO₃/NH₃ in H₂O pH 9 | |
| | B: ACN HPLC grade | |
| Flow | 1.00 mL/min | |
| Gradient | 0.00-1.50 min | 10% to 95% B |
| | 1.50-2.00 min | 95% B |
| | 2.00-2.10 min | 95% to 10% B |
| Method B | | |
| HPLC | Agilent 1260 system | |
| MS | 1200 Series LC/MSD (MM-ES + APCI +/− 3000 V, Quadrupol, | |
| | G6130) | |
| Detection | UV: 254 nm (bandwidth 8, reference off) | |
| | UV: 230 nm (bandwidth 8, reference off) | |
| | UV spectrum range: 190-400 nm; step: 4 nm | |
| | MS: positive and negative mode | |
| Mass range | 100-800 m/z | |
| Column | Waters; Part. No. 186003389; XBridge BEH C18, | |
| | 2.5 μm, 30 × 2.1 mm | |
| Column temperature | 45° C. | |
| Solvent | A: 5 mM NH₄HCO₃/19 mM NH₃ in H₂O; B: ACN (HPLC grade) | |
| Flow | 1.40 mL/min | |
| Gradient | 0.00-1.00 min: 5% B to 100% B | |
| | 1.00-1.37 min: 100% B | |
| | 1.37-1.40 min: 100% B to 5% B | |
| Method C | | |
| HPLC | Agilent 1260 Series | |
| MS | Agilent LC/MSD Quadrupole | |
| Detection | MS: positive and negative mode | |
| Mass range | 100-750 m/z | |
| Column | Waters X-Bridge BEH C18, 2.5 μm, 2.1 × 30 mm XP | |
| Column temperature | 45° C. | |
| Solvent | A: 20 mM NH₄HCO₃/30 mM NH₃ in H₂O; B: ACN (HPLC grade) | |
| Flow | 1.40 mL/min | |
| Gradient | 0.00-1.00 min: 15% B to 95% B | |
| | 1.00-1.30 min: 95% B | |
| Method D | | |
| HPLC | Agilent 1100/1200 system | |
| MS | 1200 Series LC/MSD (MM-ES + APCI +/− 3000 V, Quadrupol, | |
| | G6130B) | |
| MSD signal settings | Scan pos 150-750 | |
| Detection signal | UV 254 nm, 230 nm, 214 nm (bandwidth 8, reference off) | |
| Spectrum | range: 190-400 nm; slit: 4 nm | |
| Peak width | >0.0031 min (0.063 s response time, 80 Hz) | |
| Column | Waters, Part. No. 186003389, XBridge BEH C18, 2.5 μm, 2.1 × | |
| | 30 mm) column | |
| Column temperature | 45° C. | |
| Solvent | A: 5 mM NH₄HCO₃/18 mM NH₃ in H2O (pH = 9.2) | |
| | B: ACN (HPLC grade) | |
| Flow | 1.4 mL/min | |
| Gradient | 0.0-1.0 min | 15% to 95% B |
| | 1.0-1.1 min | 95% B |
| | Stop time: 1.3 min | |
| Method E | | |
| HPLC | Agilent 1100/1200 system | |
| MS | 1200 Series LC/MSD (MM-ES + APCI +/− 3000 V, Quadrupol, | |
| | G6130B) | |
| MSD signal settings | Scan pos/neg 150-750 | |
| Detection signal | UV 254 nm, 230 nm, 214 nm (bandwidth 8, reference off) | |
| Spectrum | range: 190-400 nm; slit: 4 nm | |
| Peak width | >0.0031 min (0.063 s response time, 80 Hz) | |
| Column | Waters, Part. No. 186003389, XBridge BEH C18, 2.5 μm, 2.1 × | |
| | 30 mm) column | |
| Column temperature | 45° C. | |
| Solvent | A: 5 mM NH₄HCO₃/18 mM NH₃ in H2O (pH = 9.2) | |
| | B: ACN (HPLC grade) | |
| Flow | 1.4 mL/min | |
| Gradient | 0.0-1.0 min | 15% to 95% B |
| | 1.0-1.1 min | 95% B |
| | Stop time: 1.3 min | |
| Method F | | |
| HPLC | Agilent 1100/1200 system | |
| MS | 1200 Series LC/MSD (API-ES +/− 3000/3500 V, Quadrupol, G6140A) | |
| MSD signal settings | Scan pos/neg 150-750 | |
| Detection signal | UV 254 nm, 230 nm, 214 nm (bandwidth 10, reference off) | |
| Spectrum | range: 190-400 nm; slit: 4 nm | |
| Peak width | >0.0031 min (0.063 s response time, 80 Hz) | |
| Column | YMC; Part. No. TA12S03-0302WT; Triart C18, 3 μm, 12 nm; | |
| | 30 × 2.0 mm column | |
| Column temperature | 45° C. | |
| Solvent | A: H₂O + 0.11% formic acid | |
| | B: ACN + 0.1% formic acid (HPLC grade) | |
| Flow | 1.4 mL/min | |
| Gradient | 0.0-1.0 min | 15% to 95% B |
| | 1.0-1.1 min | 95% B |
| Stop time: | 1.23 min | |
| Method G | | |
| UPLC-MS | Waters Acquity-UPLC-SQ Detector-2 | |
| MSD signal settings | Scan pos & Neg 100-1500, | |
| | Source Voltage: Capillary Vol(kV)- 3.50, Cone(V): 50 | |
| | Source Temp: Desolvation Temp(° C.): 350 | |
| | Source Gas Flow: Desolvation (L/Hr): 750, Cone(L/Hr): 50 | |
| Detection signal | Diode Array | |
| Spectrum | Range: 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.30 min | 97% B |
| | 0.30-2.20 min | 97% to 2% B |
| | 2.20-3.30 min | 2% B |
| | 3.30-4.50 min | 2% to 97% B |
| | 4.50-4.51 min | 97% B |
| Method H | | |
| UPLC-MS | Waters Acquity-Binary Solvent Manager-UPLC-SQ Detector-2 | |
| MSD signal settings | Scan pos & Neg 100-1500, | |
| | Source Voltage: Capillary Vol(kV)- 3.50, Cone(V): 50 | |
| | Source Temp: Desolvation Temp(° C.): 350 | |
| | Source Gas Flow: Desolvation (L/Hr): 750, Cone(L/Hr): 50 | |
| Detection signal | Diode Array | |
| Spectrum | Range: 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.40 min | 97% B |
| | 0.40-2.50 min | 97% to 2% B |
| | 2.50-3.40 min | 2% B |
| | 3.40-3.50 min | 2% to 97% B |
| | 3.50-4.0 min | 97% B |
| Method I | | |
| LC-MS | Waters Arc-HPLC-SQ Detector-2 | |
| MSD signal settings | ESI Scan pos & neg | |
| | Capillary Voltage 3.50 Kv cone voltage 30 V Desolvation gas | |
| | 750 L/hr Desolvation Temp 350° c. | |
| Column | X-Bridge C18, 4.6 × 75 mm, 3.5μ | |
| Column temperature | 35° C. | |
| Solvent | A: 10 mM Ammonium Acetate in water | |
| | B: ACN | |
| Flow | 1.0 mL/min | |
| Gradient | 0.0-0.75 min | 5% B |
| | 0.75-1.50 min | 5% to 40% B |
| | 1.50-5.0 min | 40% to 98% B |
| | 5.0-7.0 min | 98% B |
| Method J | | |
| LC-MS | Waters Acquity-UPLC-SQ Detector-2 | |
| MSD signal settings | ESI Scan pos & neg | |
| | Capillary Voltage 3.50 Kv cone voltage 50 V Desolvation gas | |
| | 750 L/h Desolvation Temp 350° C. | |
| Column | Waters Acquity-UPLC-SQ Detector-2 | |
| Column temperature | 35° C. | |
| Solvent | A: 0.05% TFA in ACN | |
| | B: 0.05% TFA in water | |
| Flow | 0.6 mL/min | |
| Gradient | 0.0-0.3 min | 97% B |
| | 0.3-2.2 min | 97% to 2% B |
| | 2.2-3.3 min | 2% B |
| Method K | | |
| LC-MS | Waters Arc-HPLC-SQ Detector-2 | |
| MSD signal settings | ESI Scan pos & neg | |
| | Capillary Voltage 3.50 Kv cone voltage 30 V Desolvation gas | |
| | 750 L/hr Desolvation Temp 350° C. | |
| Column | X-Bridge C18, 4.6 × 50 mm, 3.5μ | |
| Column temperature | 35° C. | |
| Solvent | A: 10 mM Ammonium Acetate in water | |
| | B: ACN | |
| Flow | 2.0 mL/min | |
| Gradient | 0.0-0.2 min | 10% B |
| | 0.2-2.50 min | 10% to 75% B |
| | 2.50-3.0 min | 75% to 100% B |
| | 3.0-4.8 min | 100% B |
| Method L | | |
| HPLC | Agilent 1260 Series | |
| MS | Agilent LC/MSD Quadrupole | |
| Detection | MS: positive and negative mode | |
| Mass range | 550-1200 m/z | |
| Column | Waters X-Bridge BEH C18, 2.5 μm, 2.1 × 30 mm XP | |
| Column temperature | 45° C. | |
| Solvent | A: 20 mM NH₄HCO₃/30 mM NH₃ in H₂O; B: ACN (HPLC grade) | |
| Flow | 1.40 mL/min | |
| Gradient | 0.00-1.50 min: 50% B to 95% B | |
| | 1.50-2.00 min: 95% B | |
| Method M | | |
| HPLC | Agilent 1260 Series | |
| MS | Agilent LC/MSD Quadrupole | |
| Detection | MS: positive and negative mode | |
| Mass range | 550-1200 m/z | |
| Column | Waters X-Bridge BEH C18, 2.5 μm, 2.1 × 30 mm XP | |
| Column temperature | 45° C. | |
| Solvent | A: 20 mM NH₄HCO₃/30 mM NH₃ in H₂O; B: ACN (HPLC grade) | |
| Flow | 1.40 mL/min | |
| Gradient | 0.00-1.00 min: 50% B to 95% B | |
| | 1.00-1.30 min: 95% B | |
| Method N | | |
| HPLC | Agilent 1260 Series | |
| MS | Agilent LC/MSD Quadrupole | |
| Detection | MS: positive and negative mode | |
| Mass range | 100-750 m/z | |
| Column | YMC-Triart C18, 3 μm, 12 nm, 2.0 × 30 mm | |
| Column temperature: | 45° C. | |
| Solvent | A: H₂O + 0.11% formic acid; B: ACN (HPLC grade) + 0.1% formic | |
| | acid | |
| Flow: | 1.40 mL/min | |
| Gradient: | 0.00-1.00 min: 15% B to 95% B | |
| | 1.00-1.30 min: 95% B | |
| Method O | | |
| HPLC | Waters - Alliance 2996 | |
| Detection signal | PDA Detector | |
| Spectrum | Range: 200-400 nm; Resolution: 1.2 nm | |
| Sampling rate | 1 point/sec | |
| ELSD Parameters | Gas Pressure: 50 PSI, Drift tube Temp: 50° C., Gain: 500 | |
| Column | Atlantis T3 (4.6 × 250 mm) 5.0 μm | |
| Column temperature | Ambient | |
| Solvent | A: 10 mM ammonium acetate in water | |
| | B: ACN | |
| Flow | 0.7 mL/min | |
| Gradient | 0.0-1.20 min | 2% B |
| | 1.2-10.0 min | 2% to 98% B |
| | 10.0-12.0 min | 98% B |
| | 12.0-14.0 min | 97% to 2% B |
| | 14.0-16.0 min | 2% B |
| Method P | | |
| UPLC-MS | Waters Acquity-UPLC-SQ Detector-2 | |
| MSD signal settings | Scan Positive & Negative 100-1500, | |
| | Source Voltage: Capillary Voltage(kV)- 3.50, Cone(V): 50 | |
| | Source Temp: Desolvation Temp(° C.): 350 | |
| | Source Gas Flow: Desolvation (L/Hr): 650 | |
| Detection signal | Diode Array | |
| Spectrum | Range: 200-400 nm; Resolution: 1.2 nm | |
| Sampling rate | 10 point/sec | |
| ELSD Parameters: | GAS: 2.0 SLM, Nebulizer Temp: 40° C., Evaporative Temp: 45° C. | |
| Column | AQUITY UPLC BEH C18 1.7 μm, 2.1 × 50 mm | |
| Column temperature | 50° C. | |
| Solvent | A: 0.05% formic acid in water | |
| | B: 0.05% formic acid in ACN | |
| Flow | 0.6 mL/min | |
| Gradient | 0.0-2.20 min | 3% to 98% B |
| | 2.20-3.20 min | 98% B |
| | 3.20-3.50 min | 98% to 3% B |
| | 3.50-4.20 min | 2% B |
| Method Q | | |
| HPLC-MS | Waters Arc-HPLC with 2998PDA Detector and SQ Detector-2 | |
| MSD signal settings | Scan Pos & Neg 100-1500, | |
| | Source Votage: Capillary Vol(kV)- 3.50, Cone(V): 30 | |
| | Source Temp: Desolvation Temp(° C.): 350 | |
| | Source Gas Flow: Desolvation (L/h): 750 | |
| Detection signal | PDA Detector | |
| Spectrum | Range: 200-400 nm; Resolution: 1.2 nm | |
| Sampling rate | 10 point/sec | |
| Column | X-Bridge C18, 4.6 × 50 mm, 3.5 μm | |
| Column temperature | 35° C. | |
| Solvent | A: 10 mM ammonium acetate in water | |
| | B: ACN | |
| Flow | 1.0 mL/min | |
| Gradient | 0.0-0.75 min | 5% B |
| | 0.75-1.50 min | 5% to 40% B |
| | 1.50-5.0 min | 40% to 98% B |
| | 5.0-7.0 min | 98% B |
| | 7.0-9.0 min | 98% to 5% B |
| | 9.0-10.01 min | 5% B |
| Method R | | |
| HPLC | Agilent 1200 system | |
| Column | Chiralpak IE, 5.0 μm, 2.1 × 150 mm column | |
| Column temperature | 40° C. | |
| Solvent | EtOH/Heptane 1:1 + 0.1% diethylamine (isocratic) | |
| Flow | 0.60 mL/min | |
| Method X | | |
| UPLC-MS | Waters Aquity H Class LC system coupled with a Waters | |
| | ZQ/3100/SQD2 mass spectrometer | |
| MSD signal settings | Scan pos & Neg 100-1500, | |
| Detection signal | Diode Array | |
| Spectrum | Range: 190-400 nm; Resolution: 1.2 nm | |
| Column | YMC triart C18 column (3 μm, 33 × 2.1 mm) | |
| Column temperature | 30° C. | |
| Solvent | A: 5 mM ammonium acetate in water/0.05% formic acid in water | |
| | B: 5 mM ammonium acetate in acetonitrile:water (90:10)/ | |
| | 0.05% formic acid in water | |
| Flow | 1 mL/min | |
| Gradient | 0.0-0.75 min | 2% B |
| | 0.75-1.00 min | 2% to 10% B |
| | 1.00-2.00 min | 10% to 98% B |
| | 2.00-2.50 min | 98% B |
| | 2.50-2.90 min | 98% to 2% B |
| | 2.90-3.00 min | 2% B |
| Method Y | | |
| HPLC | Agilent 1100 system | |
| MS | 1200Series LC/MSD(API-ES +/− 3000 V, Quadrupol, G6140) | |
| MSD signal settings | Scan pos/neg 120-1500 m/z | |
| Detection signal | 315 nm (bandwidth 170 nm, reference off) | |
| Spectrum range | 230-400 nm | |
| Peak width | <0.01 min | |
| Column | Waters, Xbridge C18, 2.5 μm, 2.1 × 20 mm column | |
| Column temperature | 60° C. | |
| Solvent | A: 20 mM aq. NH₄HCO₃/NH3 pH 9 | |
| | B: ACN HPLC grade | |
| Flow | 1.00 mL/min | |
| Gradient | 0.00-1.50 min | 10% to 95% B |
| | 1.50-2.00 min | 95% B |
| | 2.00-2.10 min | 95% to 10% B |
| Method Z | | |
| UPLC-MS | Waters Acquity-Binary Solvent Manager-UPLC-SQ Detector-2 | |
| MSD signal settings | Scan pos & Neg 100-1500, | |
| | Source Voltage: Capillary Vol(kV)- 3.50, Cone(V): 50 | |
| | Source Temp: Desolvation Temp(° C.): 350 | |
| | Source Gas Flow: Desolvation(L/Hr): 700, Cone(L/Hr): 50 | |
| Detection signal | Diode Array | |
| Spectrum Range: | 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.40 min | 97% B |
| | 0.40-2.50 min | 97% to 2% B |
| | 2.50-3.40 min | 2% B |
| | 3.40-3.50 min | 2% to 97% B |
| | 3.50-4.0 min | 97% B |

| | |
|---|---|
| GCMS | |
| Method U | |
| GC | Agilent Technologies-7890B GC System with 7693 |
| | Auto Sampler and 5977A MSD |
| Injection Temperature | 230° C. |
| Column Flow | 2.0 mL/min |
| Solvent delay | 1.5 min |
| Split Ratio | 10:01 |
| Column Oven Temperature Program | 100° C./1 min, 20° C./min/310°/5 min |
| Total run time | 16 min |
| Interface Temperature | 150° C. |
| lon Source Temperature | 230° C. |
| Gas | He |
| Column & Column dimension | ZB-5MS (30 m × 0.32 mm; 1 μm) |
| MSD Scan Range | 50-900 |
| Method V | |
| GC | Agilent Technologies-7890B GC System with 7693 |
| | Auto Sampler and 5977A MSD |
| Injection Temperature | 230° C. |
| Column Flow | 2.0 mL/min |
| Solvent delay | 1.5 min |
| Split Ratio | 10:01 |
| Column Oven Temperature Program | 40° C./2 min, 15° C./min/200°/1 min, |
| | 25° C./min/310°/0 min, |
| Total run time | 18 min |
| Interface Temperature | 150° C. |
| lon Source Temperature | 230° C. |
| Gas | He |
| Column & Column dimension | ZB-5MS (30 m × 0.32 mm; 1 μm) |
| MSD Scan Range | 50-900 |
| Method W | |
| GC | Agilent Technologies-7890B GC System with 7693 |
| | Auto Sampler and 5977A MSD |
| Injection Temperature | 230° C. |
| Column Flow | 2.0 mL/min |
| Solvent delay | 1.5 min |
| Split Ratio | 10:01 |
| Column Oven Temperature Program | 60° C./3 min, 20° C./min/310°/2 min |
| Total run time | 18 min |
| Interface Temperature | 150° C. |
| Ion Source Temperature | 230° C. |
| Gas | He |
| Column & Column dimension | ZB-5MS (30 m × 0.32 mm; 1 μm) |
| Method SFC-1 | |
| Make | Waters UPC²-MS |
| Soft | Empower3 |
| MS | QDa |
| Column | CHIRALCEL OX-3(4.6*150 MM) 3 μm |
| A-Solvent | CO2 |
| B-solvent | ACN |
| Total Flow | 3 g/min |
| % of Co-Solvent | 15 |
| ABPR | 1500 psi |
| Colum temp | 30° C. |
| PDA range | 200 nm to 400 nm |
| Resolution | 1.2 nm |
| MS Parameters | — |
| QDa MS scan range | 100 Da to 1000 Da |
| | Cone voltage |
| Positive scan | 20 V |
| Negative Scan | 15 V |

The compounds according to the invention and intermediates are prepared by the methods of synthesis described hereinafter in which the substituents of the general formulae have the meanings given hereinbefore. These methods are intended as an illustration of the invention without restricting its subject matter and the scope of the compounds claimed to these examples. Where the preparation of starting compounds is not described, they are commercially obtainable or their synthesis is described in the prior art or they may be prepared analogously to known prior art compounds or methods described herein, i.e. it is within the skills of an organic chemist to synthesize these compounds. Substances described in the literature can be prepared according to the published methods of synthesis. If a chemical structure in the following is depicted without exact configuration of a stereo center, e.g. of an asymmetrically substituted carbon atom, then both configurations shall be deemed to be included and disclosed in such a representation. The representation of a stereo center in racemic form shall always deem to include and disclose both enantiomers (if no other defined stereo center(s) exists) or all other potential diastereomers and enantiomers (if additional, defined or undefined, stereo centers exist).

For example, certain compounds of the invention can be made according to the procedure described in Scheme 1 below.

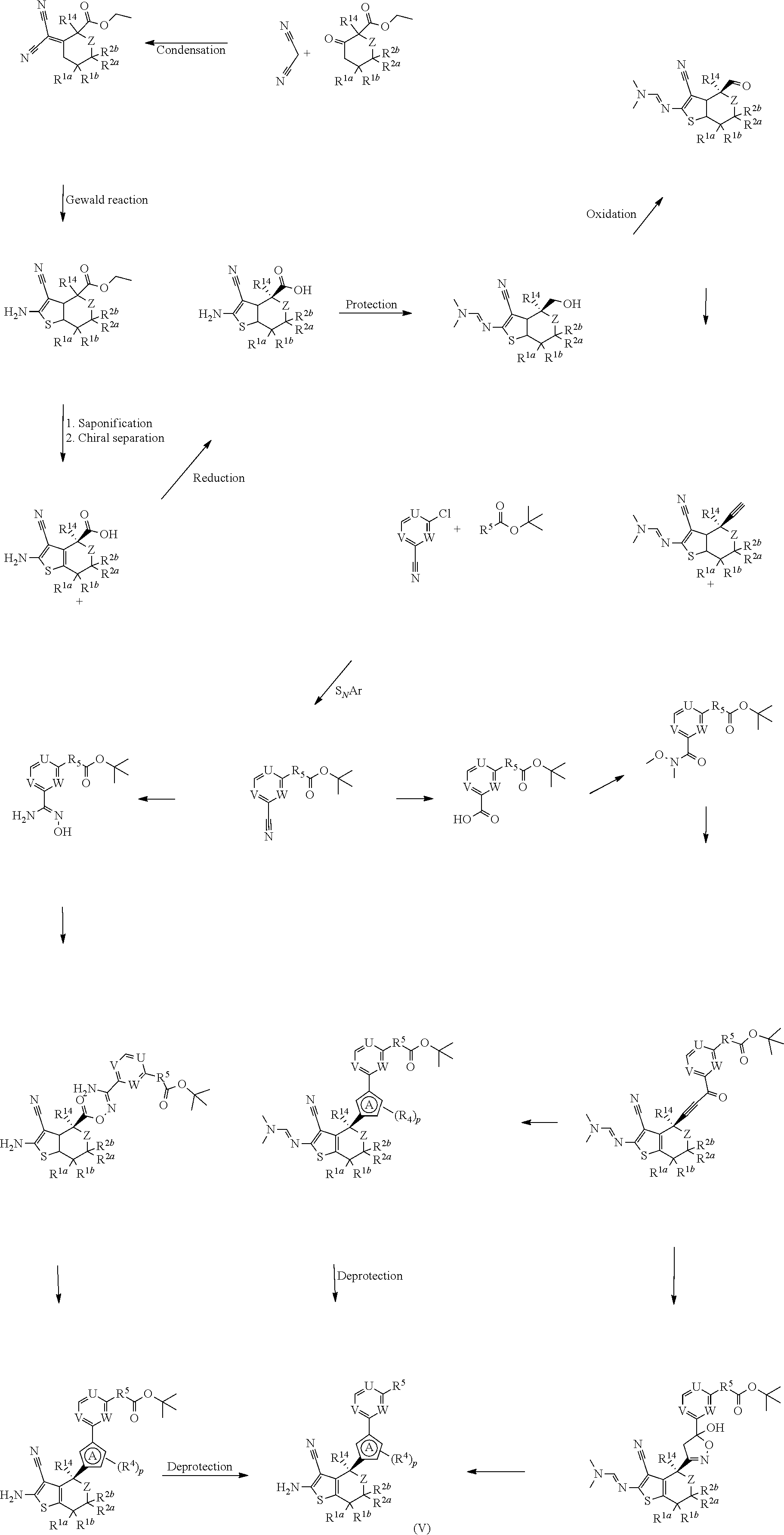

### Synthesis of Various Building Blocks

### Experimental Procedure for the Synthesis of 1-benzyl 4-tert-butyl (2S)-2-methyl-1,4-diazepane-1,4-dicarboxylate

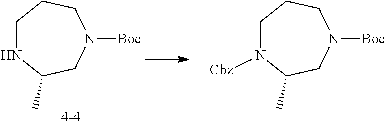 [SMILES: C[C@H]1CN(C(=O)OC(C)(C)C)CCCN1 ; C[C@H]1CN(C(=O)OC(C)(C)C)CCCN1C(=O)OCc1ccccc1]

Tert-butyl (3S)-3-methyl-1,4-diazepane-1-carboxylate (500 mg, 2.33 mmol) is dissolved in dry THF (5.00 mL) together with triethylamine (485 μL, 3.5 mmol, 1.5 eq.) and the mixture is cooled to 0° C. Benzyl chloroformate (519 μL, 3.5 mmol, 1.5 eq.) is added portionwise and the mixture is stirred for 2 h and allowed to reach rt overnight. After complete conversion water is added to the mixture and the product is extracted with DCM and the combined extracts are dried, filtered and concentrated. The crude product (1-benzyl 4-tert-butyl (2S)-2-methyl-1,4-diazepane-1,4-dicarboxylate) is used for the next step without further purification. (HPLC method B, tᵣₑₜ=0.766 min, [M+H]⁺=249/293).

### Experimental Procedure for the Synthesis of benzyl (2S)-2-methyl-1,4-diazepane-1-carboxylate

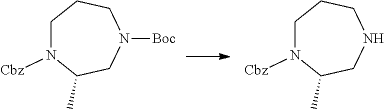 [SMILES: C[C@H]1CN(C(=O)OC(C)(C)C)CCCN1C(=O)OCc1ccccc1 ; C[C@H]1CNCCCN1C(=O)OCc1ccccc1]

1-benzyl 4-tert-butyl (2S)-2-methyl-1,4-diazepane-1,4-dicarboxylate (813 mg, 2.33 mmol) is dissolved in DCM (25.00 mL) and treated with HCl (4 M in dioxane, 11.67 mL, 46.66 mmol, 20.0 eq.). The mixture is stirred for 2 h at rt. After complete conversion the mixture is concentrated under reduced pressure and the product is isolated via basic reversed phase chromatography (gradient elution: 10% to 70% acetonitrile in water). (HPLC method B, tᵣₑₜ=0.478 min, [M+H]⁺=249).

Experimental Procedure for the Synthesis of benzyl (2S)-2,4-dimethyl-1,4-diazepane-1-carboxylate

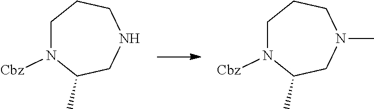 [SMILES: C[C@H]1CNCCCN1C(=O)OCc1ccccc1 ; C[C@H]1CN(C)CCCN1C(=O)OCc1ccccc1]

Benzyl (2S)-2-methyl-1,4-diazepane-1-carboxylate (4.0 g, 16.12 mmol) is dissolved in dry DCM (50.00 mL) and treated with formaldehyde (37% in water, 1.21 mL, 16.12 mmol, 1.00 eq.) and acetic acid (92 μL, 1.61 mmol, 0.10 eq.). The mixture is stirred for 15 min and then sodium triacetoxyborohydride (6.335 g, 29.00 mmol, 1.80 eq.) is added and the mixture is stirred for 1 h at rt. After complete conversion water is added to the mixture and the product is extracted with DCM and the combined extracts are dried, filtered and concentrated. The crude product (benzyl (2S)-2,4-dimethyl-1,4-diazepane-1-carboxylate) is purified via normal phase chromatography (DCM/MeOH).

Experimental Procedure for the Synthesis of benzyl (2S)-4-(2-methoxyethyl)-2-methyl-1,4-diazepane-1-carboxylate

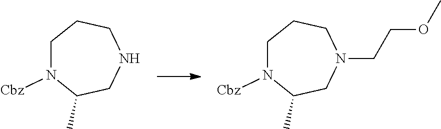 [SMILES: C[C@H]1CNCCCN1C(=O)OCc1ccccc1 ; COCCN1CCCN(C(=O)OCc2ccccc2)[C@@H](C)C1]

To a stirred solution of benzyl (2S)-2-methyl-1,4-diazepane-1-carboxylate (250.0 mg, 1.00 mmol) in dry DMF (5.00 mL), K₂CO₃ (0.303 g, 2.51 mmol, 2.50 eq.) is added followed by 1-bromo-2-methoxy-ethane (0.122 g, 1.00 mmol, 1.00 eq.). The reaction mixture is stirred at 80° C. for 16 h. After complete conversion water is added to the mixture and the product is extracted with EtOAc and the combined extracts are dried, filtered, and concentrated. The crude product is purified via normal phase chromatography (DCM/MeOH).

### Experimental Procedure for the Synthesis of tert-butyl (2S)-4-(3-methoxypropyl)-2-methyl-1,4-diazepane-1-carboxylate

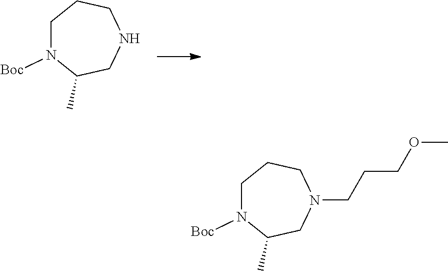 [SMILES: C[C@H]1CNCCCN1C(=O)OC(C)(C)C ; COCCCN1CCCN(C(=O)OC(C)(C)C)[C@@H](C)C1]

To a stirred solution of (S)-tert-butyl 2-methyl-1,4-diazepane-1-carboxylate (500.0 mg, 2.33 mmol) in ACN is added K₂CO₃ (1.612 g, 11.66 mmol, 5.0 eq.) followed by 1-bromo-3-methoxy-propane (535.5 mg, 3.50 mmol, 1.5 eq.). The reaction mixture is stirred at 45° C. for 16 h. After complete conversion water is added to the mixture and the product is extracted with EtOAc and the combined extracts are dried, filtered, and concentrated. The crude product is purified via normal phase chromatography (DCM/MeOH 0-10% gradient).

### Experimental Procedure for the Synthesis of (3S)-1-(3-methoxypropyl)-3-methyl-1,4-diazepane

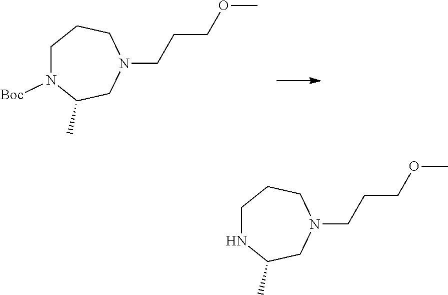 [SMILES: COCCCN1CCCN(C(=O)OC(C)(C)C)[C@@H](C)C1 ; COCCCN1CCCN[C@@H](C)C1]

To a stirred solution of tert-butyl (2S)-4-(3-methoxypropyl)-2-methyl-1,4-diazepane-1-carboxylate (100.0 mg, 0.35 mmol) in MeOH is added conc. HCl (0.595 mg, 5.875 mmol). The reaction mixture is stirred at room temperature for 2 h. After complete conversion the solvent is evaporated under reduced pressure and the crude product is directly used without further purification.

### Experimental Procedure for the Synthesis of (3S)-1,3-dimethyl-1,4-diazepane

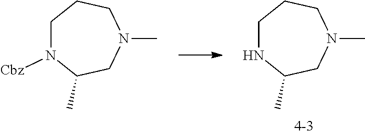

Benzyl (2S)-2,4-dimethyl-1,4-diazepane-1-carboxylate (3.00 g, 11.44 mmol) is dissolved in MeOH (20.0 mL) and palladium (10% on carbon, 360 mg) is added. The mixture is stirred in a hydrogenation reactor under 5 bar of hydrogen pressure for 16 h at rt. After complete conversion the catalyst is filtered off and the residue is concentrated. The crude product is used for the following step without purification.

Additional intermediates are available in an analogous manner.

### Experimental Procedure for the Synthesis of K-20a

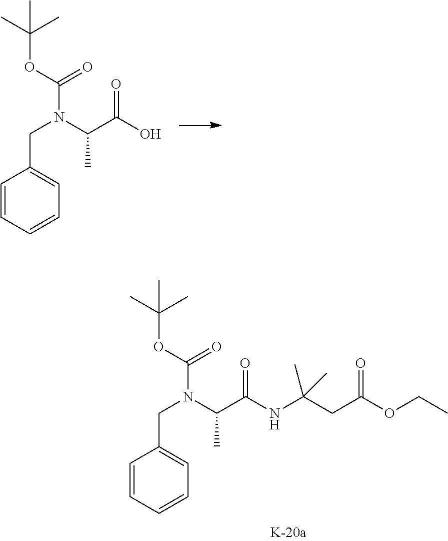 [SMILES: C[C@@H](C(=O)O)N(Cc1ccccc1)C(=O)OC(C)(C)C ; CCOC(=O)CC(C)(C)NC(=O)[C@H](C)N(Cc1ccccc1)C(=O)OC(C)(C)C]

To a stirred solution of (2S)-2-{benzyl[(tert-butoxy)carbonyl]amino}propanoic acid (24.00 g, 85.92 mmol, 1.0 eq.) in DCM (200 mL) is added 1-ethyl-3-(3-dimethylaminopropyl) carbodiimide hydrochloride (24.71 g, 128.88 mmol, 1.5 eq.), 1-hydroxy benzo triazole (19.74 g, 128.88 mmol, 1.5 eq.), and NEt₃ (47.81 mL, 343.67 mmol, 4.0 eq.). The reaction mixture is stirred at rt for 5 min. 3-amino-3-methyl-butyric acid ethyl ester hydrochloride (23.41 g, 128.88 mmol, 1.5 eq.) is added and the reaction mixture is stirred at rt for 16 h. After complete conversion the reaction mixture is diluted with DCM and washed with water. The organic layer is washed with brine, dried over Na₂SO₄ and concentrated under reduced pressure. The crude product is purified by column chromatography to obtain K-20a.

TABLE 1

| | | tᵣₑₜ | | |
|---|---|---|---|---|
| # | structure | [min] | [M + H]⁺ | HPLC method |
| K-20a | 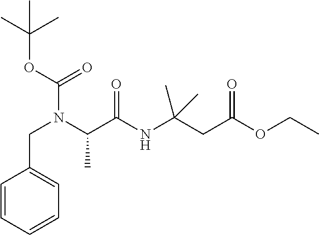 [SMILES: CCOC(=O)CC(C)(C)NC(=O)[C@H](C)N(Cc1ccccc1)C(=O)OC(C)(C)C] | 1.99 | 407 | X |

### Experimental Procedure for the Synthesis of K-21a

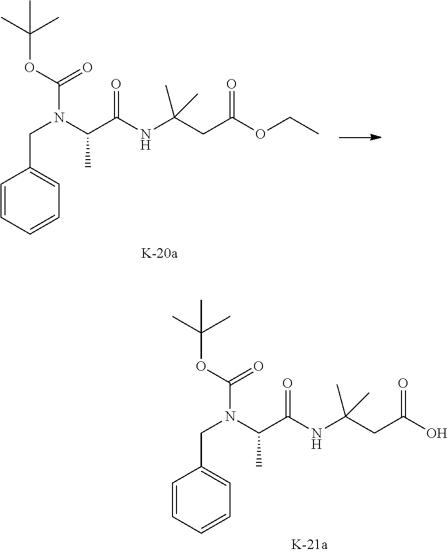

To a stirred solution of K-20a (16.00 g, 39.36 mmol, 1.0 eq.) in THF (40 mL) and water (20 mL) is added lithium hydroxide (2.48 g, 59.04 mmol, 1.5 eq.) and the reaction mixture is stirred at rt for 16 h. After complete conversion the reaction mixture is diluted with water and the organic layer is separated. The aqueous layer is washed with EtOAc. The aqueous layer is acidified with citric acid and extracted with EtOAc. The combined organic layer is dried over Na₂SO₄ and concentrated under reduced pressure to obtain the crude product. The crude product K-21a is used in the next step without further purification.

TABLE 2

| | | tᵣₑₜ | | |
|---|---|---|---|---|
| # | structure | [min] | [M + H]⁺ | HPLC method |
| K-21a | 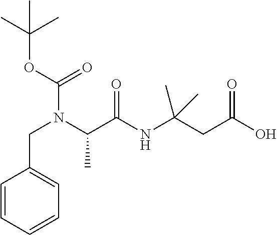 [SMILES: C[C@@H](C(=O)NC(C)(C)CC(=O)O)N(Cc1ccccc1)C(=O)OC(C)(C)C] | 1.60 | 279 | X |

### Experimental Procedure for the Synthesis of K-22a

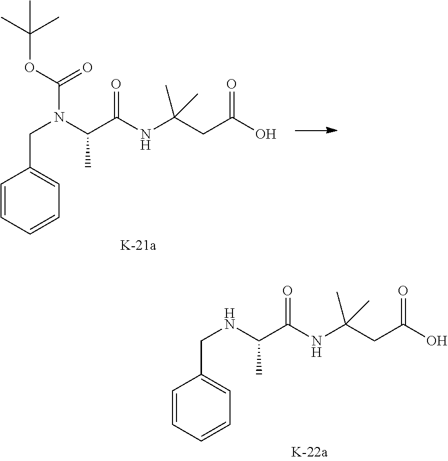 [SMILES: C[C@@H](C(=O)NC(C)(C)CC(=O)O)N(Cc1ccccc1)C(=O)OC(C)(C)C ; C[C@H](NCc1ccccc1)C(=O)NC(C)(C)CC(=O)O]

To a stirred solution of intermediate K-21a (20.00 g, 52.84 mmol, 1.0 eq.) in DCM (100 mL) is added HCl (200 mL, 4 N in 1,4 dioxane) and the reaction mixture is stirred at rt for 4 h. After complete conversion the volatiles are removed under reduced pressure to get the crude product. The crude product is triturated with pentane to afford intermediate K-22a which is used in next step without any other purification.

TABLE 3

| | | tᵣₑₜ | | |
|---|---|---|---|---|
| # | structure | [min] | [M + H]⁺ | HPLC method |
| K-22a | 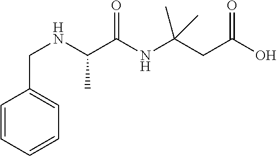 [SMILES: C[C@H](NCc1ccccc1)C(=O)NC(C)(C)CC(=O)O] | 1.30 | 279 | X |

### Experimental Procedure for the Synthesis of K-23a

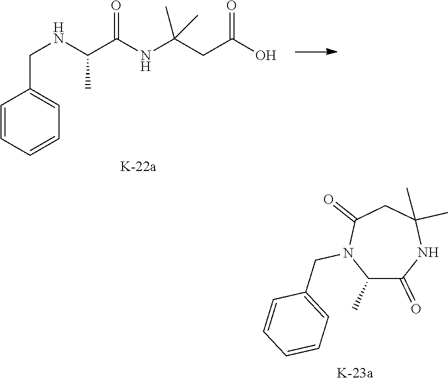 [SMILES: C[C@H](NCc1ccccc1)C(=O)NC(C)(C)CC(=O)O ; C[C@H]1C(=O)NC(C)(C)CC(=O)N1Cc1ccccc1]

To a stirred suspension of TBTU (25.95 g, 80.83 mmol, 1.5 eq.) in DCM (800 mL) is added a solution of intermediate K-22a (15.00 g, 53.89 mmol, 1.0 eq.) and DIPEA (28.73 mL, 161.67 mmol, 3.0 eq.) in DCM (800 mL) slowly at 0° C. and the reaction mixture is allowed to warm to rt and stirred for 16 h. The reaction mixture is quenched with water. The organic layer is separated, dried over Na₂SO₄, filtered, and concentrated under reduced pressure to get the crude product. The crude product is purified by column chromatography to give intermediate K-23a.

TABLE 4

| | | tᵣₑₜ | | |
|---|---|---|---|---|
| # | structure | [min] | [M + H]⁺ | HPLC method |
| K-23a | 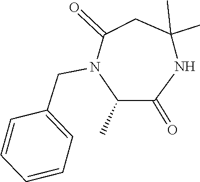 [SMILES: C[C@H]1C(=O)NC(C)(C)CC(=O)N1Cc1ccccc1] | 1.48 | 261 | X |

### Experimental Procedure for the Synthesis of K-24a

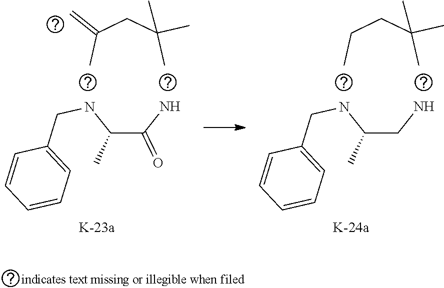

To a stirred solution of intermediate K-23a (9.00 g, 34.57 mmol, 1.0 eq.) in THF (50.00 mL) is added lithium aluminum hydride solution (5.26 mL, 138.28 mmol, 4.0 eq, 1 M in THF) at 0° C. and the reaction mixture is allowed to warm to rt and stirred at rt for 10 min. The reaction mixture is heated to 80° C. and stirred for 16 h. The reaction mixture is cooled to 0° C. and carefully quenched by slow addition of saturated Na₂SO₄ solution. The phases are separated, and the aqueous layer is extracted with EtOAc. The combined organic layer is dried over Na₂SO₄, filtered, and concentrated under reduced pressure. The crude is purified by column chromatography to yield intermediate K-24a.

TABLE 5

| | | tᵣₑₜ | | |
|---|---|---|---|---|
| # | structure | [min] | [M + H]⁺ | HPLC method |
| K-24a | 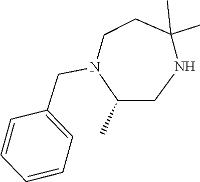 [SMILES: C[C@H]1CNC(C)(C)CCN1Cc1ccccc1] | 1.48 | 233 | X |

### Experimental Procedure for the Synthesis of K-25a

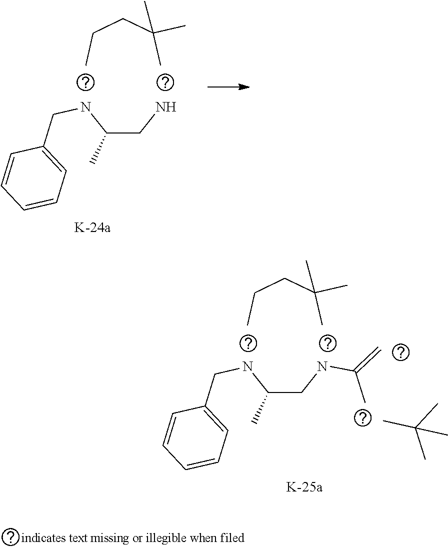

To a stirred solution of intermediate K-24a (1.00 g, 4.13 mmol, 1.0 eq.) in DCM (10 mL) is added NEt₃ (3.43 mL, 24.78 mmol, 6.0 eq.) and Boc anhydride (2.00 g, 9.08 mmol, 2.2 eq.). The reaction mixture is stirred at rt for 16 h. The reaction mixture is concentrated under reduced pressure, dissolved in acetonitrile and purified by chromatography to yield intermediate K-25a.

TABLE 6

| | | tᵣₑₜ | | HPLC |
|---|---|---|---|---|
| # | structure | [min] | [M + H]⁺ | method |
| K-25a | 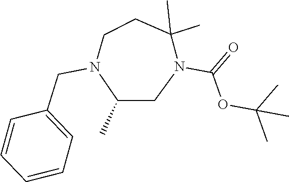 [SMILES: C[C@H]1CN(C(=O)OC(C)(C)C)C(C)(C)CCN1Cc1ccccc1] | 1.74 | 333 | X |

### Experimental Procedure for the Synthesis of K-26a

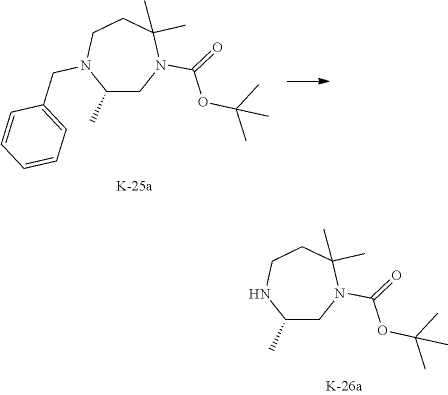 [SMILES: C[C@H]1CN(C(=O)OC(C)(C)C)C(C)(C)CCN1Cc1ccccc1 ; C[C@H]1CN(C(=O)OC(C)(C)C)C(C)(C)CCN1]

To a solution of intermediate K-25a (846 mg, 2.545 mmol, 1.0 eq) in MeOH (40 mL) in a hydrogenation reactor is added Pd/C (10%, 150.00 mg). The reaction mixture stirred under a pressure of 5 bar H₂ for 3 h. After complete conversion the reaction mixture is filtered, and the solvent is removed under reduced pressure to give intermediate K-26a.

TABLE 7

| | | tᵣₑₜ | | HPLC |
|---|---|---|---|---|
| # | structure | [min] | [M + H]+ | method |
| K-26a | 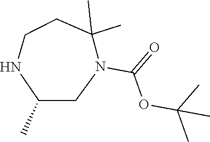 [SMILES: C[C@H]1CN(C(=O)OC(C)(C)C)C(C)(C)CCN1] | 1.09 | 243 | X |

### Experimental Procedure for the Synthesis of ethyl 1-methyl-2-oxocyclohexane-1-carboxylate

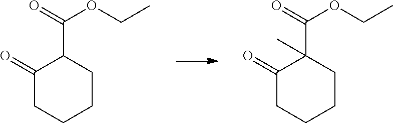

To a suspension of sodium hydride, (60% in mineral oil, 25.85 g, 646.3 mmol, 1.1 eq.) in THF (2.0 L) is added ethyl 2-oxocyclohexane-1-carboxylate (93.46 mL, 587.5 mmol, 1.0 eq.) dropwise at 0-10° C. The mixture is stirred at 10° C. for 30 min, then methyl iodide (55.11 mL, 881.3 mmol. 1.5 eq.) is added to the mixture dropwise at 10° C. The mixture is allowed to reach rt overnight. After complete conversion the reaction mixture is cooled to 0° C. and quenched with saturated aq. ammonium chloride solution. The product is extracted with EtOAc and the combined organic layers are washed with water and brine, dried over sodium sulfate and concentrated under reduced pressure to afford ethyl 1-methyl-2-oxocyclohexane-1-carboxylate which is used for the next step without further purification. (HPLC method A, tᵣₑₜ=1.14 min, [M+H]⁺=185).

### Experimental Procedure for the Synthesis of ethyl 2-(dicyanomethylidene)-1-methylcyclohexane-1-carboxylate

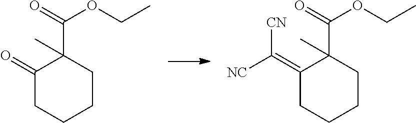 [SMILES: CCOC(=O)C1(C)CCCCC1=O ; CCOC(=O)C1(C)CCCCC1=C(C#N)C#N]

To a solution of ethyl 1-methyl-2-oxocyclohexane-1-carboxylate (108.0 g, 586.2 mmol) in toluene (1.03 L) is added malononitrile (58.04 g, 879.3 mmol, 1.5 eq.) followed by ammonium acetate (9.04 g, 117.2 mmol, 0.2 eq.) and acetic acid (13.41 mL, 234.5 mmol, 0.4 eq.) at rt. The mixture is stirred at 110° C. for 16 h. After complete conversion the mixture is diluted with EtOAc and washed with water and brine, dried over sodium sulfate and concentrated under reduced pressure to afford the crude product ethyl 2-(dicyanomethylidene)-1-methylcyclohexane-1-carboxylate. This crude material is used for the next step without further purification (see also Naumann et al., Pharmazie 51 (1996), 4).

### Experimental Procedure for the Synthesis of ethyl 2-amino-3-cyano-4-methyl-4,5,6,7-tetrahydro-1-benzothiophene-4-carboxylate

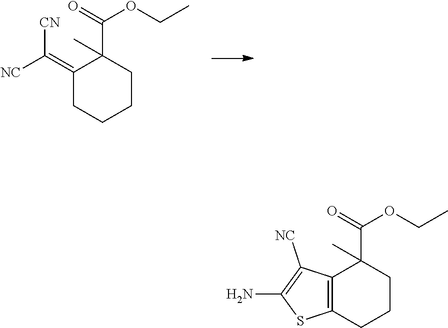 [SMILES: CCOC(=O)C1(C)CCCCC1=C(C#N)C#N ; CCOC(=O)C1(C)CCCc2sc(N)c(C#N)c21]

To a solution of ethyl 2-(dicyanomethylidene)-1-methylcyclohexane-1-carboxylate (250.0 g, 1.1 mol) in DMF (3.0 L) is added sulphur (68.9 g, 2.2 mol, 2.0 eq.) and L-proline (24.8 g, 0.22 mol, 0.2 eq.) and the resulting mixture is stirred at 80° C. for 12 h. After complete conversion the mixture is partitioned between EtOAc and water and the organic layer is collected. The aqueous layer is further extracted with EtOAc and the combined organic layers are washed with water and brine, dried over sodium sulfate and concentrated under reduced pressure to afford the crude product. The crude product is purified through column chromatography yielding ethyl 2-amino-3-cyano-4-methyl-4,5,6,7-tetrahydro-1-benzothiophene-4-carboxylate.

### Experimental Procedure for the Synthesis of 2-amino-3-cyano-4-methyl-4,5,6,7-tetrahydro-1-benzothiophene-4-carboxylic Acid

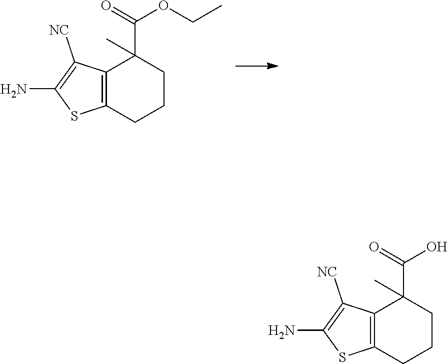 [SMILES: CCOC(=O)C1(C)CCCc2sc(N)c(C#N)c21 ; CC1(C(=O)O)CCCc2sc(N)c(C#N)c21]

Ethyl 2-amino-3-cyano-4-methyl-4,5,6,7-tetrahydro-1-benzothiophene-4-carboxylate (78.0 mg, 0.3 mmol, 1.0 eq.) is dissolved in EtOH (1.5 mL) and potassium hydroxide (4 M in water, 0.37 mL, 1.5 mmol, 5.0 eq.) is added. The mixture is stirred for 16 h at 78° C. After complete conversion, water and EtOAc is added to the reaction mixture, the pH of the aqueous phase is set to pH 4 using KHSO₄ solution (10% in water), and the product is extracted using EtOAc. The combined organic layers are dried, filtered and concentrated. The crude product is purified via acidic reversed phase chromatography (gradient elution: 20% to 90% acetonitrile in water) yielding 2-amino-3-cyano-4-methyl-4,5,6,7-tetrahydro-1-benzothiophene-4-carboxylic acid.

The crude product is purified by chromatography if necessary, enantiomers can be separated with preparative SFC.

TABLE 8

| | | | HPLC |
|---|---|---|---|
| structure | tᵣₑₜ [min] | [M + H]⁺ | method |
| 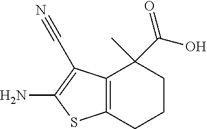 [SMILES: CC1(C(=O)O)CCCc2sc(N)c(C#N)c21] | 0.22 | 237 | A |
| 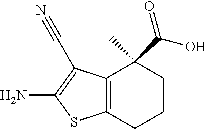 [SMILES: C[C@]1(C(=O)O)CCCc2sc(N)c(C#N)c21] | 0.25 | 237 | A |

### Experimental Procedure for the Synthesis of K-4a

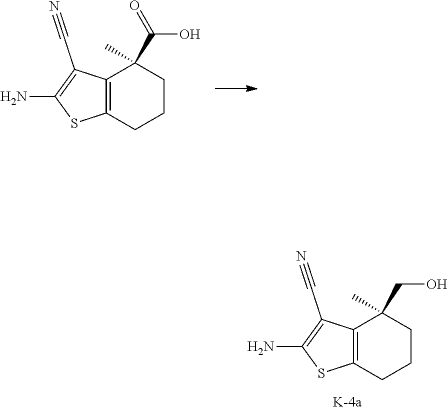 [SMILES: C[C@]1(C(=O)O)CCCc2sc(N)c(C#N)c21 ; C[C@]1(CO)CCCc2sc(N)c(C#N)c21]

To a solution 2-amino-3-cyano-4-methyl-4,5,6,7-tetrahydro-1-benzothiophene-4-carboxylic acid (22.00 g, 93.11 mmol, 1.0 eq.) in THF (300 mL) is added CDI (17.12 g, 102.42 mmol, 1.1 eq.) and the mixture is stirred at 50° C. for 1 h. The mixture is cooled to rt and sodium borohydride (10.78 g, 279.32 mmol, 3.0 eq.) suspended in 5 mL water is slowly added to the reaction mixture (exothermic reaction). After the addition the mixture is stirred for 1 h and subsequently quenched by slow addition of water (250 mL). The THF is removed under vacuum and the resulting mixture is extracted with EtOAc (3×120 mL). The combined organic layer is washed with water (3×100 mL) and the organic layer is dried with MgSO₄. The solvents are removed under vacuum and the crude product K-4a is used for the next steps without further purification.

The following intermediates K-4 (table 9) are available in an analogous manner starting from different intermediates. The crude product K-4 is purified by chromatography if necessary.

TABLE 9

| # | structure | tᵣₑₜ [min] | [M + H]⁺ | HPLC method |
|---|---|---|---|---|
| K-4a | 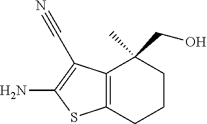 | 0.91 | 223 | Y |

### Experimental Procedure for the Synthesis of K-5a

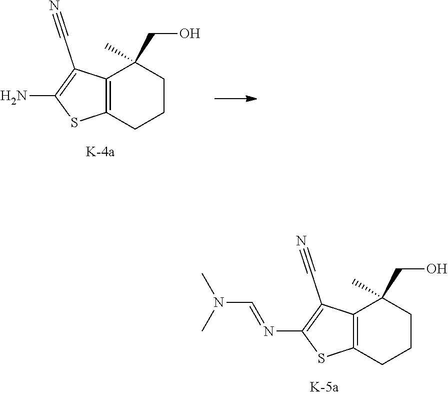 [SMILES: C[C@]1(CO)CCCc2sc(N)c(C#N)c21 ; CN(C)/C=N/c1sc2c(c1C#N)[C@@](C)(CO)CCC2]

K-4a (21.10 g, 75.93 mmol, 80% purity, 1.0 eq.) is mixed with N,N-dimethylformamide dimethyl acetal (57.6 g, 454.37 mmol, 94% purity, 6.0 eq.) and is irradiated in an ultrasound bath for 15 min until the mixture is a clear solution. Water (200 mL) is added and the reaction mixture is stirred for 30 min at rt until a precipitate forms. The precipitate is filtered and water (100 mL) is added. The mixture is irradiated in an ultrasound bath for 15 min and the precipitate is filtered. The precipitate is washed with isopropanol (25 mL) and dried under vacuum at 45° C. over night to give K-5a which is used for the next steps without further purification.

The following intermediates K-5 (table 10) are available in an analogous manner starting from different intermediates K-4. The crude product K-5 is purified by chromatography if necessary.

TABLE 10

| # | structure | tᵣₑₜ [min] | [M + H]⁺ | HPLC method |
|---|---|---|---|---|
| K-5a | 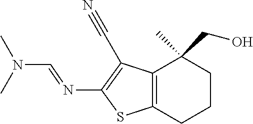 [SMILES: CN(C)/C=N/c1sc2c(c1C#N)[C@@](C)(CO)CCC2] | 1.11 | 278 | Y |

### Experimental Procedure for the Synthesis of K-6a

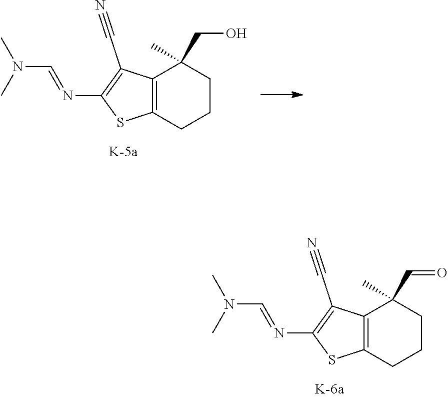 [SMILES: CN(C)/C=N/c1sc2c(c1C#N)[C@@](C)(CO)CCC2 ; CN(C)/C=N/c1sc2c(c1C#N)[C@@](C)(C=O)CCC2]

A solution of oxalyl chloride (12.2 mL, 144.20 mmol, 2.5 eq.) in DCM (120 mL) is cooled to −78° C. A solution of dry DMSO (18.44 mL, 259.57 mmol, 4.5 eq.) in DCM (60 mL) is added dropwise to the reaction mixture (exothermic reaction). The mixture is stirred for 30 min at −78° C. K-5a (16.00 g, 57.68 mmol, 1.0 eq.) is added slowly to the reaction mixture. The mixture is stirred for 30 min at −78° C. and trimethylamine (71.96 mL, 519.32 mmol, 9.0 eq.) is added dropwise. The reaction mixture is slowly warmed to rt and stirred for additional 2 h. Water and DCM is added to the mixture and the phases are separated. The aqueous layer is extracted two times with DCM and the combined organic layer is washed three times with water. The organic layer is dried with MgSO₄ and the solvents are removed under vacuum to give crude intermediate K-6a which is used without further purification in the next steps.

The following intermediates K-6 (table 11) are available in an analogous manner starting from different intermediates K-5. The crude product K-6 is purified by chromatography if necessary.

TABLE 11

| | | tᵣₑₜ | | HPLC |
|---|---|---|---|---|
| # | structure | [min] | [M + H]⁺ | method |
| K-6a | 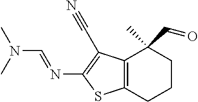 [SMILES: CN(C)/C=N/c1sc2c(c1C#N)[C@@](C)(C=O)CCC2] | 1.21 | 276 | Y |

### Experimental Procedure for the Synthesis of K-7a

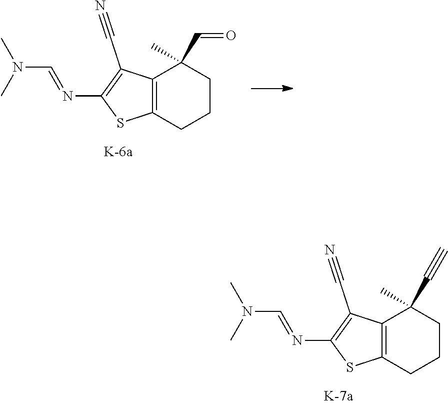 [SMILES: CN(C)/C=N/c1sc2c(c1C#N)[C@@](C)(C=O)CCC2 ; C#C[C@@]1(C)CCCc2sc(/N=C/N(C)C)c(C#N)c21]

A mixture of K-6a (15.90 g, 57.75 mmol, 1.0 eq.), Cs₂CO₃ (22.58 g, 69.26 mmol, 1.2 eq.) and MeOH (120 mL) is cooled to 0° C. and a solution of Bestmann-Ohira reagent (dimethyl (1-diazo-2-oxopropyl)phosphonate; 12.20 g, 63.52 mmol, 1.1 eq.) in MeOH (5 mL) is added dropwise to the reaction mixture. After 3 h at 0° C. the reaction mixture is slowly warmed to rt. After full conversion, the MeOH is removed under vacuum and water (500 mL) and EtOAc (500 mL) are added to the mixture. The phases are separated, and the aqueous layer is extracted two times with EtOAc. The combined organic layer is washed with water three times and dried over MgSO₄ and the solvents are removed under vacuum. The residue is mixed with diethyl ether and stirred for 30 min at rt. The mixture is cooled to 0° C. and stirred for additional 30 min before it is filtered and washed with small amounts of cold diethyl ether. The precipitate is dried under vacuum at 45° C. to give intermediate K-7a which is used for the next steps without further purification.

The following intermediates K-7 (table 12) are available in an analogous manner starting from different intermediates K-6. The crude product K-7 is purified by chromatography if necessary.

TABLE 12

| | | tᵣₑₜ | | HPLC |
|---|---|---|---|---|
| # | structure | [min] | [M + H]⁺ | method |
| K-7a | 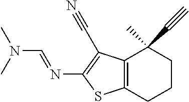 [SMILES: C#C[C@@]1(C)CCCc2sc(/N=C/N(C)C)c(C#N)c21] | 1.33 | 272 | Y |

### Experimental Procedure for the Synthesis of tert-butyl (2S)-2-[methoxy(methyl)carbamoyl]pyrrolidine-1-carboxylate

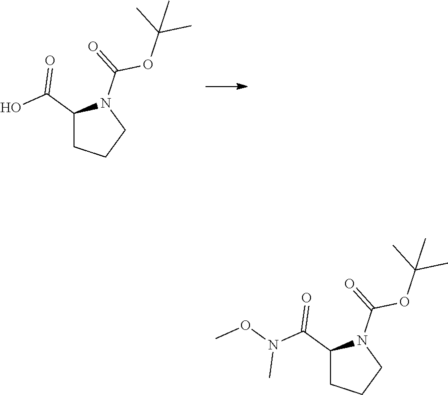 [SMILES: CC(C)(C)OC(=O)N1CCC[C@H]1C(=O)O ; CON(C)C(=O)[C@@H]1CCCN1C(=O)OC(C)(C)C]

To a stirred solution of Boc-proline (25 g, 116.15 mmol, 1.0 eq) and N,O-dimethylhydroxylamine hydrochloride (20.39 g, 209.06 mmol, 1.8 eq) in DMF (400 ml) is added EDCl hydrochloride (33.4 g, 174.2 mmol, 1.5 eq), HOBT (26.68 g, 174.2 mmol, 1.5 eq) and TEA (35.25 mg, 348.44 mmol, 3.0 eq. The reaction mixture is stirred at 100° C. for 3 h. After complete conversion the reaction mixture is concentrated under reduced pressure, diluted with water (250 ml) and a saturated sodium bicarbonate solution (250 ml). The mixture is extracted EtOAc and the separated organic phase is washed with a saturated sodium bicarbonate solution (3*500 ml). The organic phase is dried over magnesium sulfate, filtered and concentrated under reduced pressure to get the crude product as a light yellow oil. (HPLC method A; tᵣₑₜ=1.03 min; [M+H]⁺=281)

### Experimental Procedure for the Synthesis of tert-butyl (2S)-2-acetylpyrrolidine-1-carboxylate

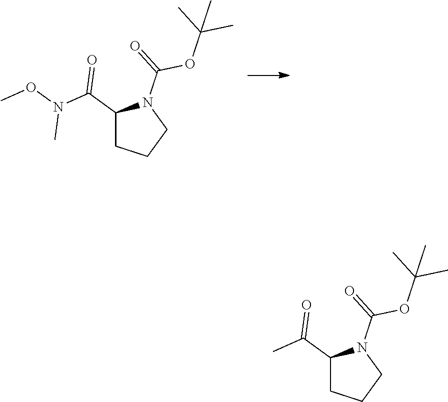 [SMILES: CON(C)C(=O)[C@@H]1CCCN1C(=O)OC(C)(C)C ; CC(=O)[C@@H]1CCCN1C(=O)OC(C)(C)C]

To the crude product from the previous step (26.83 g, 103.9 mmol, 1.0 eq) is dissolved in THF (400 ml) is added at 0° C. methylmagnesium bromide (3.4 M in THF, 109.2 g, 332.37 mmol, 3.2 eq) and stirred for 2 h. After complete conversion the reaction mixture is quenched by addition of a saturated sodium chloride solution (500 ml) and extracted with EtOAc (3*500 ml). The combined organic phases are dried over sodium sulfate, filtered and concentrated under reduced pressure to get the crude product as a light yellow oil. (HPLC method A; tᵣₑₜ=1.05 min; [M+H-Boc]⁺=114)

### Experimental Procedure for the Synthesis of tert-butyl (2S)-2-[(1S)-1-hydroxyethyl]-pyrrolidine-1-carboxylate

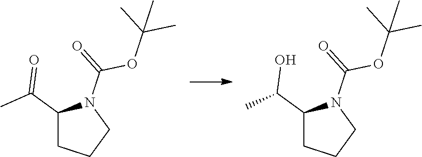

To a solution of (R)-Methyl oxazaborolidine (5.427 g, 19.58 mmol, 0.2 eq) in THF (200 ml) is added borane-dimethyl sulfide complex (2 M in THF, 54.41 g, 127.27 mmol, 1.3 eq) and stirred at rt for 1 h. To the reaction mixture a solution of (S)-tert-butyl 2-acetylpyrrolidine-1-carboxylate (20.88 g, 97.9 mmol, 1.0 eq) dissolved in THF (100 ml) and added slowly and the reaction mixture is stirred at rt for 1 h. After complete conversion the reaction mixture is carefully quenched with MeOH and concentrated under reduced pressure. The crude is purified by NP column chromatography using Cyclohexane/EtOAc as eluent (15-45%). The product containing fractions are combined and concentrated under reduced pressure to yield tert-butyl (2S)-2-[(1S)-1-hydroxyethyl]pyrrolidine-1-carboxylate. (HPLC method A; tᵣₑₜ=1.11 min; [M+H]⁺=238)

### Experimental Procedure for the Synthesis of (1S)-1-[(2S)-1-methylpyrrolidin-2-yl]ethan-1-ol

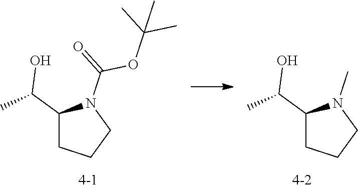

Tert-butyl (2S)-2-[(1S)-1-hydroxyethyl]pyrrolidine-1-carboxylate (15.36 g, 71.35 mmol, 1.0 eq) is treated with a lithium aluminium hydride solution (2 M in THF, 97.4 g, 214 mmol, 3.0 eq) and stirred at 80° C. for 1 h. After complete conversion the reaction mixture is quenched under cooling in an ice bath by addition of water (4 ml). The precipitated Li-salts are removed, the organic phase is dried over sodium sulfate, filtered and carefully concentrated at 100 mbar pressure at 45° C. bath temperature to yield the product (1S)-1-[(2S)-1-methylpyrrolidin-2-yl]ethan-1-ol as a colorless oil. (HPLC method A, tᵣₑₜ=0.2 min; [M+H]⁺=130)

### Experimental Procedure for the Synthesis of 6-[(2S)-2,4-dimethylpiperazin-1-yl]pyridine-2-carbonitrile

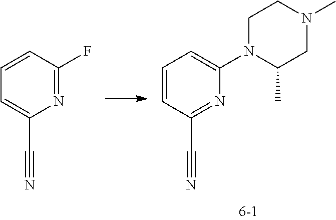 [SMILES: N#Cc1cccc(F)n1 ; C[C@H]1CN(C)CCN1c1cccc(C#N)n1]

To a solution of (S)-1,3-dimethyl-piperazine (280.6 mg, 2.46 mmol, 2.0 eq.) and 2-cyano-6-fluoropyridine (150 mg, 1.23 mmol, 1.0 eq) in NMP is added DIPEA (398.9 mg, 3.07 mmol, 2.5 eq.) at rt. The reaction mixture is stirred at 80° C. overnight. After complete conversion the reaction mixture is cooled to rt and water and ACN is added. This crude material is purified by column chromatography to afford 6-[(2S)-2,4-dimethylpiperazin-1-yl]pyridine-2-carbonitrile. (HPLC method A; tᵣₑₜ=1.16 min; [M+H]⁺=217)

### Experimental Procedure for the Synthesis of tert-butyl (3S)-4-(4-cyanopyrimidin-2-yl)-3-methyl-1,4-diazepane-1-carboxylate

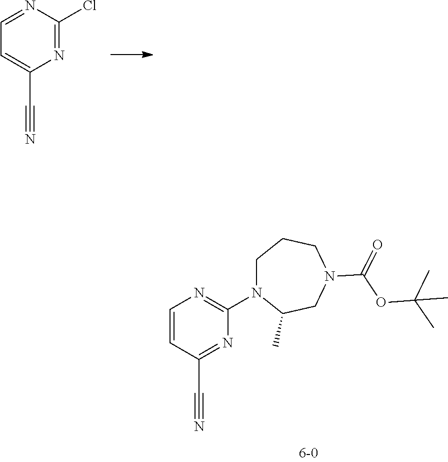 [SMILES: N#Cc1ccnc(Cl)n1 ; C[C@H]1CN(C(=O)OC(C)(C)C)CCCN1c1nccc(C#N)n1]

To a solution of (S)-tert-butyl-3-methyl-1,4-diazepane-1-carboxylate (846.0 mg, 214.30 mmol, 1.0 eq.) and 2-chloropyrimidine-4-carbonitrile (528.9 mg, 139.54 mmol, 1.0 eq) in DMSO (4 ml, 4,5 V) is added TEA (1.1 ml, 101.19 mmol, 2.0 eq.) at rt. The reaction mixture is stirred at 80° C. for 1 h. After complete conversion the reaction mixture is cooled to rt and water and EtOAc is added. The phases are separated. The organic layer is washed with water, dried over sodium sulfate, then filtered and concentrated under reduced pressure to the get crude product. The crude product is purified by chromatography if necessary. (HPLC method A; tᵣₑₜ=1.38 min; [M+H-Isobuten]⁺=262)

### Experimental Procedure for the Synthesis of tert-butyl (2S)-2-[(1S)-1-[(4-cyanopyrimidin-2-yl)oxy]ethyl]pyrrolidine-1-carboxylate

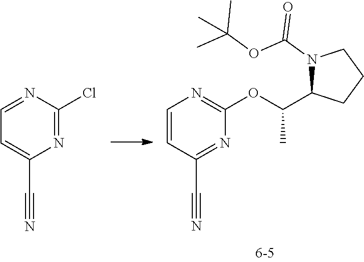 [SMILES: N#Cc1ccnc(Cl)n1 ; C[C@H](Oc1nccc(C#N)n1)[C@@H]1CCCN1C(=O)OC(C)(C)C]

To a stirred solution of 2-chloropyrimidine-4-carbonitrile (5.00 g, 35.83 mmol, 1.0 eq) and (S)-2-((S)-1-hydroxy-ethyl)-pyrrolidine-1-carboxylic acid tert-butyl-ester (9.25 g, 42.99 mmol, 1.2 eq) in DMA (20 mL) is added cesium carbonate (23.35 g, 71.66 mmol, 2.0 eq) at rt. The reaction mixture is stirred at rt for 3 h. After complete conversion the reaction mixture is diluted with EtOAc, washed with water and brine, dried over sodium sulfate, filtered and concentrate under reduced pressure to get the crude product, which is purified by column chromatography to afford tert-butyl (2S)-2-[(1S)-1-[(4-cyanopyrimidin-2-yl)oxy]ethyl]-pyrrolidine-1-carboxylate. (HPLC method A; tᵣₑₜ=1.95 min; [M+H]⁺=219).

### Experimental Procedure for the Synthesis of 2-[(1S)-1-[(2S)-1-methylpyrrolidin-2-yl]ethoxy]pyrimidine-4-carbonitrile

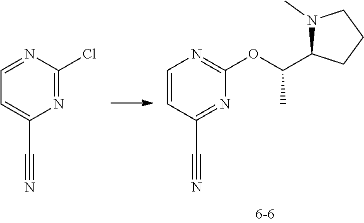 [SMILES: N#Cc1ccnc(Cl)n1 ; C[C@H](Oc1nccc(C#N)n1)[C@@H]1CCCN1C]

To a solution of 2,4-dichloropyrimidine (2.5 g, 17.91 mmol, 1.0 eq) and (1S)-1-[(2S)-1-methylpyrrolidin-2-yl]ethanol (2.8 g, 21.67 mmol, 1.2 eq) in ACN (50.0 ml, 20V) is added potassium carbonate (3.7 g, 26.87 mmol, 1.5 eq). The reaction is stirred at 60° C. for 3 hours. After complete conversion the reaction is filtered and evaporated to afford the crude product which is purified through flash chromatography. (HPLC method A, tᵣₑₜ=1.03 min; [M+H]⁺=233) The following intermediates are available in an analogous manner using different nitriles.

TABLE 13

| | | tᵣₑₜ | | HPLC |
|---|---|---|---|---|
| | structure | [min] | [M + H]⁺ | method |
| 6-0 | 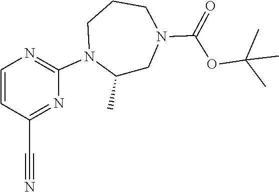 [SMILES: C[C@H]1CN(C(=O)OC(C)(C)C)CCCN1c1nccc(C#N)n1] | 1.38 | 262 (- iso-butene) | A |
| 6-1 | 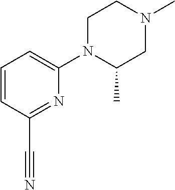 [SMILES: C[C@H]1CN(C)CCN1c1cccc(C#N)n1] | 1.16 | 217 | A |
| 6-2 | 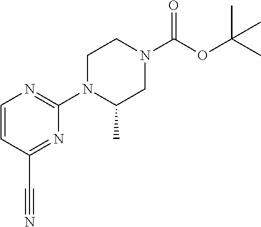 [SMILES: C[C@H]1CN(C(=O)OC(C)(C)C)CCN1c1nccc(C#N)n1] | 1.41 | 248 | A |
| 6-3 | 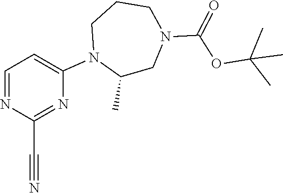 [SMILES: C[C@H]1CN(C(=O)OC(C)(C)C)CCCN1c1ccnc(C#N)n1] | 1.23 | 318 | A |
| 6-4 | 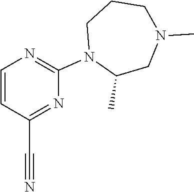 [SMILES: C[C@H]1CN(C)CCCN1c1nccc(C#N)n1] | 1.05 | 238 | A |
| 6-5 | 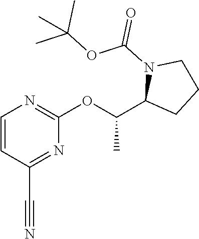 [SMILES: C[C@H](Oc1nccc(C#N)n1)[C@@H]1CCCN1C(=O)OC(C)(C)C] | 1.35 | 219 (- Boc) | A |
| 6-6 | 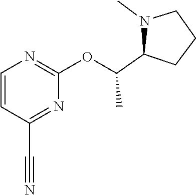 [SMILES: C[C@H](Oc1nccc(C#N)n1)[C@@H]1CCCN1C] | 1.94 | 233 | A |
| 6-7 | 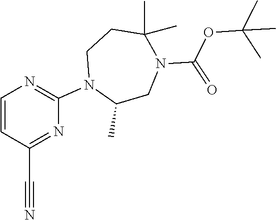 [SMILES: C[C@H]1CN(C(=O)OC(C)(C)C)C(C)(C)CCN1c1nccc(C#N)n1] | 2.39 | 346 | Z |
| 6-8 | 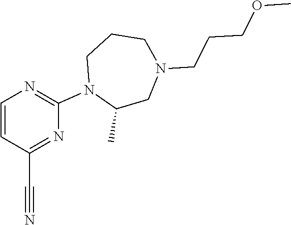 [SMILES: COCCCN1CCCN(c2nccc(C#N)n2)[C@@H](C)C1] | — | — | — |
| 6-9 | 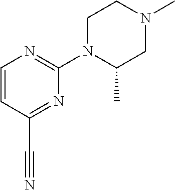 [SMILES: C[C@H]1CN(C)CCN1c1nccc(C#N)n1] | 0.9 | 218 | A |
| 6-10 | 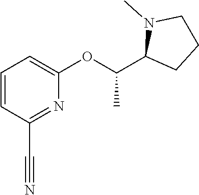 [SMILES: C[C@H](Oc1cccc(C#N)n1)[C@@H]1CCCN1C] | — | — | — |
| 6-11 | 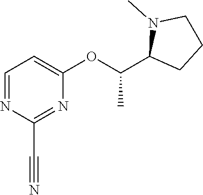 [SMILES: C[C@H](Oc1ccnc(C#N)n1)[C@@H]1CCCN1C] | 1.14 | 233 | A |

### Experimental Procedure for the Synthesis of tert-butyl (3S)-4-{4-[(Z)—N′-hydroxycarbamimidoyl]pyrimidin-2-yl}-3-methyl-1,4-diazepane-1-carboxylate

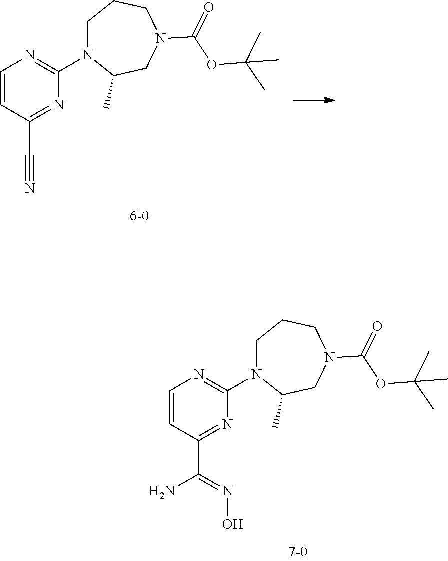 [SMILES: C[C@H]1CN(C(=O)OC(C)(C)C)CCCN1c1nccc(C#N)n1 ; C[C@H]1CN(C(=O)OC(C)(C)C)CCCN1c1nccc(/C(N)=N/O)n1]

To a solution of tert-butyl (3S)-4-(4-cyanopyrimidin-2-yl)-3-methyl-1,4-diazepane-1-carboxylate (33.85 g, 106.65 mmol, 1.0 eq) in EtOH (270 ml, 8.0 V) is added hydroxylamine solution 50% in water (13.05 ml, 213.30 mmol, 2.0 eq) at rt. The reaction mixture is stirred at 60° C. for 1 h. After complete conversion the reaction mixture is concentrated under reduced pressure to afford tert-butyl (3S)-4-{4-[(Z)—N′-hydroxycarbamimidoyl]pyrimidin-2-yl}-3-methyl-1,4-diazepane-1-carboxylate which is used for the next step without further purification. (HPLC method A; tᵣₑₜ=1.12 min; [M+H]⁺=351)

### Experimental Procedure for the Synthesis of 6-fluoro-N′-hydroxypyridine-2-carboximidamide

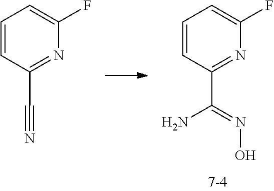 [SMILES: N#Cc1cccc(F)n1 ; N/C(=N\O)c1cccc(F)n1]

To a solution of 2-cyano-6-fluoropyridine (Enamine EN-300-99870) (1.07 g, 8.325 mmol, 1.0-eq) in EtOH is added hydroxylamine solution 50% in water (1.1 g, 16.65 mmol, 2.0 eq) at rt. The reaction mixture is stirred at 80° C. for 10 min. After complete conversion the reaction mixture is concentrated under reduced pressure to afford 6-fluoro-N′-hydroxypyridine-2-carboximidamide which is used for the next step without further purification.

The following intermediates are available in an analogous manner.

TABLE 14

| | | tᵣₑₜ | | HPLC |
|---|---|---|---|---|
| | Structure | [min] | [M + H]⁺ | method |
| 7-0 | 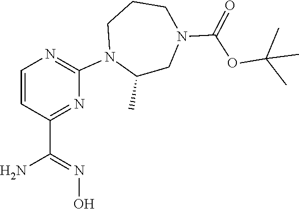 [SMILES: C[C@H]1CN(C(=O)OC(C)(C)C)CCCN1c1nccc(/C(N)=N/O)n1] | 1.12 | 351 | A |
| 7-1 | 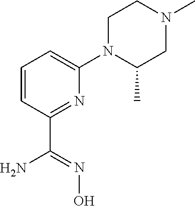 [SMILES: C[C@H]1CN(C)CCN1c1cccc(/C(N)=N/O)n1] | 0.30 | 250 | E |
| 7-2 | 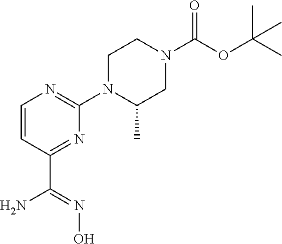 [SMILES: C[C@H]1CN(C(=O)OC(C)(C)C)CCN1c1nccc(/C(N)=N/O)n1] | 1.14 | 337 | A |
| 7-3 | 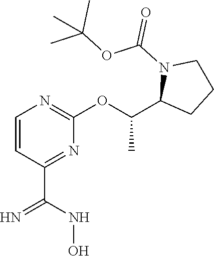 [SMILES: C[C@H](Oc1nccc(C(=N)NO)n1)[C@@H]1CCCN1C(=O)OC(C)(C)C] | 1.73 | 352 | A |
| 7-4 | 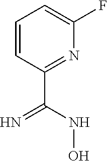 [SMILES: N=C(NO)c1cccc(F)n1] | 0.31 | 156 | A |
| 7-5 | 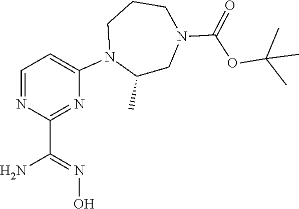 [SMILES: C[C@H]1CN(C(=O)OC(C)(C)C)CCCN1c1ccnc(/C(N)=N/O)n1] | 1.00 | 351 | A |
| 7-6 | 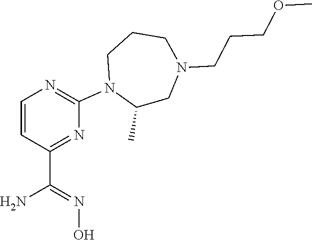 [SMILES: COCCCN1CCCN(c2nccc(/C(N)=N/O)n2)[C@@H](C)C1] | 0.39 | 323 | E |
| 7-7 | 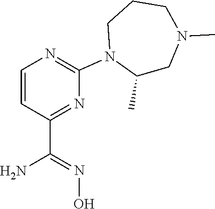 [SMILES: C[C@H]1CN(C)CCCN1c1nccc(/C(N)=N/O)n1] | 0.70 | 265 | A |
| 7-8 | 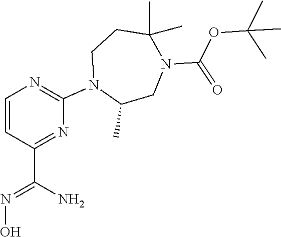 [SMILES: C[C@H]1CN(C(=O)OC(C)(C)C)C(C)(C)CCN1c1nccc(/C(N)=N/O)n1] | 2.22 | 379 | G |
| 7-9 | 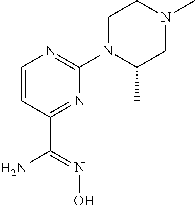 [SMILES: C[C@H]1CN(C)CCN1c1nccc(/C(N)=N/O)n1] | 0.76 | 251 | A |
| 7-10 | 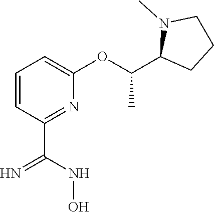 [SMILES: C[C@H](Oc1cccc(C(=N)NO)n1)[C@@H]1CCCN1C] | — | — | — |
| 7-11 | 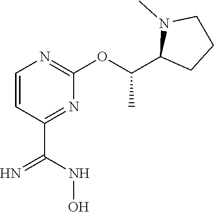 [SMILES: C[C@H](Oc1nccc(C(=N)NO)n1)[C@@H]1CCCN1C] | 0.77 | 266 | A |
| 7-12 | 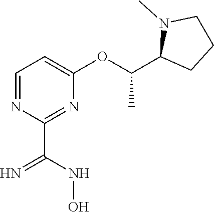 [SMILES: C[C@H](Oc1ccnc(C(=N)NO)n1)[C@@H]1CCCN1C] | 0.32 | 266 | E |

### Experimental Procedure for the Synthesis of tert-butyl (3S)-4-{4-[(Z)—N′—[(Z)-(4S)-2-amino-3-cyano-4-methyl-4,5,6,7-tetrahydro-1-benzothiophene-4-carbonyloxy]carbamimidoyl]-pyrimidin-2-yl}-3-methyl-1,4-diazepane-1-carboxylate

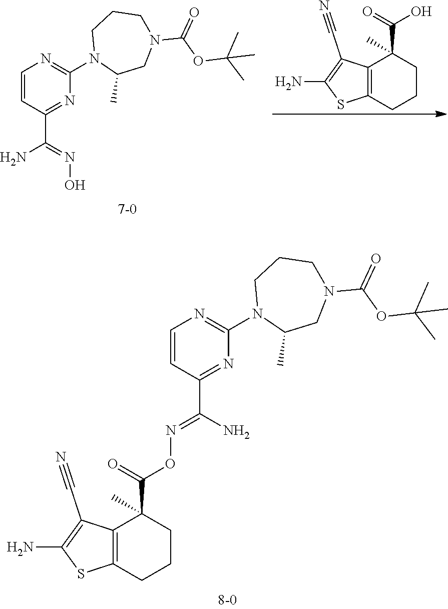 [SMILES: C[C@H]1CN(C(=O)OC(C)(C)C)CCCN1c1nccc(/C(N)=N/O)n1 ; C[C@]1(C(=O)O)CCCc2sc(N)c(C#N)c21 ; C[C@H]1CN(C(=O)OC(C)(C)C)CCCN1c1nccc(/C(N)=N/OC(=O)[C@@]2(C)CCCc3sc(N)c(C#N)c32)n1]

To a stirred solution of (4S)-2-amino-3-cyano-4-methyl-4,5,6,7-tetrahydro-1-benzo-thiophene-4-carboxylic acid (2.53 g, 10.70 mmol, 1.0 eq) in DMSO (10 ml) are added TEA (2.17 g, 21.40 mmol, 2.0 eq) and O-(7-Azabenzotriazol-1-yl)-N,N,N′,N′-tetramethyluronium-hexafluorphosphate (HATU, 4.27 g, 11.24 mmol, 1.10 eq) at rt. The mixture is stirred for 15 min at rt. Tert-butyl (3S)-4-{4-[(Z)—N′-hydroxycarbamimidoyl]pyrimidin-2-yl}-3-methyl-1,4-diazepane-1-carboxylate (3.75 g, 10.70 mmol, 1.0 eq) is added at rt and stirred overnight. After complete conversion the reaction mixture is diluted with water and EtOAc. The phases are separated. The organic layer is washed with water, dried over sodium sulfate, filtered and concentrated under reduced pressure to get the crude product. This crude material is purified by column chromatography (gradient elution: 0-5% MeOH in DCM) to afford tert-butyl (3S)-4-{4-[(Z)—N′—[(Z)-(4S)-2-amino-3-cyano-4-methyl-4,5,6,7-tetrahydro-1-benzothio-phene-4-carbonyloxy]carbamimidoyl]pyrimidin-2-yl}-3-methyl-1,4-diazepane-1-carboxy-late. (HPLC method A; tᵣₑₜ=1.43 min; [M+H]⁺=513)

The following intermediates are available in an analogous manner.

TABLE 15

| | | tᵣₑₜ | | HPLC |
|---|---|---|---|---|
| | Structure | [min] | [M + H]⁺ | method |
| 8-0 | 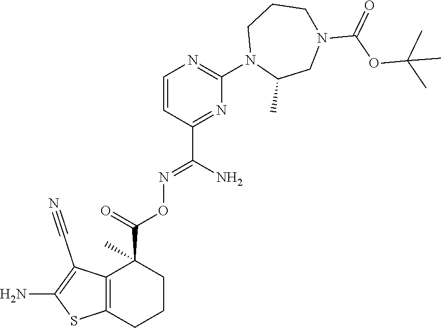 [SMILES: C[C@H]1CN(C(=O)OC(C)(C)C)CCCN1c1nccc(/C(N)=N/OC(=O)[C@@]2(C)CCCc3sc(N)c(C#N)c32)n1] | 1.43 | 512 (-isobutene) | A |
| 8-1 | 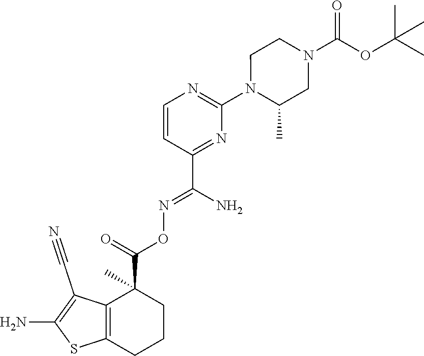 [SMILES: C[C@H]1CN(C(=O)OC(C)(C)C)CCN1c1nccc(/C(N)=N/OC(=O)[C@@]2(C)CCCc3sc(N)c(C#N)c32)n1] | 1.42 | 555 | A |
| 8-2 | 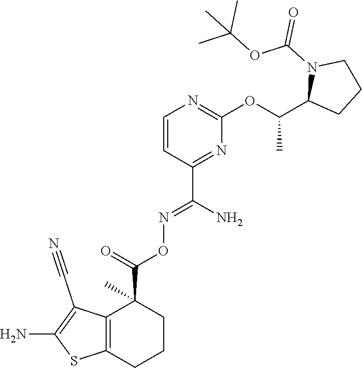 [SMILES: C[C@H](Oc1nccc(/C(N)=N/OC(=O)[C@@]2(C)CCCc3sc(N)c(C#N)c32)n1)[C@@H]1CCCN1C(=O)OC(C)(C)C] | 1.92 | 570 | A |
| 8-3 | 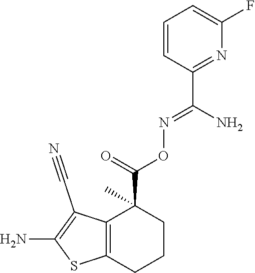 [SMILES: C[C@]1(C(=O)O/N=C(\N)c2cccc(F)n2)CCCc2sc(N)c(C#N)c21] | 1.16 | 374 | A |
| 8-4 | 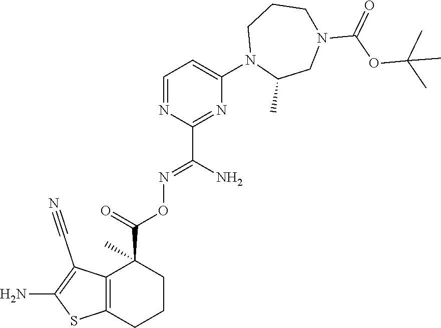 [SMILES: C[C@H]1CN(C(=O)OC(C)(C)C)CCCN1c1ccnc(/C(N)=N/OC(=O)[C@@]2(C)CCCc3sc(N)c(C#N)c32)n1] | 1.28 | 569 | A |
| 8-5 | 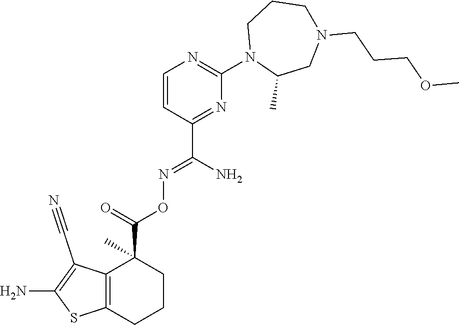 [SMILES: COCCCN1CCCN(c2nccc(/C(N)=N/OC(=O)[C@@]3(C)CCCc4sc(N)c(C#N)c43)n2)[C@@H](C)C1] | 0.64 | 541 | E |
| 8-6 | 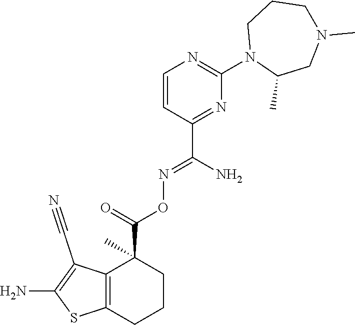 [SMILES: C[C@H]1CN(C)CCCN1c1nccc(/C(N)=N/OC(=O)[C@@]2(C)CCCc3sc(N)c(C#N)c32)n1] | 1.26 | 483 | A |
| 8-7 | 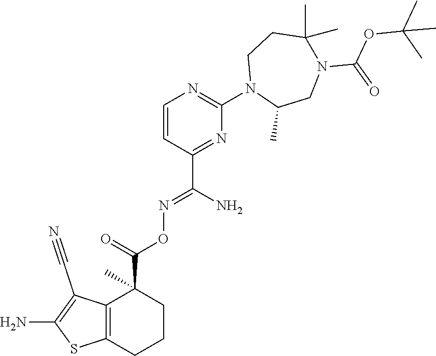 [SMILES: C[C@H]1CN(C(=O)OC(C)(C)C)C(C)(C)CCN1c1nccc(/C(N)=N/OC(=O)[C@@]2(C)CCCc3sc(N)c(C#N)c32)n1] | 2.18 | 597 | G |
| 8-8 | 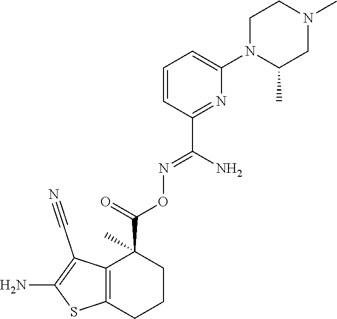 [SMILES: C[C@H]1CN(C)CCN1c1cccc(/C(N)=N/OC(=O)[C@@]2(C)CCCc3sc(N)c(C#N)c32)n1] | 1.27 | 468 | A |
| 8-9 | 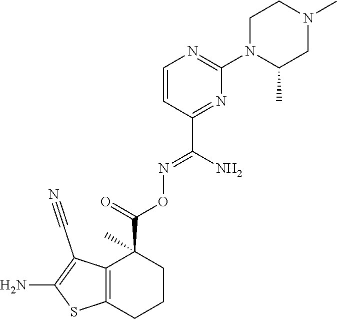 [SMILES: C[C@H]1CN(C)CCN1c1nccc(/C(N)=N/OC(=O)[C@@]2(C)CCCc3sc(N)c(C#N)c32)n1] | 1.28 | 469 | A |
| 8-10 | 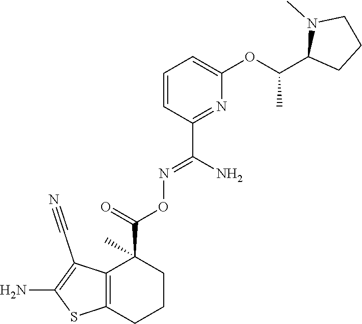 [SMILES: C[C@H](Oc1cccc(/C(N)=N/OC(=O)[C@@]2(C)CCCc3sc(N)c(C#N)c32)n1)[C@@H]1CCCN1C] | — | — | — |
| 8-11 | 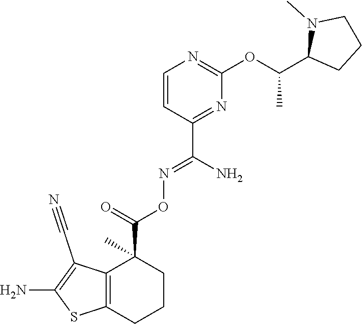 [SMILES: C[C@H](Oc1nccc(/C(N)=N/OC(=O)[C@@]2(C)CCCc3sc(N)c(C#N)c32)n1)[C@@H]1CCCN1C] | 1.24 | 484 | A |
| 8-12 | 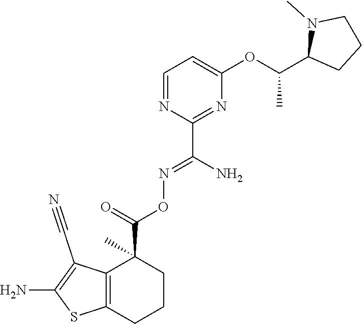 [SMILES: C[C@H](Oc1ccnc(/C(N)=N/OC(=O)[C@@]2(C)CCCc3sc(N)c(C#N)c32)n1)[C@@H]1CCCN1C] | 0.56 | 484 | E |

### Experimental Procedure for the Synthesis of V-15

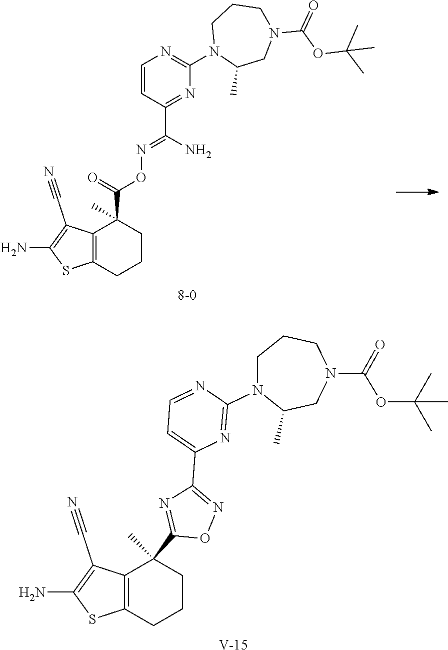

### Method A

To a stirred solution of tert-butyl (3S)-4-{4-[(Z)—N′—[(Z)-(4S)-2-amino-3-cyano-4-methyl-4,5,6,7-tetrahydro-1-benzothiophene-4-carbonyloxy]carbamimidoyl]pyrimidin-2-yl}-3-methyl-1,4-diazepane-1-carboxylate (2.35 g, 4.13 mmol, 1.0 eq) in THF (20 mL) is added tetrabutylammonium hydroxide (40 wt. % in water, 2.95 ml, 4.55 mmol, 1.1 eq) at rt and stirred for 30 minutes. After complete conversion the reaction mixture is diluted with water and EtOAc. The phases are separated. The organic layer is washed with water, dried over sodium sulfate, filtered and concentrated under reduced pressure to get crude product. This crude material is purified by column chromatography (gradient elution: 0-10% MeOH in DCM) to afford tert-butyl (3S)-4-(4-{5-[(4S)-2-amino-3-cyano-4-methyl-4,5,6,7-tetrahydro-1-benzothio-phen-4-yl]-1,2,4-oxadiazol-3-yl}pyrimidin-2-yl)-3-methyl-1,4-diazepane-1-carboxylate. (HPLC method A; tᵣₑₜ=1.51 min; [M+H]⁺=551)

### Method B

To a stirred solution of tert-butyl (3S)-4-{4-[(Z)—N′—[(Z)-(4S)-2-amino-3-cyano-4-methyl-4,5,6,7-tetrahydro-1-benzothiophene-4-carbonyloxy]carbamimidoyl]pyrimidin-2-yl}-3-methyl-1,4-diazepane-1-carboxylate (2.00 g, 3.51 mmol, 1.0 eq) in THF (40 mL) is added DBU (1.98 mL, 14.04 mmol, 4.0 eq) at rt. The reaction mixture is stirred at 70° C. overnight. After complete conversion the reaction mixture is concentrated under reduced pressure to get the crude product. The crude product is purified by column chromatography to afford tert-butyl (3S)-4-(4-{5-[(4S)-2-amino-3-cyano-4-methyl-4,5,6,7-tetrahydro-1-benzo-thiophen-4-yl]-1,2,4-oxadiazol-3-yl}pyrimidin-2-yl)-3-methyl-1,4-diazepane-1-carboxylate (HPLC method LCMS3, basisch_1; tᵣₑₜ=1.51 min; [M+H]⁺=551)

### Experimental Procedure for the Synthesis of (4S)-2-amino-4-methyl-4-(3-{2-[(2S)-2-methyl-1,4-diazepan-1-yl]pyrimidin-4-yl}-1,2,4-oxadiazol-5-yl)-4,5,6,7-tetrahydro-1-benzo-thiophene-3-carbonitrile

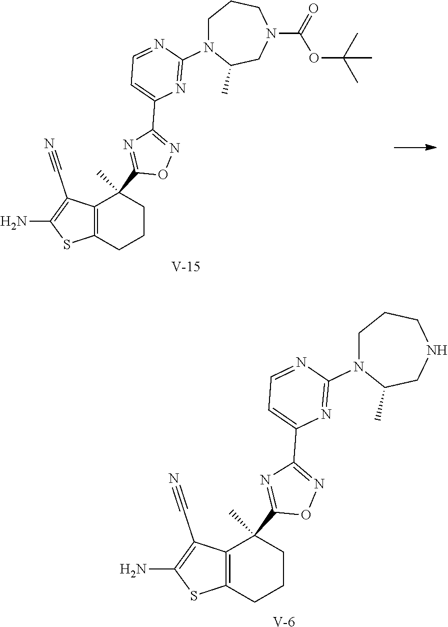

To a stirred solution of tert-butyl (3S)-4-(4-{5-[(4S)-2-amino-3-cyano-4-methyl-4,5,6,7-tetrahydro-1-benzothiophene-4-yl]-1,2,4-oxadiazol-3-yl}pyrimidin-2-yl)-3-methyl-1,4-diazepane-1-carboxylate (20.00 g, 34.52 mmol, 1.0 eq) in MeOH (350 mL, 18.4 V) is added conc. HCl (32.88 mL, 345.21 mmol, 10.0 eq) at rt. The reaction mixture is stirred at 50° C. for 2 h. After complete conversion the reaction mixture is concentrated under reduced pressure and diluted with water. The aqueous phase is extracted with DCM. The combined organic layers are dried over sodium sulfate, filtered and concentrated under reduced pressure to afford (4S)-2-amino-4-methyl-4-(3-{2-[(2S)-2-methyl-1,4-diazepan-1-yl]-pyrimidin-4-yl}-1,2,4-oxadiazol-5-yl)-4,5,6,7-tetrahydro-1-benzothiophene-3-carbonitrile which is used for the next step without further purification. (HPLC method A; tᵣₑₜ=1.21 min; [M+H]⁺=451)

### Experimental Procedure for the Synthesis of (4S)-2-amino-4-(3-{2-[(2S)-4-(cyclopropylmethyl)-2-methyl-1,4-diazepan-1-yl]pyrimidin-4-yl}-1,2,4-oxadiazol-5-yl)-4-methyl-4,5,6,7-tetrahydro-1-benzothiophene-3-carbonitrile

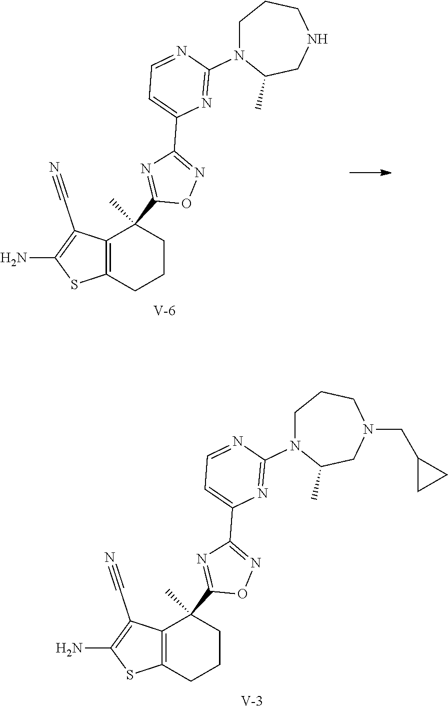

To a stirred solution of (4S)-2-amino-4-methyl-4-(3-{2-[(2S)-2-methyl-1,4-diazepan-1-yl]pyrimidin-4-yl}-1,2,4-oxadiazol-5-yl)-4,5,6,7-tetrahydro-1-benzothiophene-3-carbonitrile (30 mg, 0.057 mmol, 1.0 eq) in DMF (1 ml, 33.3 V) is added cyclopropanecarboxaldehyde (4.02 mg, 0.057 mmol, 1.0 eq) at rt. The reaction mixture is stirred at rt for 30 minutes before sodium triacetoxyborohydride (36.44 mg, 0.172 mmol, 3.0 eq) is added and the reaction mixture is stirred for additional 30 min at rt. After complete conversion the reaction mixture is diluted with water and purified by reverse phase column chromatography to afford (4S)-2-amino-4-(3-{2-[(2S)-4-(cyclopropylmethyl)-2-methyl-1,4-diazepan-1-yl]pyrimidin-4-yl}-1,2,4-oxadiazol-5-yl)-4-methyl-4,5,6,7-tetrahydro-1-benzo-thiophene-3-carbonitrile.

### Experimental Procedure for the Synthesis of (4S)-2-amino-4-methyl-4-(3-{2-[(2S)-2-methyl-4-(propan-2-yl)-1,4-diazepan-1-yl]pyrimidin-4-yl}-1,2,4-oxadiazol-5-yl)-4,5,6,7-tetrahydro-1-benzothiophene-3-carbonitrile

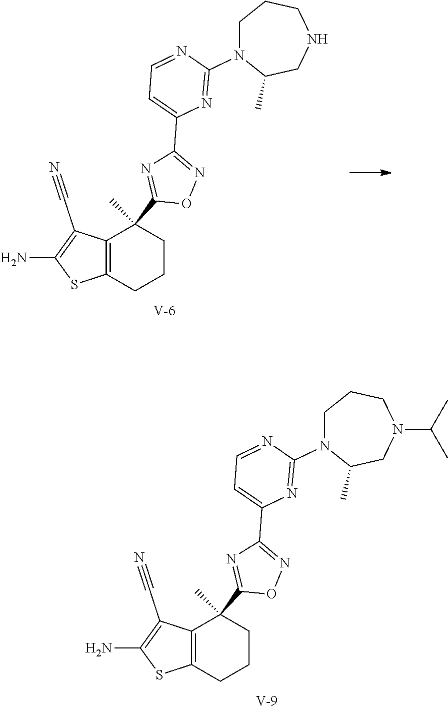

To a stirred solution of (4S)-2-amino-4-methyl-4-(3-{2-[(2S)-2-methyl-1,4-diazepan-1-yl]pyrimidin-4-yl}-1,2,4-oxadiazol-5-yl)-4,5,6,7-tetrahydro-1-benzothiophene-3-carbonitrile (1.5 g, 3.33 mmol, 1.0 eq) and potassium carbonate (2.3 g, 16.65 mmol, 5.0 eq) in ACN (25 ml, 16.7 V) is added 2-iodopropane (622.5 mg, 3.66 mmol, 1.1 eq) at rt. The reaction mixture is stirred at 45° C. for 24 h. After complete conversion the reaction mixture is diluted with water. The aqueous phase is extracted with EtOAc. The combined organic layers are dried over sodium sulfate, filtered and concentrated under reduced pressure and purified by normal phase column chromatography to afford (4S)-2-amino-4-methyl-4-(3-{2-[(2S)-2-methyl-4-(propan-2-yl)-1,4-diazepan-1-yl]pyrimidin-4-yl}-1,2,4-oxadiazol-5-yl)-4,5,6,7-tetrahydro-1-benzothiophene-3-carbonitrile.

The following intermediates and compounds can be prepared as described for exemplified examples in an analogous manner.

TABLE 16

| | | tᵣₑₜ | | HPLC |
|---|---|---|---|---|
| | structure | [min] | [M + H]⁺ | method |
| V-1 | 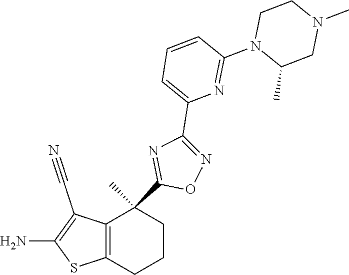 [SMILES: C[C@H]1CN(C)CCN1c1cccc(-c2noc([C@@]3(C)CCCc4sc(N)c(C#N)c43)n2)n1] | 1.39 | 450 | A |
| V-2 | 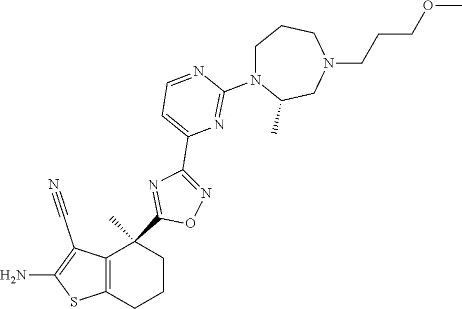 [SMILES: COCCCN1CCCN(c2nccc(-c3noc([C@@]4(C)CCCc5sc(N)c(C#N)c54)n3)n2)[C@@H](C)C1] | 1.40 | 523 | A |
| V-3 | 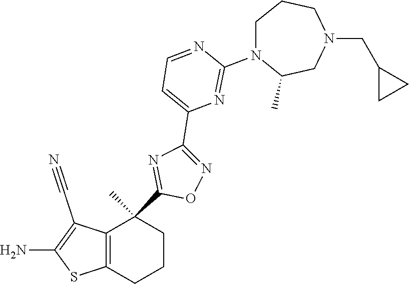 [SMILES: C[C@H]1CN(CC2CC2)CCCN1c1nccc(-c2noc([C@@]3(C)CCCc4sc(N)c(C#N)c43)n2)n1] | 1.54 | 505 | A |
| V-4 | 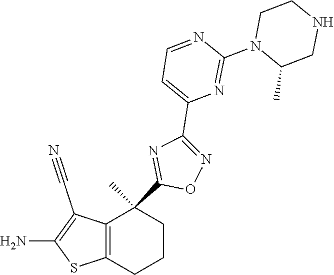 [SMILES: C[C@H]1CNCCN1c1nccc(-c2noc([C@@]3(C)CCCc4sc(N)c(C#N)c43)n2)n1] | 1.21 | 437 | A |
| V-5 | 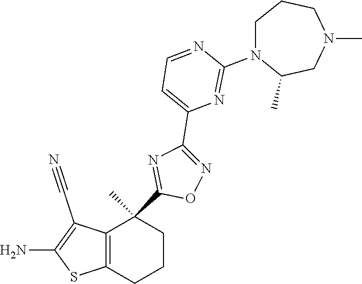 [SMILES: C[C@H]1CN(C)CCCN1c1nccc(-c2noc([C@@]3(C)CCCc4sc(N)c(C#N)c43)n2)n1] | 1.38 | 465 | A |
| V-6 | 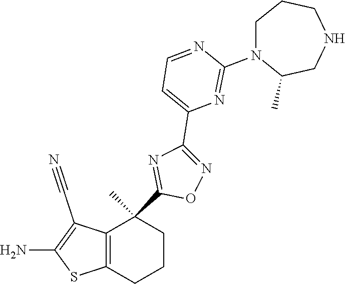 [SMILES: C[C@H]1CNCCCN1c1nccc(-c2noc([C@@]3(C)CCCc4sc(N)c(C#N)c43)n2)n1] | 1.26 | 451 | A |
| V-7 | 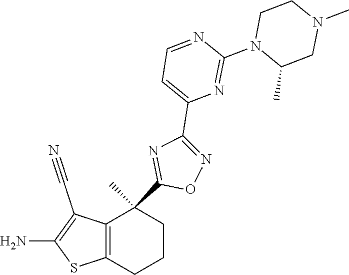 [SMILES: C[C@H]1CN(C)CCN1c1nccc(-c2noc([C@@]3(C)CCCc4sc(N)c(C#N)c43)n2)n1] | 1.39 | 451 | A |
| V-8 | 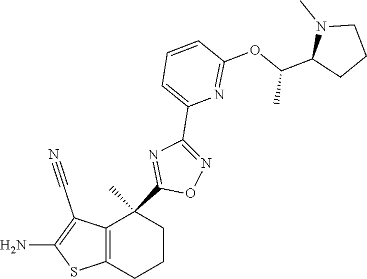 [SMILES: C[C@H](Oc1cccc(-c2noc([C@@]3(C)CCCc4sc(N)c(C#N)c43)n2)n1)[C@@H]1CCCN1C] | 1.48 | 465 | A |
| V-9 | 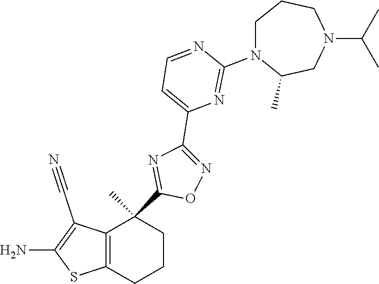 [SMILES: CC(C)N1CCCN(c2nccc(-c3noc([C@@]4(C)CCCc5sc(N)c(C#N)c54)n3)n2)[C@@H](C)C1] | 1.54 | 493 | A |
| V-10 | 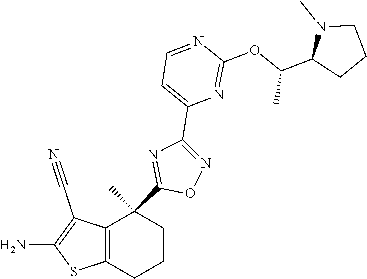 [SMILES: C[C@H](Oc1nccc(-c2noc([C@@]3(C)CCCc4sc(N)c(C#N)c43)n2)n1)[C@@H]1CCCN1C] | 1.33 | 466 | A |
| V-11 | 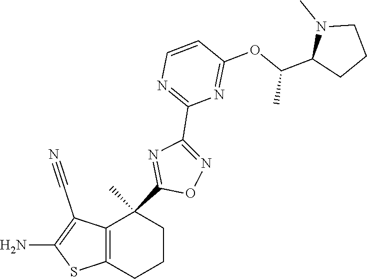 [SMILES: C[C@H](Oc1ccnc(-c2noc([C@@]3(C)CCCc4sc(N)c(C#N)c43)n2)n1)[C@@H]1CCCN1C] | 1.31 | 466 | A |
| V-12 | 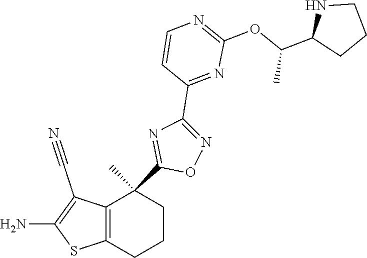 [SMILES: C[C@H](Oc1nccc(-c2noc([C@@]3(C)CCCc4sc(N)c(C#N)c43)n2)n1)[C@@H]1CCCN1] | 1.09 | 452 | A |
| V-13 | 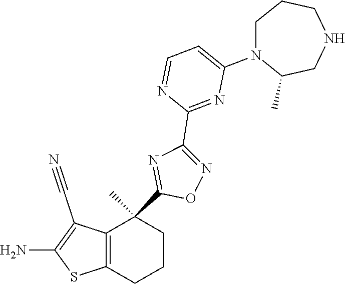 [SMILES: C[C@H]1CNCCCN1c1ccnc(-c2noc([C@@]3(C)CCCc4sc(N)c(C#N)c43)n2)n1] | — | — | — |
| V-14 | 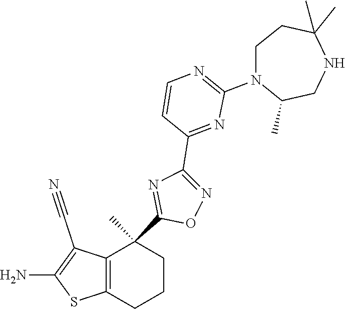 [SMILES: C[C@H]1CNC(C)(C)CCN1c1nccc(-c2noc([C@@]3(C)CCCc4sc(N)c(C#N)c43)n2)n1] | 0.7 | 479 | E |
| V-15 | 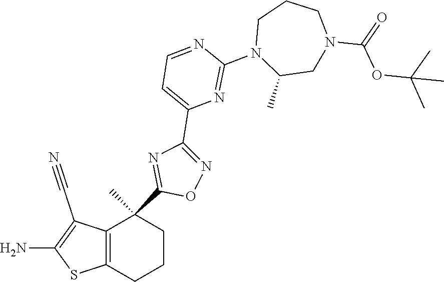 [SMILES: C[C@H]1CN(C(=O)OC(C)(C)C)CCCN1c1nccc(-c2noc([C@@]3(C)CCCc4sc(N)c(C#N)c43)n2)n1] | 1.52 | 551 | A |
| V-16 | 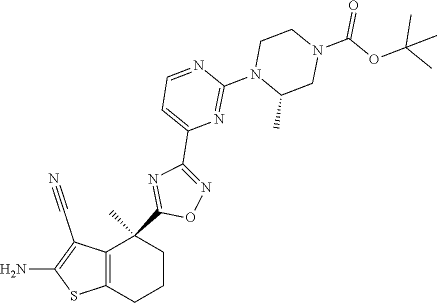 [SMILES: C[C@H]1CN(C(=O)OC(C)(C)C)CCN1c1nccc(-c2noc([C@@]3(C)CCCc4sc(N)c(C#N)c43)n2)n1] | 1.54 | 537 | A |
| V-17 | 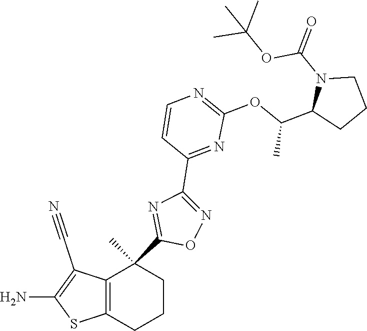 [SMILES: C[C@H](Oc1nccc(-c2noc([C@@]3(C)CCCc4sc(N)c(C#N)c43)n2)n1)[C@@H]1CCCN1C(=O)OC(C)(C)C] | 1.94 | 552 | — |
| V-18 | 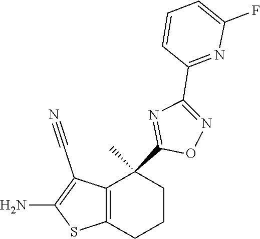 [SMILES: C[C@]1(c2nc(-c3cccc(F)n3)no2)CCCc2sc(N)c(C#N)c21] | 1.26 | 356 | A |
| V-19 | 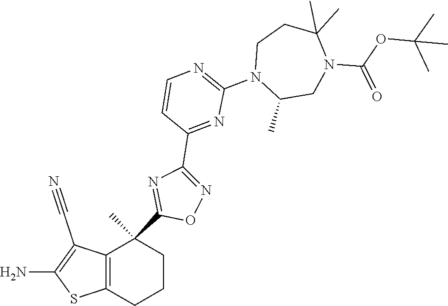 [SMILES: C[C@H]1CN(C(=O)OC(C)(C)C)C(C)(C)CCN1c1nccc(-c2noc([C@@]3(C)CCCc4sc(N)c(C#N)c43)n2)n1] | 2.52 | 579 | H |
| V-20 | 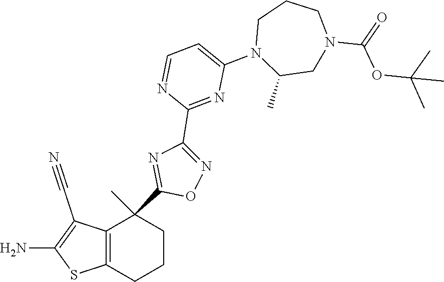 [SMILES: C[C@H]1CN(C(=O)OC(C)(C)C)CCCN1c1ccnc(-c2noc([C@@]3(C)CCCc4sc(N)c(C#N)c43)n2)n1] | 1.36 | 551 | A |

### Experimental Procedure for the Synthesis of K-14a

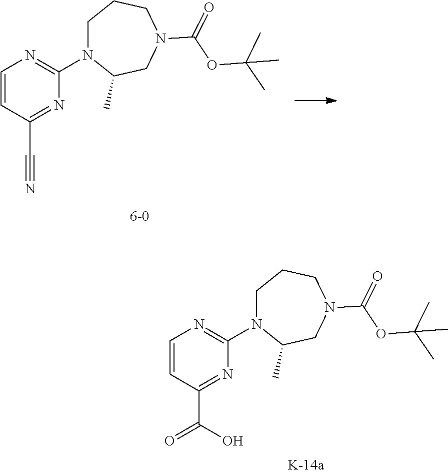 [SMILES: C[C@H]1CN(C(=O)OC(C)(C)C)CCCN1c1nccc(C#N)n1 ; C[C@H]1CN(C(=O)OC(C)(C)C)CCCN1c1nccc(C(=O)O)n1]

To a solution of 6-0 (534 mg, 1.68 mmol, 1.0 eq.) in EtOH (4.0 ml) is added sodium hydroxide (4 M in water, 1.00 ml 40.0 mmol, 2.4 eq.) and stirred under reflux for 1 h. The reaction mixture is allowed to cool to rt and acidified with citric acid (5% in water). Water and EtOAc are added and the phases are separated. The organic layer is washed with water, dried over sodium sulfate, filtered and concentrated under reduced pressure to get the crude product K-14a (Table 17) which is used for the next steps without further purification.

TABLE 17

| | | tᵣₑₜ | | HPLC |
|---|---|---|---|---|
| # | Structure | [min] | [M + H]⁺ | method |
| K-14a | 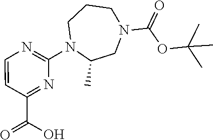 [SMILES: C[C@H]1CN(C(=O)OC(C)(C)C)CCCN1c1nccc(C(=O)O)n1] | 0.77 | 337 | Y |

### Experimental Procedure for the Synthesis of K-15a

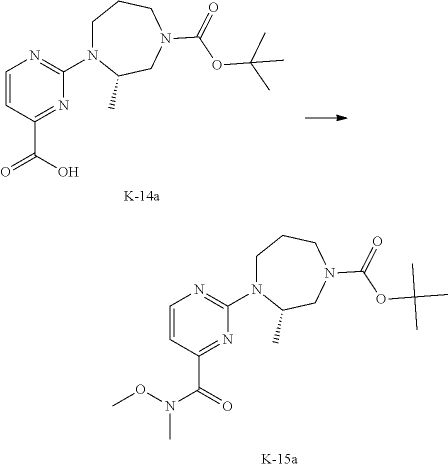

To a stirred solution of K-14a (481.0 mg, 1.43 mmol, 1.0 eq) in dry THF (3.0 mL, 7.5V) are added DIPEA (1.27 mL, 7.15 mmol, 5.0 eq) and HATU (598 mg, 1.57 mmol, 1.1 eq) and stirred at rt for 15 min. N,O-dimethyl hydroxyl amine hydrochloride (278.9 mg, 2.86 mmol, 2.0 eq) is added and stirred at rt for 1 h. After full conversion the reaction mixture is diluted with water and EtOAc and the phases are separated. The organic layer is washed with water, dried over sodium sulfate and concentrated under reduced pressure to get the crude K-15a (Table 18). The crude is purified by chromatography.

TABLE 18

| | | tᵣₑₜ | | HPLC |
|---|---|---|---|---|
| # | Structure | [min] | [M + H]⁺ | method |
| K-15a | 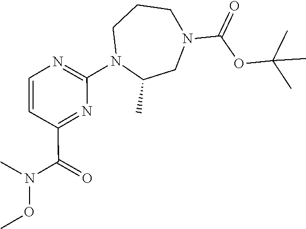 [SMILES: CON(C)C(=O)c1ccnc(N2CCCN(C(=O)OC(C)(C)C)C[C@@H]2C)n1] | 1.22 | 380 | Y |

### Experimental Procedure for the Synthesis of K-16a

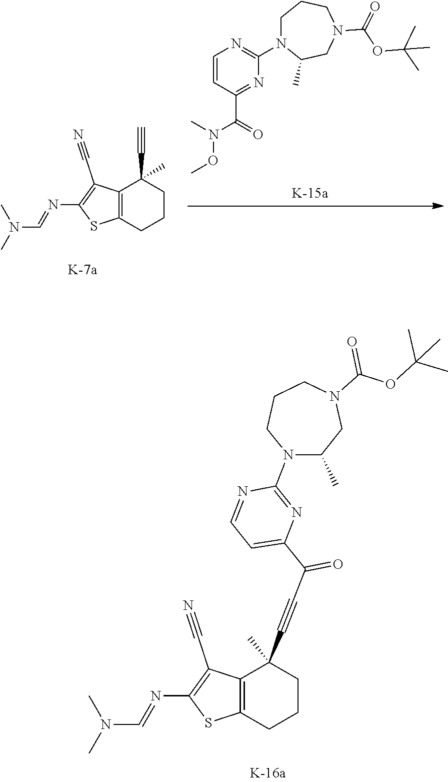

To a solution of K-7a (230 mg, 0.85 mmol, 1.0 eq.) and K-15a (389.0 mg, 1.025 mmol, 1.2eq.) in THF (7 mL) is added LiHMDS (3.39 mL, 3.39 mmol, 4.00 eq., 1 M in THF) at −78° C. dropwise and stirred for 10 min at −78° C. After complete conversion the reaction is allowed to warm to rt, quenched with water and diluted with EtOAc. The phases are separated, and the aqueous layer is extracted three times with EtOAc. The combined organic layer is concentrated under reduced pressure. The residue is taken up in acetonitrile and water and purified by chromatography to give the desired product K-16a (table 19).

TABLE 19

| | | tᵣₑₜ | | HPLC |
|---|---|---|---|---|
| # | Structure | [min] | [M + H]⁺ | method |
| K-16a | 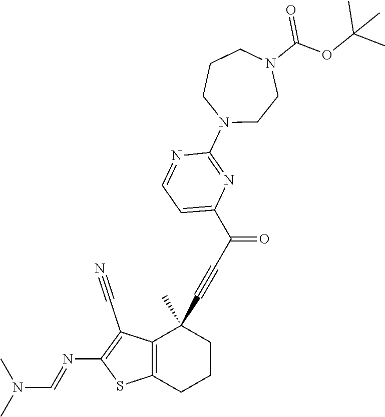 [SMILES: CN(C)/C=N/c1sc2c(c1C#N)[C@@](C)(C#CC(=O)c1ccnc(N3CCCN(C(=O)OC(C)(C)C)CC3)n1)CCC2] | 1.64 | 590 | Y |

### Experimental Procedure for the Synthesis of K-17a

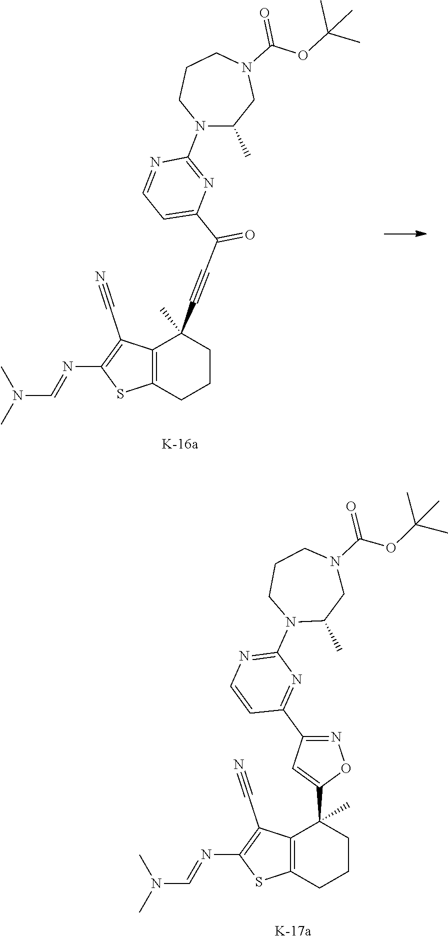

To a stirred solution of K-16a (100 mg, 0.17 mmol, 1.0 eq.) in EtOH (1 mL) is added hydroxylamine hydrochloride (60.1 mg, 0.85 mmol, 5.0 eq.) and stirred at rt for 2 h. After complete conversion the reaction is quenched with saturated sodium hydrogen carbonate solution and stirred at rt for 2 h. The mixture is diluted with acetonitrile and water, filtered and purified by chromatography to give the desired intermediate K-17a (table 20).

TABLE 20

| | | tᵣₑₜ | | HPLC |
|---|---|---|---|---|
| # | Structure | [min] | [M + H]⁺ | method |
| K-17a | 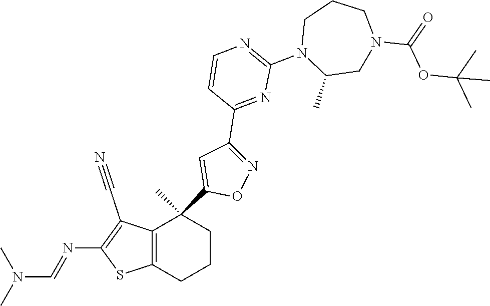 [SMILES: C[C@H]1CN(C(=O)OC(C)(C)C)CCCN1c1nccc(-c2cc([C@@]3(C)CCCc4sc(/N=C/N(C)C)c(C#N)c43)on2)n1] | 1.64 | 605 | Y |

### Experimental Procedure for the Synthesis of K-18a

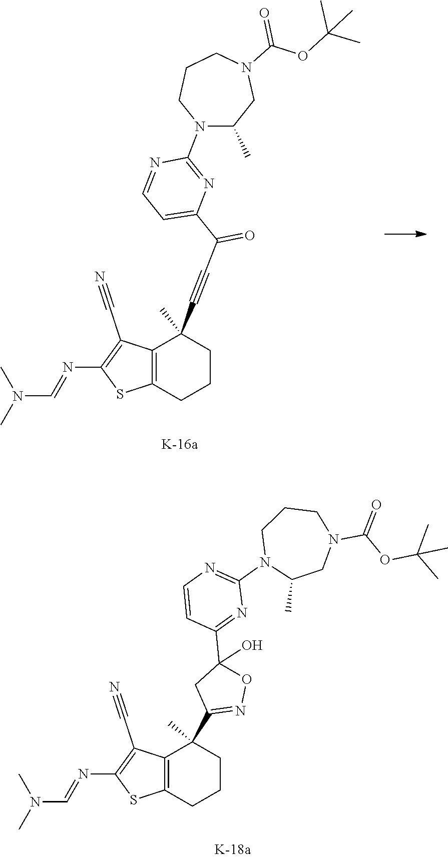

To a stirred solution of K-16a (45 mg, 0.076 mmol, 1.0 eq.) in ACN (0.5 mL) is added hydroxylamine solution (50% in water, 70.14 μL, 1.145 mmol, 15.0 eq.) and the reaction mixture is stirred at rt for 30 min. After completion of conversion the reaction mixture is diluted with acetonitrile and water, filtered and purified by chromatography to give the desired intermediate K-18a (table 21).

TABLE 21

| | | tᵣₑₜ | | HPLC |
|---|---|---|---|---|
| # | Structure | [min] | [M + H]⁺ | method |
| K-18a | 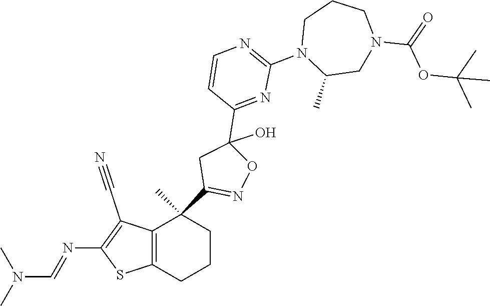 [SMILES: C[C@H]1CN(C(=O)OC(C)(C)C)CCCN1c1nccc(C2(O)CC([C@@]3(C)CCCc4sc(/N=C/N(C)C)c(C#N)c43)=NO2)n1] | 1.48 | 621 | Y |

### Experimental Procedure for the Synthesis of K-17b

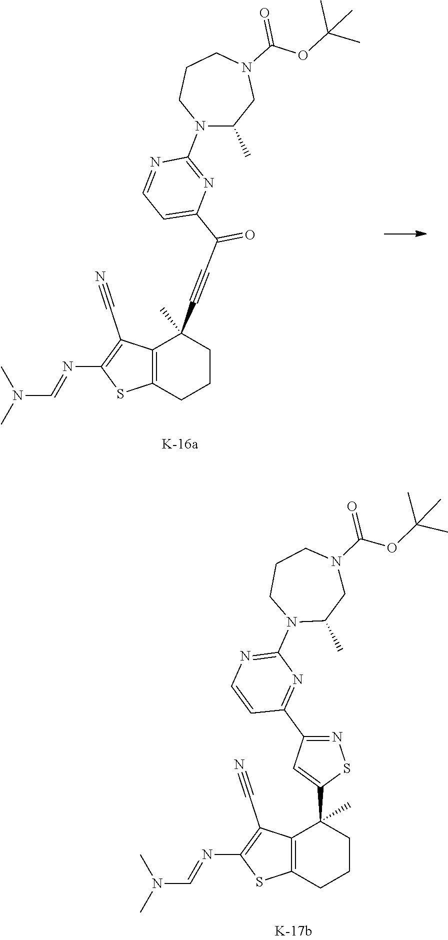

To a solution of K-16a (85.0 mg, 0.144 mmol, 1.0 eq.) in methanol (2 mL) is added hydroxylamine-O-sulfonic acid (32.6 mg, 0.288 mmol, 2.0 eq.). The reaction mixture is stirred for 40 min. Sodium hydrogencarbonate (30.3 mg, 0.360 mmol, 2.5 eq.) and sodium hydrogen sulfide (20.2 mg, 0.360 mmol, 2.5 eq.) are added to the reaction mixture and it is stirred for 18 h. Additional sodium hydrogencarbonate (36.3 mg, 0.432 mmol, 3.0 eq.) and sodium hydrogen sulfide (24.2 mg, 0.432 mmol, 3.0 eq.) are added and the reaction mixture is stirred at 60° C. for 2 hours. The reaction mixture is diluted with DCM and saturated aqueous sodium hydrogencarbonate solution. The phases are separated and the aqueous layer is extracted with DCM (5 times). The organic layers are combined and the solvent is removed under reduced pressure. The crude product is purified by chromatography to give intermediate K-17b (table 22).

TABLE 22

| | | tᵣₑₜ | | HPLC |
|---|---|---|---|---|
| # | Structure | [min] | [M + H]⁺ | method |
| K-17b | 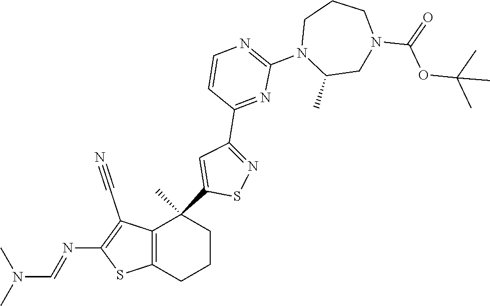 [SMILES: C[C@H]1CN(C(=O)OC(C)(C)C)CCCN1c1nccc(-c2cc([C@@]3(C)CCCc4sc(/N=C/N(C)C)c(C#N)c43)sn2)n1] | 1.69 | 581 | Y |

### Experimental Procedure for the Synthesis of V-21

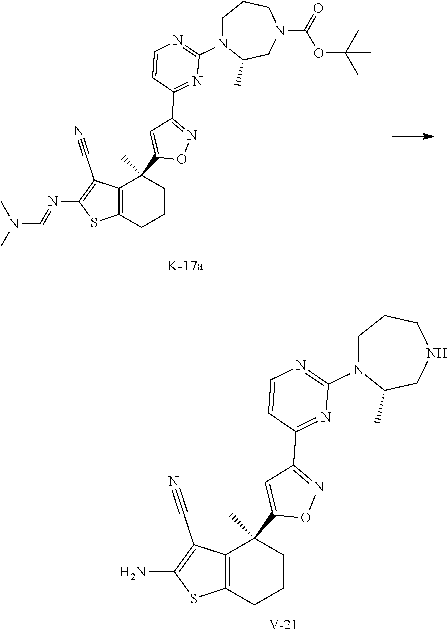

To a stirred solution of K-17a (114.0 mg, 0.19 mmol, 1.0 eq.) in THF (1 mL) is added HCl (2 M in water, 0.94 mL, 1.89 mmol, 10.0 eq.) at rt. The reaction mixture is stirred at 65° C. for 2 h. After complete conversion the reaction mixture is concentrated under reduced pressure, diluted with water and acetonitrile and brought to an alkaline pH by addition of 2 M NaOH. It is purified by reversed phase chromatography using basic conditions to yield V-21.

The following compounds (table 23) are available in an analogous manner using different intermediates K-17. The crude product K-17 is purified by chromatography if necessary.

TABLE 23

| | | tᵣₑₜ | | HPLC |
|---|---|---|---|---|
| # | structure | [min] | [M + H]⁺ | method |
| V-21 | 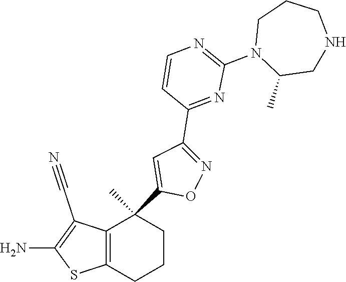 [SMILES: C[C@H]1CNCCCN1c1nccc(-c2cc([C@@]3(C)CCCc4sc(N)c(C#N)c43)on2)n1] | 1.26 | 450 | Y |
| V-22 | 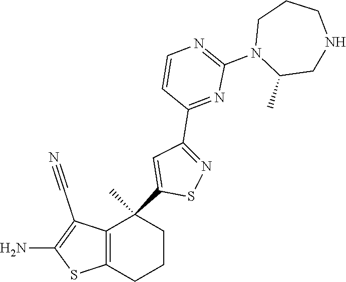 [SMILES: C[C@H]1CNCCCN1c1nccc(-c2cc([C@@]3(C)CCCc4sc(N)c(C#N)c43)sn2)n1] | 1.31 | 466 | Y |

### Experimental Procedure for the Synthesis of V-23

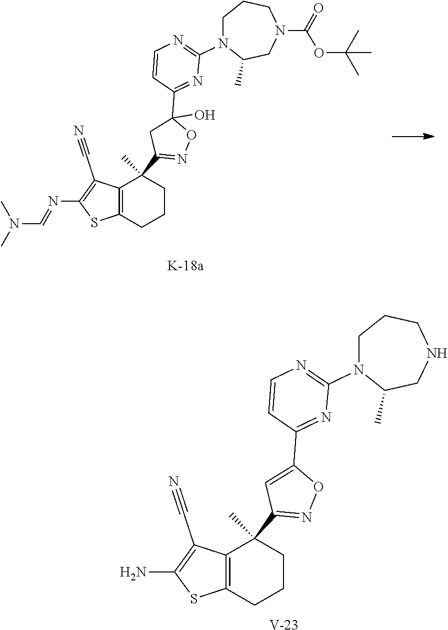

To a stirred solution of K-18a (40.0 mg, 0.06 mmol, 1.0 eq.) in THF (0.5 mL) is added HCl (2 M in water, 0.32 mL, 0.64 mmol, 10.0 eq.) at rt. The reaction mixture is stirred at 65° C. for 3 h. After complete conversion the reaction mixture is concentrated under reduced pressure, diluted with water and acetonitrile and basified by addition of NaOH (2 M in water). The mixture is purified by chromatography to yield V-23 (table 24).

TABLE 24

| | | tᵣₑₜ | | HPLC |
|---|---|---|---|---|
| # | structure | [min] | [M + H]⁺ | method |
| V-23 | 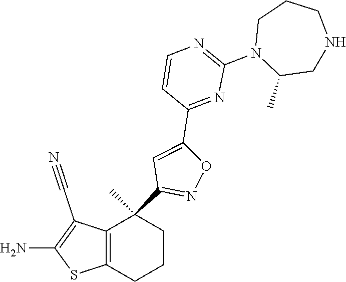 [SMILES: C[C@H]1CNCCCN1c1nccc(-c2cc([C@@]3(C)CCCc4sc(N)c(C#N)c43)no2)n1] | 1.26 | 450 | Y |

The following compounds of formula (V) according to the invention can also be obtained analogously following the procedures as described herein and using the corresponding building blocks:

TABLE 25

| | | tᵣₑₜ | | HPLC |
|---|---|---|---|---|
| # | structure | [min] | [M + H]⁺ | method |
| V-24 | 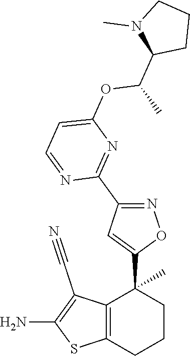 [SMILES: C[C@H](Oc1ccnc(-c2cc([C@@]3(C)CCCc4sc(N)c(C#N)c43)on2)n1)[C@@H]1CCCN1C] | 465 | 1.36 | A |
| V-25 | 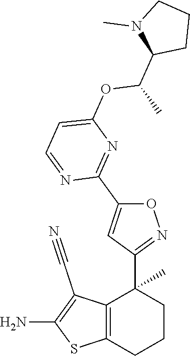 [SMILES: C[C@H](Oc1ccnc(-c2cc([C@@]3(C)CCCc4sc(N)c(C#N)c43)no2)n1)[C@@H]1CCCN1C] | 465 | 1.38 | A |
| V-26 | 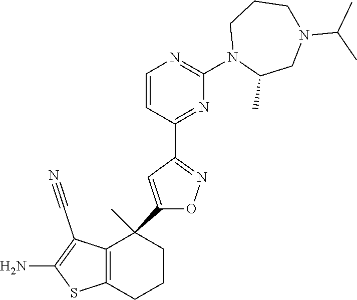 [SMILES: CC(C)N1CCCN(c2nccc(-c3cc([C@@]4(C)CCCc5sc(N)c(C#N)c54)on3)n2)[C@@H](C)C1] | 492 | 1.55 | A |
| V-27 | 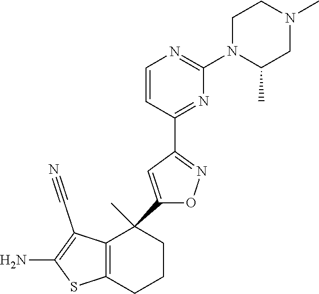 [SMILES: C[C@H]1CN(C)CCN1c1nccc(-c2cc([C@@]3(C)CCCc4sc(N)c(C#N)c43)on2)n1] | 450 | 1.43 | A |

The following Examples describe the biological activity of the compounds according to the invention, without restricting the invention to these Examples.

### KRAS::SOS1 AlphaScreen Binding Assay

This assay can be used to examine the potency with which compounds according to the invention binding to (mutated) KRAS inhibit the protein-protein interaction between SOS1 and (mutated) KRAS e.g., KRAS WT, KRAS G12C, KRAS G12D, KRAS G12V or KRAS G13D. This inhibits the GEF functionality of SOS1 and locks the corresponding (mutated) KRAS protein in its inactive, GOP-bound state. Low IC₅₀ values in this assay setting are indicative of strong inhibition of protein-protein interaction between SOS1 and KRAS:

### Description of the Assay:

These assays measure the inhibitory effect of compounds on KRAS mutant protein-protein interactions using the Alpha Screen technology by Perkin Elmer.

The following (mutant) enzyme forms of KRAS and interacting proteins are used in these assays at the given concentrations:
KRAS (G12D) 1-169, N-terminal 6His-tag, C-terminal avi-tag (Xtal BioStructures, Inc.); final assay concentration 10 nM and SOS1 564-1049, N-terminal 229 GST-tag, TEV cleavage site (Viva Biotech Ltd); final assay concentration 5 nM;
KRAS (G12C) 1-169, N-terminal 6His-tag for purification, cleaved off, C-terminal avi-tag, biotinylated, mutations: C51S, C80L, C118S (in house); final assay concentration 7.5 nM and SOS1 564-1049, N-terminal 229 GST-tag, TEV cleavage site (Viva Biotech Ltd); final assay concentration 5 nM;
KRAS (G12V) 1-169, N-terminal 6His-tag for purification, cleaved off, C-terminal avi-tag, biotinylated, TEV cleavage site, mutation: C118S, GDP loaded (in house); final assay concentration 10 nM and SOS1 564-1049, N-terminal 229 GST-tag, TEV cleavage site (Viva Biotech Ltd); final assay concentration 10 nM;
KRAS (G13D) 1-169, N-terminal 6His-tag for purification, cleaved off, C-terminal avi-tag, biotinylated, TEV cleavage site, mutation: C118S, GDP loaded (in house); final assay concentration 10 nM and SOS1 564-1049, N-terminal 229 GST-tag, TEV cleavage site (Viva Biotech Ltd); final assay concentration 10 nM;
KRAS (WT) 1-169, N-terminal 6His-tag for purification, cleaved off, C-terminal avi-tag, biotinylated, TEV cleavage site, mutation: C118S, GDP loaded (in house); final assay concentration 10 nM and SOS1 564-1049, N-terminal 229 GST-tag, TEV cleavage site (Viva Biotech Ltd); final assay concentration 10 nM.

Test compounds dissolved in DMSO are dispensed onto assay plates (Proxiplate 384 PLUS, white, PerkinElmer; 6008289) using an Access Labcyte Workstation with the Labcyte Echo 55x. For the chosen highest assay concentration of 100 μM, 150 nL of compound solution are transferred from a 10 mM DMSO compound stock solution. A series of eleven fivefold dilutions per compound are transferred to the assay plate, compound dilutions are tested in duplicates. DMSO are added as backfill to a total volume of 150 nL.

The assays run on a fully automated robotic system in a darkened room below 100 Lux. To 150 nl of compound dilution 10 μl of a mix including KRAS mutant protein, SOS1 (final assay concentrations see above) and GDP nucleotide (Sigma G7127; final assay concentration 10 μM) in assay buffer (1×PBS, 0.1% BSA, 0.05% Tween 20) are added into columns 1-24. After 30 minutes incubation time 5 μl of Alpha Screen bead mix in assay buffer are added into columns 1-23. Bead mix consists of AlphaLISA Glutathione Acceptor Beads (PerkinElmer, Cat No AL109) and AlphaScreen Streptavidin Donor Beads (PerkinElmer Cat No 6760002) in assay buffer at a final assay concentration of 10 μg/ml each.

Plates are kept at room temperature in a darkened incubator. After an additional 60 minutes incubation time the signal is measured in a PerkinElmer Envision HTS Multilabel Reader using the AlphaScreen specs from PerkinElmer.

Each plate contains up to 16 wells of a negative control depending on the dilution procedure (platewise or serial) (DMSO instead of test compound; with KRAS mutant::SOS1 GDP mix and bead mix; column 23) and 16 wells of a positive control (DMSO instead of test compound; with KRAS mutant::SOS1 GDP mix w/o bead mix; column 24).

As internal control known inhibitors of KRAS mutant::SOS1 interaction can be measured on each compound plate.

IC50 values are calculated and analyzed with Boehringer Ingelheim's MEGALAB IC50 application using a 4 parametric logistic model.

Tables of example compounds disclosed herein contain IC₅₀ values determined using the above assays (see Table 26).

TABLE 26

| | KRAS | KRAS | KRAS | KRAS | KRAS |
|---|---|---|---|---|---|
| | G12D | G12C | G12V | G13D | WT |
| Ex | IC₅₀ | IC₅₀ | IC₅₀ | IC₅₀ | IC₅₀ |
| # | (nM) | (nM) | (nM) | (nM) | (nM) |
| V-1 | 26 | 31 | 52 | 928 | 125 |
| V-2 | 3 | 6 | 6 | | |
| V-3 | 3 | 6 | 5 | | |
| V-4 | 15 | 23 | | | |
| V-5 | 7 | 8 | | | |
| V-6 | 12 | 15 | | | |
| V-7 | 16 | 22 | | | |
| V-8 | 7 | 9 | 9 | 176 | 28 |
| V-9 | 5 | 5 | | | |
| V-10 | 5 | 7 | | | |
| V-11 | 3 | 5 | 8 | | 13 |
| V-24 | 2 | 4 | | | |
| V-25 | 9 | 14 | 11 | | |
| V-26 | 2 | 7 | | | |
| V-27 | 8 | 24 | 18 | | |

### Ba/F3 Cell Model Generation and Proliferation Assay

Ba/F3 cells are ordered from DSMZ (ACC300, Lot17) and grown in RPMI-1640 (ATCC 30-2001)+10% FCS+10 ng/mL IL-3 at 37° C. in 5% CO₂ atmosphere. Plasmids containing KRASG12 mutants (i.e. G12D, G12C, G12V) are obtained from GeneScript. To generate KRASG12-dependent Ba/F3 models, Ba/F3 cells are transduced with retroviruses containing vectors that harbor KRASG12 isoforms. Platinum-E cells (Cell Biolabs) are used for retrovirus packaging. Retrovirus is added to Ba/F3 cells. To ensure infection, 4 μg/mL polybrene is added and cells are spinfected. Infection efficiency is confirmed by measuring GFP-positive cells using a cell analyzer. Cells with an infection efficiency of 10% to 20% are further cultivated and puromycin selection with 1 μg/mL is initiated. As a control, parental Ba/F3 cells are used to show selection status. Selection is considered successful when parental Ba/F3 cells cultures died. To evaluate the transforming potential of KRASG12 mutations, the growth medium is no longer supplemented with IL-3. Ba/F3 cells harboring the empty vector are used as a control. Approximately ten days before conducting the experiments, puromycin is left out.

For proliferation assays, Ba/F3 cells are seeded into 384-well plates at 1.5×10³ cells/60 μL in growth media (RPMI-1640+10% FCS). Compounds are added using an Access Labcyte Workstation with a Labcyte Echo 550 or 555 acoustic dispenser. All treatments are performed in technical duplicates. Treated cells are incubated for 72 h at 37° C. with 5% CO₂. AlamarBlue™ (ThermoFisher), a viability stain, is added and fluorescence measured in the PerkinElmer Envision HTS Multilabel Reader. The raw data are imported into and analyzed with the Boehringer Ingelheim proprietary software MegaLab (curve fitting based on the program PRISM, GraphPad Inc.).

IC₅₀ values of representative compounds according to the invention measured with this assay are presented in table 27.

TABLE 27

| | KRAS G12D | KRAS G12C | KRAS G12V |
|---|---|---|---|
| Ex # | Ba/F3 IC₅₀ (nM) | Ba/F3 IC₅₀ (nM) | Ba/F3 IC₅₀ (nM) |
| V-1 | 961 | 1274 | 1436 |
| V-2 | 256 | 371 | 917 |
| V-3 | 214 | 409 | 516 |
| V-4 | 823 | 831 | 1082 |
| V-5 | 293 | 493 | 640 |
| V-6 | 801 | 811 | 1074 |
| V-7 | 1089 | 1235 | 1678 |
| V-8 | 390 | 481 | 443 |
| V-9 | 51 | 64 | 76 |
| V-10 | 214 | 291 | 361 |
| V-11 | 93 | 94 | 113 |
| V-24 | 108 | 105 | |
| V-25 | 961 | 940 | 6064 |
| V-26 | 79 | 95 | |
| V-27 | 525 | 1434 | 1695 |

### ERK Phosphorylation Assay

ERK phosphorylation assays are used to examine the potency with which compounds inhibit the KRAS G12C-mediated signal transduction in a KRAS G12C mutant human cancer cell line in vitro. This demonstrates the molecular mode of action of compounds according to the invention by interfering with the RAS G12C protein signal transduction cascade. Low IC₅₀ values in this assay setting are indicative of high potency of the compounds according to the invention. It is observed that compounds according to the invention demonstrate an inhibitory effect on ERK phosphorylation in a KRAS G12C mutant human cancer cell line, thus confirming the molecular mode of action of the compounds on RAS G12C protein signal transduction.

ERK phosphorylation assays are performed using the following human cell lines: NCI-H358 (ATCC (ATCC CRL-5807): human lung cancer with a KRAS G12C mutation (→assay 1) and NCI-H358_Cas9_SOS2, i.e. the same cell line, in which SOS2 is knocked (→assay 2). Vectors containing the designed DNA sequences for the production of gRNA for SOS2 protein knock-out are obtained from Sigma-Aldrich. To generate the NCI-H358 SOS2 knock-out cell line, NCI-H358 cells expressing Cas9 endonuclease are transfected with XtremeGene9 reagent and the correspondent plasmids. Transfection efficiency is confirmed by measuring GFP-positive cells using a cell analyzer. GFP positive cells are collected and further expanded. These GFP-positive cell pools are single-cell diluted and SOS2 knock-out clones are identified via Western-blot and genomic DNA sequencing analysis.

### Materials Used for the Assay:

RPMI-1640 Medium (ATCC® 30-2001™)
Fetal Bovine Serum (FBS) from HyClone (SH30071.03)
Non-essential amino acids from Thermo Fischer Scientific (11140035)
Pyruvate from Thermo Fischer Scientific (11360039)
Glutamax from Thermo Fischer Scientific (35050061)
384 plates from Greiner Bio-One (781182)
Proxiplate™ 384 from PerkinElmer Inc. (6008280)
AlphaLISA SureFire Ultra p-ERK1/2 (Thr202/Tyr204) Assay Kit (ALSU-PERK-A500)
EGF from Sigma (E4127)
Acceptor Mix: Protein A Acceptor Beads from PerkinElmer (6760137M)
Donor Mix: AlphaScreen Streptavidin-coated Donor Beads from PerkinElmer (6760002)
Trametinib
Staurosporine from Sigma Aldrich (S6942)

### Assay Setup:

Cells are seeded at 40,000 cells per well in/60 μL of RPMI with 10% FBS, non-essential amino acids, pyruvate and glutamax in Greiner TC 384 plates. The cells are incubated for 1 h at room temperature and then incubated overnight in an incubator at 37° C. and 5% CO₂ in a humidified atmosphere. 60 nL compound solution (10 mM DMSO stock solution) is then added using a Labcyte Echo 550 device. After a 1 h incubation in the aforementioned incubator the medium is removed after centrifugation and the cells lysed by addition of 20 μL of 1.6-fold lysis buffer from the AlphaLISA SureFire Ultra pERK1/2 (Thr202/Tyr204) Assay Kit with added protease inhibitors, 100 nM trametinib+100 nM staurosporine. After 20 min of incubation at room temperature with shaking, 6 μL of each lysate sample is transferred to a 384-well Proxiplate and analyzed for pERK (Thr202/Tyr204) with the AlphaLISA SureFire Ultra pERK1/2 (Thr202/Tyr204) Assay Kit. 3 μL Acceptor Mix and 3 μL Donor Mix are added under subdued light and incubated for 2 h at room temperature in the dark, before the signal is measured on a PerkinElmer Envision HTS Multilabel Reader. The raw data are imported into and analyzed with the Boehringer Ingelheim proprietary software MegaLab (curve fitting based on the program PRISM, GraphPad Inc.).

Analogously the described assay (pERK reduction; SureFire) can be performed on additional cell lines, carrying various KRAS mutations or KRAS wildtype, allowing the measurement and determination of the activity of compounds on various additional KRAS allels in a cellular background.

IC₅₀ values of representative compounds according to the invention measured with this assay are presented in table 28 (IC₅₀s from assay 2 are marked with *, all others are from assay 1).

TABLE 28

| Ex # | IC₅₀ H358 pERK [nM] |
|---|---|
| V-1 | 252 |
| V-2 | 23 |
| V-3 | 24 |
| V-4 | 130 |
| V-5 | 38 |
| V-6 | 75 |
| V-7 | 137 |
| V-8 | 55 |
| V-9 | 12 |
| V-10 | 12 |
| V-11 | 7 |
| V-24 | 19 |
| V-25 | 100 |

Additional Proliferation Assays with Mutant Cancer Cell Lines

### NCI-H358 CTG Proliferation Assay (120 h) (NSCLC, G12C)

NCI-H358 cells (ATCC No. CRL-5807) are dispensed into white bottom opaque 96 well plates (Perkin Elmer cat no. 5680) at a density of 2000 cells per well in 100 μL RPMI-1640 ATCC-Formulation (Gibco #A10491)+10% FCS (fetal calf serum) (assay 1) or into black 384-well plates, flat and clear bottom (Greiner, PNr. 781091) at a density of 200 cells per well in 60 μl RPMI-1640 ATCC-Formulation (Gibco #A10491)+10% FCS (fetal calf serum) (assay 2). Cells are incubated overnight at 37° C. in a humidified tissue culture incubator at 5% CO₂. Compounds (10 mM stock in DMSO) are added at logarithmic dose series using the HP Digital Dispenser D300 (Tecan) (assay 1) or the ECHO acoustic liquid handler system (Beckman Coulter) (assay 2), normalizing for added DMSO and including DMSO controls. For the T0 time point measurement, untreated cells are analyzed at the time of compound addition. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### NCI-H2122 CTG Proliferation Assay (120 h) (NSCLC, G12C)

The CTG assay is designed to measure quantitatively the proliferation of NCI-H2122 cells (ATCC CRL-5985), using the CellTiter Glow Assay Kit (Promega G7571). Cells are grown in RPMI medium (ATCC) supplemented with Fetal Calf Serum (Life Technologies, Gibco BRL, Cat. No. 10270-106). On “day 0” 200 NCI-H2122 cells are seeded in 60 μL RPMI ATCC+10% FCS+ Penstrep in a black 384-well plate, flat and clear bottom (Greiner, PNr. 781091). Cells are then incubated in the plates at 37° C. in a CO₂ incubator overnight. On day 1, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### AsPC-1 CTG Proliferation Assay (120 h) (Pancreatic Cancer, G12D)

The CTG assay is designed to measure quantitatively the proliferation of AsPC-1 cells (ATCC CRL-5985), using the CellTiter Glow Assay Kit (Promega G7571). Cells are grown in RPMI medium (ATCC) supplemented with Fetal Calf Serum (Life Technologies, Gibco BRL, Cat. No. 10270-106). On “day 0” 2000 AsPC-1 cells are seeded in 60 μL RPMI ATCC+10% FCS+Penstrep in a 384-well plate, flat and clear bottom (Greiner, PNr. 781091). Cells are then incubated in the plates at 37° C. in a CO₂ incubator overnight. On day 1, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### GP2D Proliferation Assay (120 h) (Colorectal Cancer, G12D)

GP2D cells (ATCC No. CRL-5807) are dispensed into white 384-well plates, flat and white bottom(Perkin Elmer, 6007680) at a density of 500 cells per well in 40 μl DMEM (Sigma, D6429)+1×GlutaMAX (Gibco, 35050038)+10% FCS (fetal calf serum). Cells are incubated overnight at 37° C. in a humidified tissue culture incubator at 5% CO₂. Compounds (10 mM stock in DMSO) are added at logarithmic dose series using the HP Digital Dispenser D300 (Tecan), including DMSO controls and normalizing for added DMSO. For the T0 time point measurement, untreated cells are analyzed at the time of compound addition. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### SAS CTG Proliferation Assay (120 h) (HNSCC, wt Amplified)

SAS cells (JCRB0260) are dispensed into 384-well plates, flat and clear bottom (Greiner, PNr. 781091) at a density of 300 cells per well in 60 μL DMEM:F12 (Gibco 31330-038)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### SK-CO-1 CTG Proliferation Assay (120 h) (CRC, G12V)

SK-CO-1 cells (ATCC HTB-39) are dispensed into 384-well plates, flat and clear bottom (Greiner, PNr. 781091) at a density of 500 cells per well in 60 μL EMEM (Sigma M5650)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### LOVO CTG Proliferation Assay (120 h) (CRC, G13D)

LOVO cells (ATCC CCL-229) are dispensed into 384-well plates, flat and clear bottom (Greiner, PNr. 781091) at a density of 1000 cells per well in 60 μL DMEM (Sigma D6429)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

IC₅₀ values of representative compounds according to the invention measured with these assays in the indicated cell lines are presented in table 29.

TABLE 29

| | H358 | H358 | | | | SK- | |
|---|---|---|---|---|---|---|---|
| | (assay 1) | (assay 2) | AsPC-1 | GP2D | SAS | CO-1 | LOVO |
| | IC₅₀ | IC₅₀ | IC₅₀ | IC₅₀ | IC₅₀ | IC₅₀ | IC₅₀ |
| Ex # | (nM) | (nM) | (nM) | (nM) | (nM) | (nM) | (nM) |
| V-1 | | 937 | 7243 | | 8024 | 4803 | 8467 |
| V-8 | | 230 | 3946 | | 3100 | 6654 | 1431 |
| V-9 | 698 | 1192 | | | | | |
| V-10 | 5408 | | | | | | |
| V-11 | | 244 | 3698 | 152 | 3330 | 737 | 193 |

### Metabolic (Microsomal) Stability Assay

The metabolic degradation of the test compound is assayed at 37° C. with pooled liver microsomes (mouse (MLM), rat (RLM) or human (HLM)). The final incubation volume of 48 μL per time point contains TRIS buffer (pH 7.5; 0.1 M), magnesium chloride (6.5 mM), microsomal protein (0.5 mg/mL for mouse/rat, 1 mg/mL for human specimens) and the test compound at a final concentration of 1 μM. Following a short preincubation period at 37° C., the reactions are initiated by addition of 12 μL beta-nicotinamide adenine dinucleotide phosphate, reduced form (NADPH, 10 mM) and terminated by transferring an aliquot into solvent after different time points (0, 5, 15, 30, 60 min). Additionally, the NADPH-independent degradation is monitored in incubations without NADPH, terminated at the last time point by addition of acetonitrile. The quenched incubations are pelleted by centrifugation (4,000 rpm, 15 min). An aliquot of the supernatant is assayed by LC-MS/MS to quantify the concentration of parent compound in the individual samples.

In vitro intrinsic clearance (CL_{int, in vitro}) is calculated from the time course of the disappearance of the test drug during the microsomal incubation. Each plot is fitted to the first-order elimination rate constant as C(t)=C₀*exp(−ke*t), where C(t) and C₀ are the concentration of unchanged test drug at incubation time t and that at preincubation and ke is the disappearance rate constant of the unchanged drug. Subsequently, CL_{int, in vitro} (μL min⁻¹·amount protein) values are converted to predicted CL_{int, in vivo} (mL min⁻¹·kg⁻¹) from incubation parameters according to the equation CL_{int, in vivo}=CL_{int, in vitro}×(incubation volume (ml)/amount protein (mg))×(amount protein (mg)/g liver tissue)×(liver weight/body wt.). For better across species comparison the predicted clearance is expressed as percent of the liver blood flow [% QH](mL min⁻¹·kg⁻¹) in the individual species. In general, high stability (corresponding to low % QH) of the compounds across species is desired.

Table 30 shows metabolic stability data obtained with the disclosed assay in HLM for a selection of compounds according to the invention.

TABLE 30

| | HLM QH |
|---|---|
| Ex # | [%] |
| V-1 | 93 |
| V-2 | >95 |
| V-3 | >95 |
| V-4 | 83 |
| V-5 | 94 |
| V-6 | 77 |
| V-7 | 95 |
| V-8 | >88 |
| V-9 | >88 |
| V-10 | 87 |
| V-11 | 81 |
| V-27 | >88 |

### Plasma Protein Binding Assay (PPB)

Binding of test compounds to plasma was determined using equilibrium dialysis (ED) and quantitative mass spectrometry interfaced with liquid chromatography (LC-MS). In brief, ED was performed with dialysis devices consisting of two chambers separated by a semipermeable membrane with a molecular weight cut-off of 5-10 kg/mol. One chamber was filled with 10% FCS in PBS containing 1-10 μmol/L test compound and the other chamber was filled with phosphate-buffer saline (PBS) with or without dextran. The dialysis chamber was incubated for 3-5 hours at 37° C. After incubation, protein was precipitated from aliquots of each chamber and the concentration of test compound in the supernatant of the plasma-containing compartment (cₛₑᵣᵤₘ) and of the buffer-containing compartment (c_{buffer}) was determined by LC-MS. The fraction of unbound test compound (not bound to plasma) (fᵤ) was calculated according to the following equation:

$${f}_{u} [ \% ] = \frac{{C}_{buffer}}{{C}_{plasma}} \times 100$$

Table 31 shows metabolic stability data obtained with the disclosed assay for a selection of compounds according to the invention.

TABLE 31

| | PPB 10% FCS |
|---|---|
| Ex # | (% fu) |
| V-1 | 21 |
| V-8 | 51 |
| V-11 | 61 |

### Mechanism Based Inhibition of CYP3A4 Assay (MBI 3A4):

The time dependent inhibition towards CYP3A4 is assayed in human liver microsomes (0.02 mg/mL) with midazolam (15 μM) as a substrate. The test compounds and water control (wells w/o test compound) are preincubated in presence of NADPH (1 mM) with human liver microsomes (0.2 mg/mL) at a concentration of 25 uM for 0 min and 30 min. After preincubation, the incubate is diluted 1:10 and the substrate midazolam is added for the main incubation (15 min). The main incubation is quenched with acetonitrile and the formation of hydroxy-midazolam is quantified via LC/MS-MS. The formation of hydroxy-midazolam from the 30 min preincubation relative to the formation from the 0 min preincubation is used as a readout. Values of less than 100% mean that the substrate midazolam is metabolized to a lower extent upon 30 min preincubation compared to 0 min preincubation. In general low effects upon 30 min preincubation are desired (corresponding to values close to 100%/not different to the values determined with water control).

Table 32 shows data obtained with the disclosed assay for a selection of compounds according to the invention.

TABLE 32

| | MBI 3A4 |
|---|---|
| Ex # | [%] |
| V-1 | 65 |
| V-4 | 75 |
| V-5 | 55 |
| V-7 | 57 |
| V-9 | 49 |
| V-10 | 65 |
| V-27 | 71 |

### Solubility Measurement (DMSO Solution Precipitation Method)

A 10 mM DMSO stock solution of a test compound is used to determine its aqueous solubility. The DMSO solution is diluted with an aqueous medium (McIlvaine buffer with pH=4.5 or 6.8) to a final concentration of 250 μM. After 24 h of shaking at ambient temperature a potentially formed precipitate is removed by filtration. The concentration of the test compound in the filtrate is determined by LC-UV methods by calibrating the signal to the signal of a reference solution with complete dissolution of the test compound in acetonitrile/water (1:1) with known concentration.

Table 33 shows data obtained with the disclosed assay for a selection of compounds according to the invention.

TABLE 33

| | solubility | solubility |
|---|---|---|
| | [μg/ml] | [μg/ml] |
| Ex # | pH 4.5 | pH 6.8 |
| V-1 | >110 | 76 |
| V-2 | >130 | 100 |
| V-3 | >100 | 82 |
| V-4 | >97 | >91 |
| V-5 | >100 | 92 |
| V-6 | 88 | 85 |
| V-8 | >96 | >95 |
| V-9 | >110 | >110 |
| V-10 | >110 | >110 |
| V-11 | >116 | >116 |
| V-24 | >110 | >110 |
| V-25 | >110 | >100 |
| V-26 | >100 | 95 |
| V-27 | >93 | 18 |

### Caco-2 Assay

The assay provides information on the potential of a compound to pass the cell membrane, on the extent of oral absorption as well as on whether the compound is actively transported by uptake and/or efflux transporters. Permeability measurements across polarized, confluent Caco-2 cell monolayers grown on permeable filter supports (Corning, catalog #3391) are used. 10 μM test compound solution in assay buffer (128.13 mM NaCl, 5.36 mM KCl, 1 mM MgSO₄, 1.8 mM CaCl₂, 4.17 mM NaHCO₃, 1.19 mM Na₂HPO₄, 0.41 mM NaH₂PO₄, 15 mM 2-[4-(2-hydroxyethyl)piperazin-1-yl]ethanesulfonic acid (HEPES), 20 mM glucose, pH 7.4) was added to the donor compartment of the cell chamber containing a monolayer of Caco-2 cells in between the donor and the receiver compartment. The receiver and donor compartments contain 0.25% bovine serum albumine (BSA) in assay buffer. Passive diffusion and/or active transport of compounds across the monolayer is measured in both apical to basolateral (a-b) and basolateral to apical (b-a) direction. a-b permeability (PappAB) represents drug absorption from the intestine into the blood and b-a permeability (PappBA) drug secretion from the blood back into the intestine via both passive permeability as well as active transport mechanisms mediated by efflux and uptake transporters that are expressed on the Caco-2 cells. After a pre-incubation of 25-30 min at 37° C., at predefined time points (0, 30, 60 and 90 min), samples were taken from the receiver and donor compartment, respectively. Concentrations of test compounds in samples were measured by HPLC/MS/MS, samples from the donor compartment were diluted 1:50 (v:v) with assay buffer, samples from receiver compartment were measured without dilution.

Apparent permeabilities in a-b (PappAB) and b-a (PappBA) directions are calculated according to the formula:

$$Papp [ cm / s ] = \frac{1}{A \cdot {C}_{don}} \frac{Vrec \cdot Δ Crec}{Δ t}$$

Vrec [mL]: buffer volume in receiver compartment
Cdon [μmol/mL]: concentration of test compound in donor compartment at t=0
ΔCrec: difference between concentrations of test compound in receiver compartment at start and end of incubation time
Δt: Incubation time
Vrec·ΔCrec/Δt [μmol/min]: Amount of compound transferred to receiver compartment per time
A [cm²]: filter surface

Caco-2 efflux ratios (ER) are calculated as the ratio of PappBA/PappAB.

Table 34 shows data obtained with the disclosed assay for a selection of compounds according to the invention.

TABLE 34

| | | Caco PappAB |
|---|---|---|
| Ex # | Caco ER | (×10⁻⁶ cm/sec) |
| V-1 | 0.7 | 25.6 |
| V-2 | 1.5 | 26.4 |
| V-4 | 9.8 | 5.0 |
| V-5 | 1.7 | 21.4 |
| V-6 | 19.0 | 1.2 |
| V-7 | 0.7 | 32.2 |
| V-8 | 1.3 | 20.9 |
| V-9 | 0.9 | 27.0 |
| V-10 | 3.2 | 11.3 |
| V-11 | 3.0 | 13.0 |
| V-27 | 0.5 | 14.0 |

The formulation examples which follow illustrate the present invention without restricting its scope:

### Examples of Pharmaceutical Formulations

| A) Tablets | per tablet | |
|---|---|---|
| active substance according to the invention | 100 | mg |
| lactose | 140 | mg |
| corn starch | 240 | mg |
| polyvinylpyrrolidone | 15 | mg |
| magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet | |
|---|---|---|
| active substance according to the invention | 80 | mg |
| lactose | 55 | mg |
| corn starch | 190 | mg |
| microcrystalline cellulose | 35 | mg |
| polyvinylpyrrolidone | 15 | mg |
| sodiumcarboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) Tablets | per tablet | |
|---|---|---|
| active substance according to the invention | 25 | mg |
| lactose | 50 | mg |
| microcrystalline cellulose | 24 | mg |
| magnesium stearate | 1 | mg |
| | 100 | mg |

The active substance, lactose and cellulose are mixed together. The mixture is screened, then either moistened with water, kneaded, wet-granulated and dried or dry-granulated or directly final blend with the magnesium stearate and compressed to tablets of suitable shape and size. When wet-granulated, additional lactose or cellulose and magnesium stearate is added and the mixture is compressed to produce tablets of suitable shape and size.

| D) Ampoule solution | | |
|---|---|---|
| active substance according to the invention | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of the formula (V) 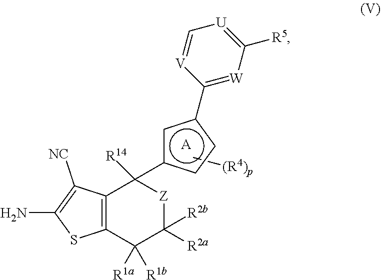
wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached form a cyclopropane ring;
Z is —(CR^{6a}R^{6b})ₙ—;
each R^{6a} and R^{6b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{6a} and R^{6b} together with the carbon atom they are attached to form a cyclopropane ring;
n is selected from the group consisting of 0, 1 and 2;
R¹⁴ is selected from the group consisting of hydrogen, C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
W is nitrogen (—N═) or —CH═;
V is nitrogen (—N═) or —CH═;
U is nitrogen (—N═) or —C(R¹¹)═;
R¹¹ is selected from hydrogen, halogen and C₁₋₄alkoxy;
ring A is a 5 membered heteroarylene;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting of 0, 1, 2 and 3;
R⁵ is halogen or a 3-11 membered heterocyclyl optionally substituted with one or more identical or different C₁₋₆alkyl, C₁₋₆alkoxy, —C(O)—O—C₁₋₆alkyl or a 5-6 membered heterocyclyl, wherein the C₁₋₆alkyl is optionally substituted with cyclopropyl;
or R⁵ is —O—C₁₋₆alkyl substituted with a 3-11 membered heterocyclyl, wherein the 3-11 membered heterocyclyl is optionally substituted with one or more, identical or different R¹², and each R¹² is selected from the group consisting of C₁₋₆alkyl, C₁₋₆alkoxy, —C(O)—O—C₁₋₆alkyl halogen and 3-11 membered heterocyclyl;
or a salt thereof.

2. The compound according to claim 1 of the formula (Va) or a salt thereof

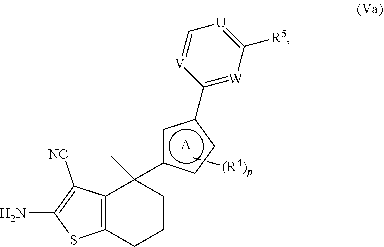

3. The compound according to claim 1 of the formula (Vb) or a salt thereof

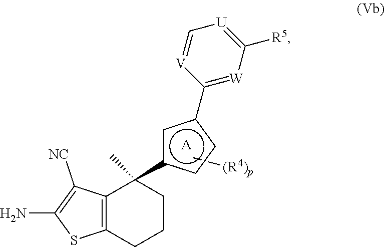

4. The compound or its salt according to claim 1, wherein ring A is selected from

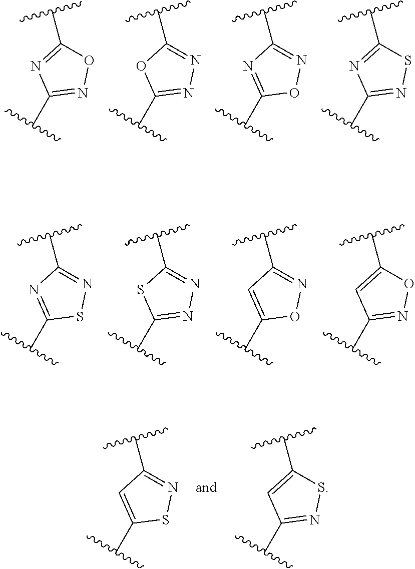

5. The compound according to claim 1 which comprises formula (Vc) or s a salt thereof

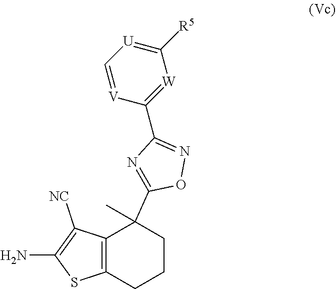

6. The compound according to claim 1 which comprises formula (Vd) or s a salt thereof

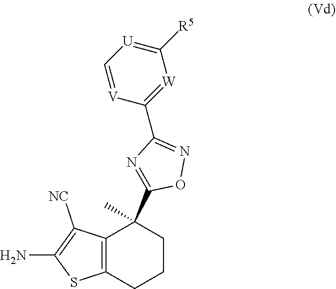

7. The compound or its salt according to claim 1, wherein:
R⁵ is halogen or a 3-11 membered heterocyclyl optionally substituted with one or more identical or different C₁₋₆alkyl, C₁₋₆alkoxy or a 5-6 membered heterocyclyl, wherein the C₁₋₆alkyl is optionally substituted with cyclopropyl;
or R⁵ is —O—C₁₋₆alkyl substituted with a 3-11 membered heterocyclyl, wherein the 3-11 membered heterocyclyl is optionally substituted with one or more, identical or different R¹², each R¹² is selected from the group consisting of C₁₋₆alkyl, C₁₋₆alkoxy, halogen and 3-11 membered heterocyclyl.

8. The compound or its salt according to claim 1, wherein R⁵ is selected from the group consisting of

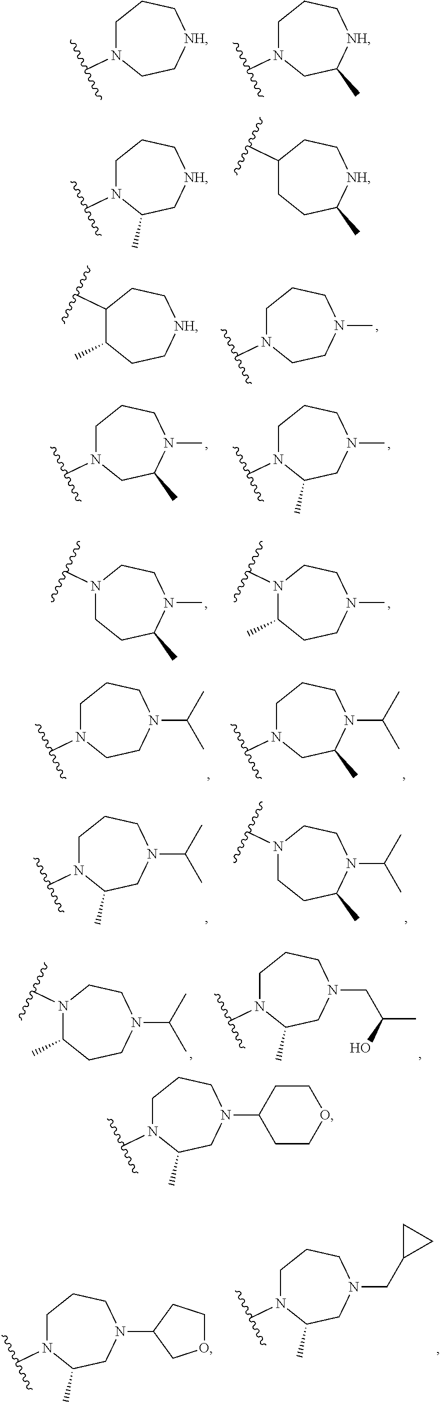

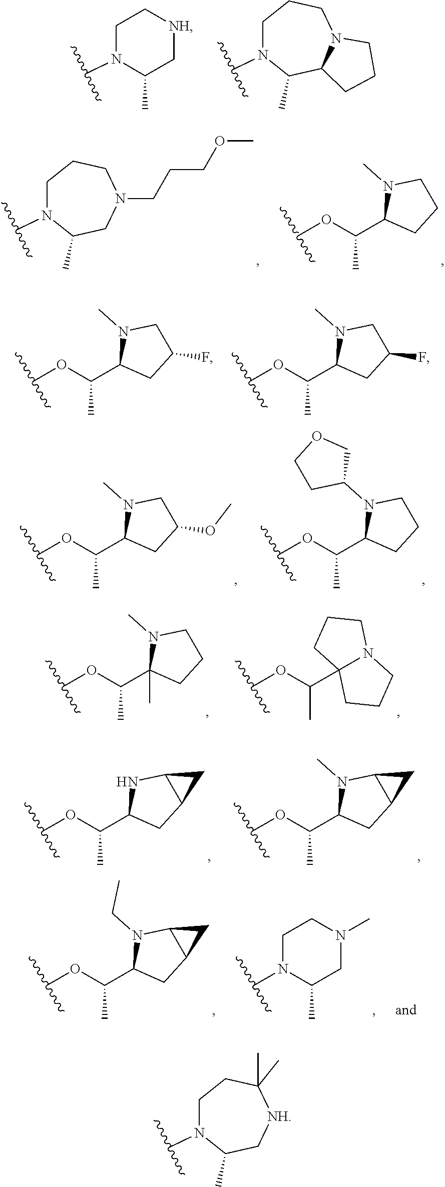

9. The compound or its salt according to claim 1, wherein R⁵ is selected from the group consisting of

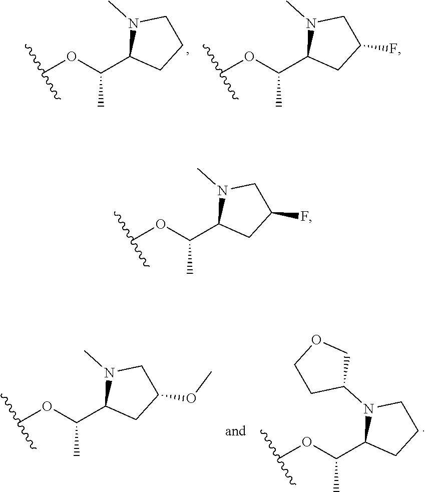

10. The compound or its salt according to claim 1, wherein R⁵ is selected from the group consisting of

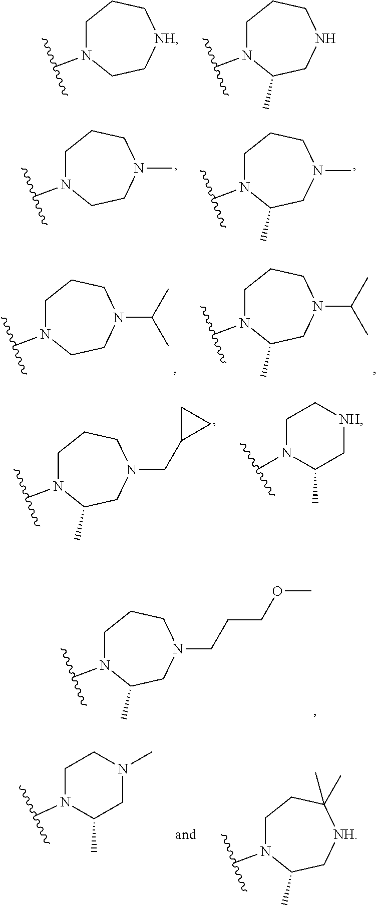

11. The compound or its salt according to claim 1, wherein:
W is nitrogen (—N═);
V is nitrogen (—N═);
U is ═C(R¹¹)—; and
R¹¹ is selected from hydrogen, halogen and C₁₋₄alkoxy.

12. The compound or its salt according to claim 1, wherein:
W is nitrogen (—N═);
V is —CH═; and
U is nitrogen (—N═).

13. The compound according to claim 1, which is selected from the group consisting of: 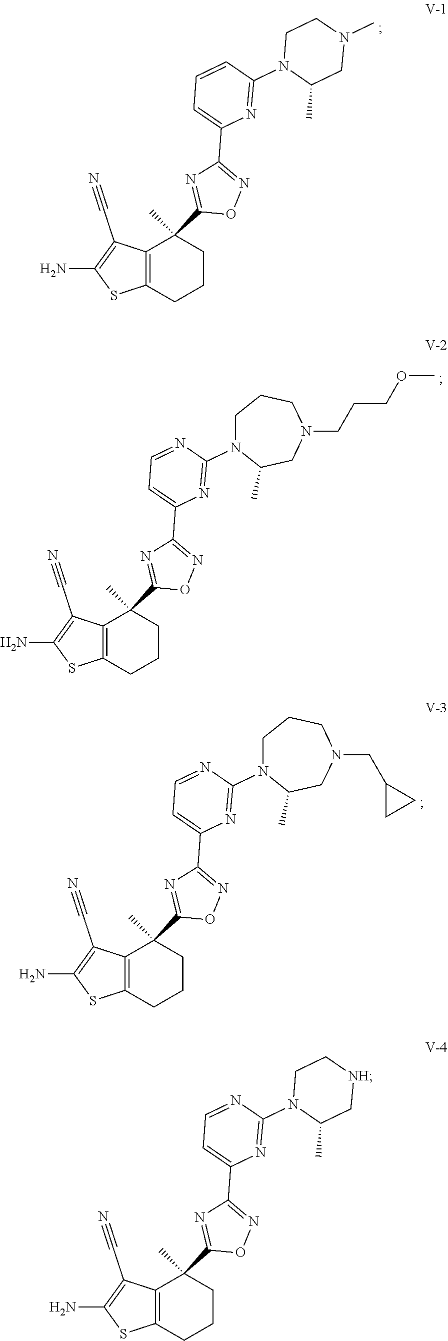 [SMILES: C[C@H]1CN(C)CCN1c1cccc(-c2noc([C@@]3(C)CCCc4sc(N)c(C#N)c43)n2)n1 ; COCCCN1CCCN(c2nccc(-c3noc([C@@]4(C)CCCc5sc(N)c(C#N)c54)n3)n2)[C@@H](C)C1 ; C[C@H]1CN(CC2CC2)CCCN1c1nccc(-c2noc([C@@]3(C)CCCc4sc(N)c(C#N)c43)n2)n1 ; C[C@H]1CNCCN1c1nccc(-c2noc([C@@]3(C)CCCc4sc(N)c(C#N)c43)n2)n1] 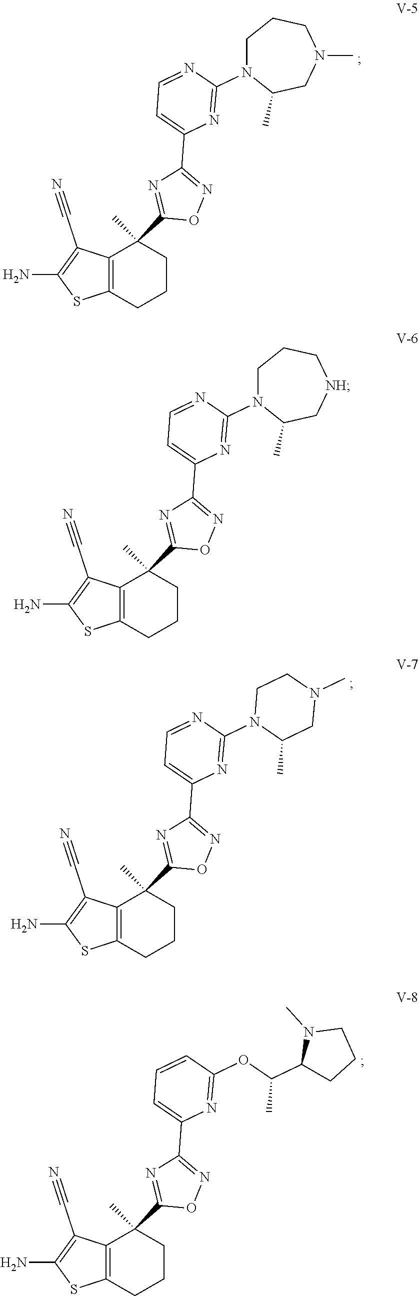 [SMILES: C[C@H]1CN(C)CCCN1c1nccc(-c2noc([C@@]3(C)CCCc4sc(N)c(C#N)c43)n2)n1 ; C[C@H]1CNCCCN1c1nccc(-c2noc([C@@]3(C)CCCc4sc(N)c(C#N)c43)n2)n1 ; C[C@H]1CN(C)CCN1c1nccc(-c2noc([C@@]3(C)CCCc4sc(N)c(C#N)c43)n2)n1 ; C[C@H](Oc1cccc(-c2noc([C@@]3(C)CCCc4sc(N)c(C#N)c43)n2)n1)[C@@H]1CCCN1C] 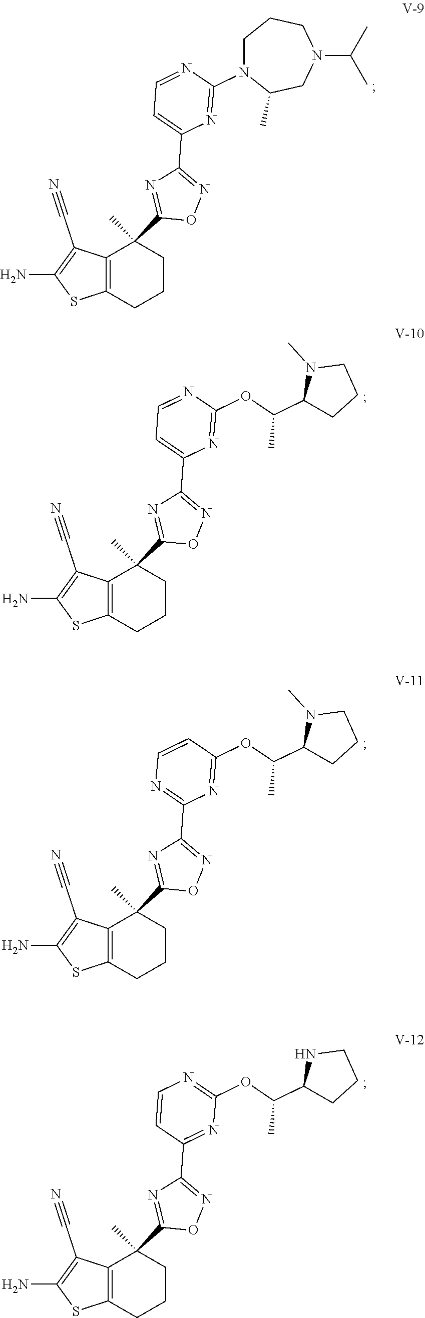 [SMILES: CC(C)N1CCCN(c2nccc(-c3noc([C@@]4(C)CCCc5sc(N)c(C#N)c54)n3)n2)[C@@H](C)C1 ; C[C@H](Oc1nccc(-c2noc([C@@]3(C)CCCc4sc(N)c(C#N)c43)n2)n1)[C@@H]1CCCN1C ; C[C@H](Oc1ccnc(-c2noc([C@@]3(C)CCCc4sc(N)c(C#N)c43)n2)n1)[C@@H]1CCCN1C ; C[C@H](Oc1nccc(-c2noc([C@@]3(C)CCCc4sc(N)c(C#N)c43)n2)n1)[C@@H]1CCCN1] 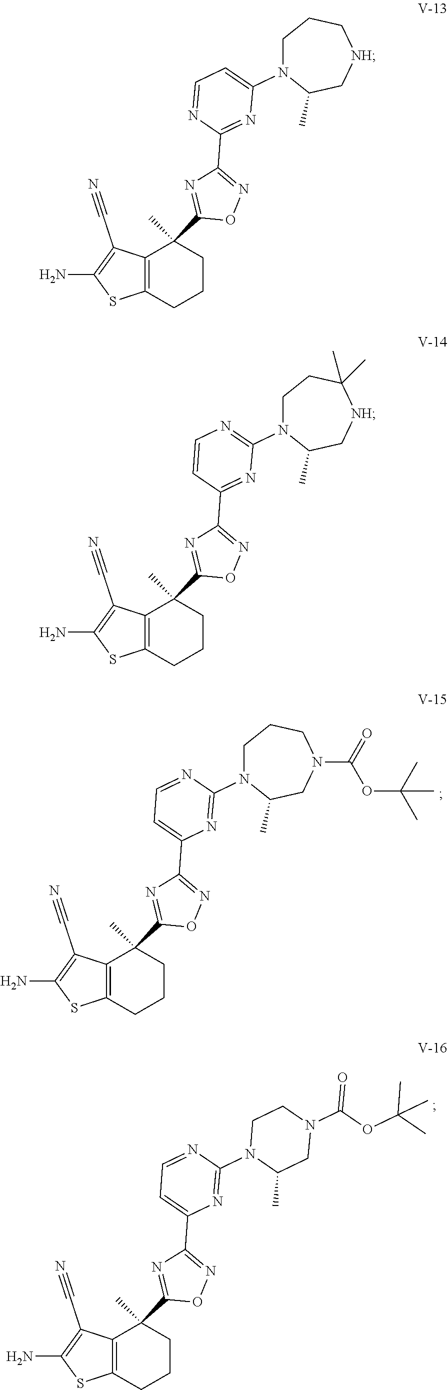 [SMILES: C[C@H]1CNCCCN1c1ccnc(-c2noc([C@@]3(C)CCCc4sc(N)c(C#N)c43)n2)n1 ; C[C@H]1CNC(C)(C)CCN1c1nccc(-c2noc([C@@]3(C)CCCc4sc(N)c(C#N)c43)n2)n1 ; C[C@H]1CN(C(=O)OC(C)(C)C)CCCN1c1nccc(-c2noc([C@@]3(C)CCCc4sc(N)c(C#N)c43)n2)n1 ; C[C@H]1CN(C(=O)OC(C)(C)C)CCN1c1nccc(-c2noc([C@@]3(C)CCCc4sc(N)c(C#N)c43)n2)n1] 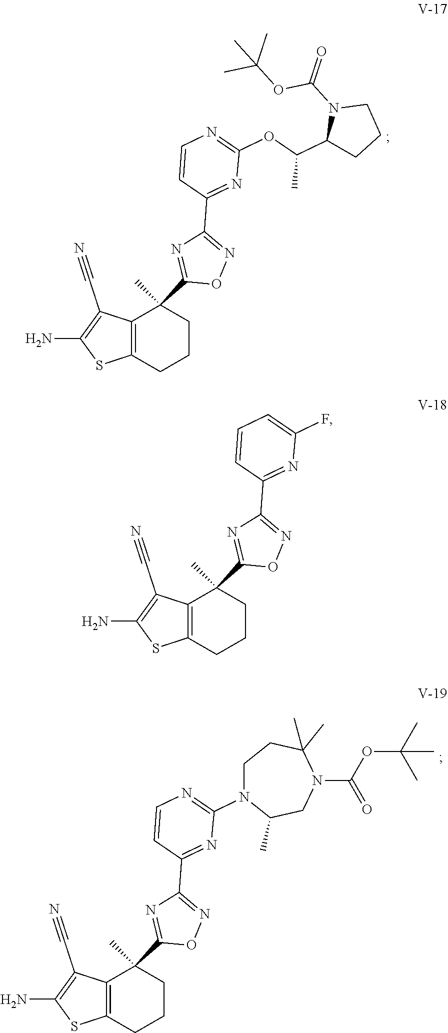 [SMILES: C[C@H](Oc1nccc(-c2noc([C@@]3(C)CCCc4sc(N)c(C#N)c43)n2)n1)[C@@H]1CCCN1C(=O)OC(C)(C)C ; C[C@]1(c2nc(-c3cccc(F)n3)no2)CCCc2sc(N)c(C#N)c21 ; C[C@H]1CN(C(=O)OC(C)(C)C)C(C)(C)CCN1c1nccc(-c2noc([C@@]3(C)CCCc4sc(N)c(C#N)c43)n2)n1] 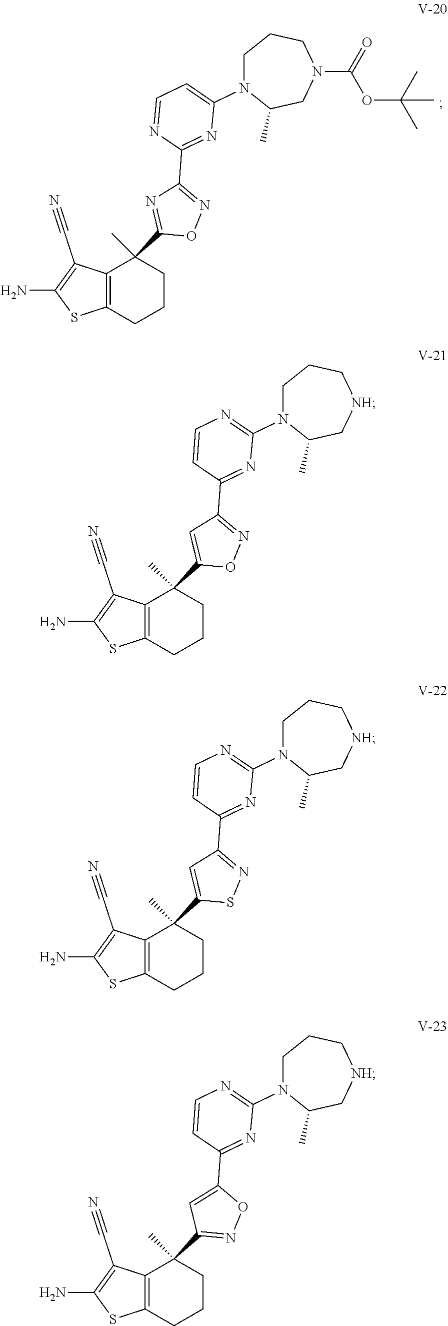 [SMILES: C[C@H]1CN(C(=O)OC(C)(C)C)CCCN1c1ccnc(-c2noc([C@@]3(C)CCCc4sc(N)c(C#N)c43)n2)n1 ; C[C@H]1CNCCCN1c1nccc(-c2cc([C@@]3(C)CCCc4sc(N)c(C#N)c43)on2)n1 ; C[C@H]1CNCCCN1c1nccc(-c2cc([C@@]3(C)CCCc4sc(N)c(C#N)c43)sn2)n1 ; C[C@H]1CNCCCN1c1nccc(-c2cc([C@@]3(C)CCCc4sc(N)c(C#N)c43)no2)n1] 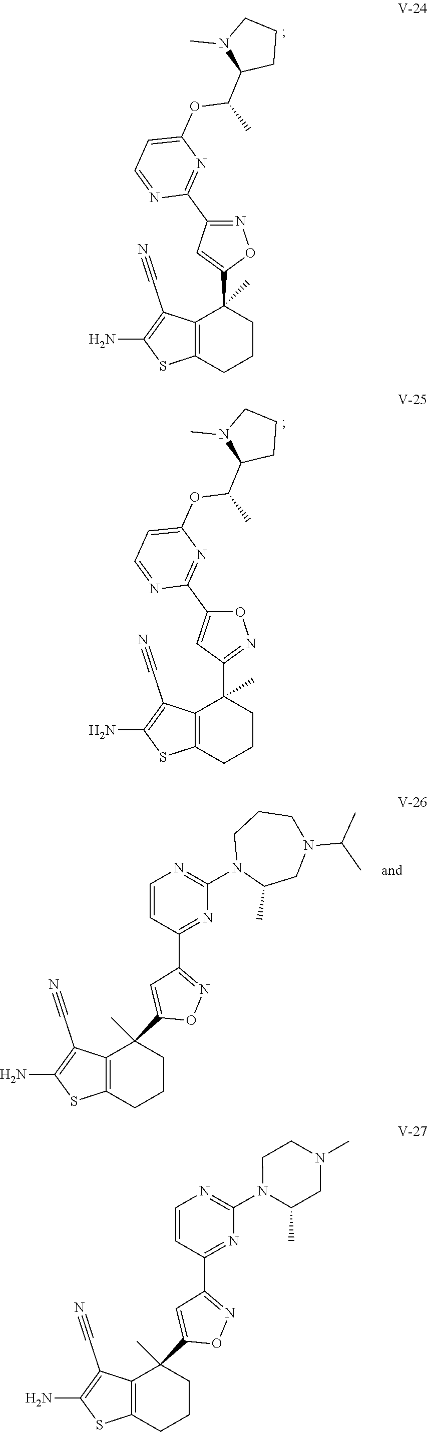
or a pharmaceutically acceptable salt thereof.

14. A method of treatment of a subject in need thereof which comprises administering a therapeutically or prophylactically effective amount of a compound according to claim 1 or a pharmaceutically acceptable salt thereof.

15. A method of treatment and/or prevention of cancer in a subject in need thereof which comprises administering a therapeutically or prophylactically effective amount of a compound according to claim 1 or a pharmaceutically acceptable salt thereof.

16. The method of claim 15, wherein said compound or salt is administered in combination with one or more other pharmacologically active substance(s).

17. The method of claim 15, wherein the cancer is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, gastroesophageal cancer, chronic lymphocytic leukemia, hepatocellular cancer, breast cancer, ovarian cancer, prostate cancer, glioblastoma, renal cancer and sarcoma.

18. The method of claim 15, wherein the cancer comprises tumor cells comprising a KRAS mutation or an amplification of KRAS wildtype.

19. The method of claim 18, wherein the KRAS mutation is selected from the group consisting of: KRAS G12C, KRAS G12D, KRAS G12V and KRAS G13D.

20. A pharmaceutical composition comprising a compound according to claim 1 or a pharmaceutically acceptable salt thereof and one or more other pharmaceutically acceptable excipient(s).

21. A compound according to claim 13 with the structure V-11, or a pharmaceutically acceptable salt thereof: 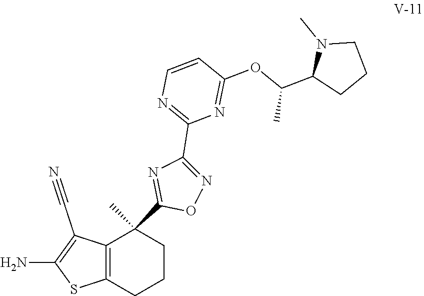 [SMILES: C[C@H](Oc1ccnc(-c2noc([C@@]3(C)CCCc4sc(N)c(C#N)c43)n2)n1)[C@@H]1CCCN1C]
